SEQUENCE LISTING (62 sequences)

SEQ ID NO: 1 - DNA, 15 bp, synthetic construct
  source 1..15: mol_type = other DNA
  misc_feature <1: note = 5IRD800CWN
ggttccaatcggatc

SEQ ID NO: 2 - DNA, 15 bp, synthetic construct
  source 1..15: mol_type = other DNA
  misc_feature <1: note = 5IRD800CWN
cgaacgaacgatagc

SEQ ID NO: 3 - DNA, 15 bp, synthetic construct
  source 1..15: mol_type = other DNA
  misc_feature <1: note = 5IRD800CWN
tcggacgatcatggg

SEQ ID NO: 4 - DNA, 15 bp, synthetic construct
  source 1..15: mol_type = other DNA
  misc_feature <1: note = 5IRD800CWN
attgaccgtctcgtt

SEQ ID NO: 5 - DNA, 15 bp, synthetic construct
  source 1..15: mol_type = other DNA
  misc_feature <1: note = 5IRD800CWN
attagggcatcgacc

SEQ ID NO: 6 - DNA, 15 bp, synthetic construct
  source 1..15: mol_type = other DNA
  misc_feature >15: note = 3Cy5Sp
gcgcagcaattcact

SEQ ID NO: 7 - DNA, 15 bp, synthetic construct
  source 1..15: mol_type = other DNA
  misc_feature >15: note = 3Cy5Sp
ggtcccgttgaactt

SEQ ID NO: 8 - DNA, 15 bp, synthetic construct
  source 1..15: mol_type = other DNA
  misc_feature <1: note = 5IRD800CWN
agcgcgtcaaacaga

SEQ ID NO: 9 - DNA, 15 bp, synthetic construct
  source 1..15: mol_type = other DNA
  misc_feature <1: note = 5Alex594N
aacgagcgtcccttg

SEQ ID NO: 10 - DNA, 15 bp, synthetic construct
  source 1..15: mol_type = other DNA
  misc_feature <1: note = 5Alex594N
cgttgcgacgactaa

SEQ ID NO: 11 - DNA, 15 bp, synthetic construct
  source 1..15: mol_type = other DNA
  misc_feature >15: note = 3Cy5Sp
caccgttgcgcttac

SEQ ID NO: 12 - DNA, 15 bp, synthetic construct
  source 1..15: mol_type = other DNA
  misc_feature <1: note = 5Alex594N
tccgtcacgcaattt

SEQ ID NO: 13 - DNA, 15 bp, synthetic construct
  source 1..15: mol_type = other DNA
  misc_feature <1: note = 5Alex594N
cgtagcggaatctgc

SEQ ID NO: 14 - DNA, 15 bp, synthetic construct
  source 1..15: mol_type = other DNA
  misc_feature <1: note = 5Alex594N
gtcgggaacggatac

SEQ ID NO: 15 - DNA, 15 bp, synthetic construct
  source 1..15: mol_type = other DNA
  misc_feature <1: note = 5Alex594N
gatgtaattcggccg

SEQ ID NO: 16 - DNA, 15 bp, synthetic construct
  source 1..15: mol_type = other DNA
  misc_feature <1: note = 5IRD800CWN
tatgtaagtgggtgg

SEQ ID NO: 17 - DNA, 15 bp, synthetic construct
  source 1..15: mol_type = other DNA
  misc_feature >15: note = 3Cy5Sp
ttagggaggtgggtg

SEQ ID NO: 18 - DNA, 15 bp, synthetic construct
  source 1..15: mol_type = other DNA
  misc_feature <1: note = 5Alex594N
gttaggatgggttgt

SEQ ID NO: 19 - DNA, 15 bp, synthetic construct
  source 1..15: mol_type = other DNA
  misc_feature <1: note = 5IRD800CWN
gaagggagtaattga

SEQ ID NO: 20 - DNA, 15 bp, synthetic construct
  source 1..15: mol_type = other DNA
  misc_feature >15: note = 3Cy5Sp
ggagatgttgtgaag

SEQ ID NO: 21 - DNA, 15 bp, synthetic construct
  source 1..15: mol_type = other DNA
  misc_feature >15: note = 3Cy5Sp
gtgatgtagtgggat

SEQ ID NO: 22 - DNA, 15 bp, synthetic construct
  source 1..15: mol_type = other DNA
  misc_feature >15: note = 3Cy5Sp
gaaggagtagaggag

SEQ ID NO: 23 - DNA, 15 bp, synthetic construct
  source 1..15: mol_type = other DNA
  misc_feature >15: note = 3Cy5Sp
cctaaggcaacgagt

SEQ ID NO: 24 - DNA, 15 bp, synthetic construct
  source 1..15: mol_type = other DNA
  misc_feature >15: note = 3Cy5Sp
atggacctgctcagt

SEQ ID NO: 25 - DNA, 15 bp, synthetic construct
  source 1..15: mol_type = other DNA
  misc_feature >15: note = 3Cy5Sp
tcatccctgtgccat

SEQ ID NO: 26 - DNA, 15 bp, synthetic construct
  source 1..15: mol_type = other DNA
  misc_feature >15: note = 3Cy5Sp
aatgacgcagactcg

SEQ ID NO: 27 - DNA, 15 bp, synthetic construct
  source 1..15: mol_type = other DNA
  misc_feature >15: note = 3Cy5Sp
caatagtccagttcg

SEQ ID NO: 28 - DNA, 15 bp, synthetic construct
  source 1..15: mol_type = other DNA
  misc_feature >15: note = 3Cy5Sp
ctaaggttccctcag

SEQ ID NO: 29 - DNA, 15 bp, synthetic construct
  source 1..15: mol_type = other DNA
  misc_feature >15: note = 3Cy5Sp
gatgcctccgtatct

SEQ ID NO: 30 - DNA, 15 bp, synthetic construct
  source 1..15: mol_type = other DNA
  misc_feature >15: note = 3Cy5Sp
gcagaatggtaaggg

SEQ ID NO: 31 - DNA, 15 bp, synthetic construct
  source 1..15: mol_type = other DNA
  misc_feature <1: note = 5IRD800CWN
tatgctcactcgctg

SEQ ID NO: 32 - DNA, 15 bp, synthetic construct
  source 1..15: mol_type = other DNA
  misc_feature <1: note = 5IRD800CWN
ctgcgatacattgtg

SEQ ID NO: 33 - DNA, 15 bp, synthetic construct
  source 1..15: mol_type = other DNA
  misc_feature <1: note = 5IRD800CWN
cctactgacaccgta

SEQ ID NO: 34 - DNA, 15 bp, synthetic construct
  source 1..15: mol_type = other DNA
  misc_feature <1: note = 5IRD800CWN
gacaaccgtaaagag

SEQ ID NO: 35 - DNA, 15 bp, synthetic construct
  source 1..15: mol_type = other DNA
  misc_feature <1: note = 5IRD800CWN
acagtagtgccgttg

SEQ ID NO: 36 - DNA, 15 bp, synthetic construct
  source 1..15: mol_type = other DNA
  misc_feature <1: note = 5IRD800CWN
ggagcccgtaagtat

SEQ ID NO: 37 - DNA, 15 bp, synthetic construct
  source 1..15: mol_type = other DNA
  misc_feature <1: note = 5IRD800CWN
accatcaatgctcgt

SEQ ID NO: 38 - DNA, 15 bp, synthetic construct
  source 1..15: mol_type = other DNA
  misc_feature <1: note = 5IRD800CWN
cacccttgggcttat

SEQ ID NO: 39 - DNA, 15 bp, synthetic construct
  source 1..15: mol_type = other DNA
  misc_feature <1: note = 5IRD800CWN
ccatttggcgtgaag

SEQ ID NO: 40 - DNA, 15 bp, synthetic construct
  source 1..15: mol_type = other DNA
  misc_feature <1: note = 5IRD800CWN
ggaagagtgctcata

SEQ ID NO: 41 - DNA, 15 bp, synthetic construct
  source 1..15: mol_type = other DNA
  misc_feature >15: note = 3AlexF594N
gaatgcgatgtgtcc

SEQ ID NO: 42 - DNA, 15 bp, synthetic construct
  source 1..15: mol_type = other DNA
  misc_feature >15: note = 3AlexF594N
gcctatgacaaggat

SEQ ID NO: 43 - DNA, 15 bp, synthetic construct
  source 1..15: mol_type = other DNA
  misc_feature >15: note = 3AlexF594N
tagcgagaatcgtgg

SEQ ID NO: 44 - DNA, 15 bp, synthetic construct
  source 1..15: mol_type = other DNA
  misc_feature >15: note = 3AlexF594N
ctcgcaatgtgacaa

SEQ ID NO: 45 - DNA, 15 bp, synthetic construct
  source 1..15: mol_type = other DNA
  misc_feature >15: note = 3AlexF594N
ttgaggtgcgaagtc

SEQ ID NO: 46 - DNA, 15 bp, synthetic construct
  source 1..15: mol_type = other DNA
  misc_feature >15: note = 3AlexF594N
ttctgtcctcggtga

SEQ ID NO: 47 - DNA, 15 bp, synthetic construct
  source 1..15: mol_type = other DNA
  misc_feature >15: note = 3AlexF594N
cgttcacggctgata

SEQ ID NO: 48 - DNA, 15 bp, synthetic construct
  source 1..15: mol_type = other DNA
  misc_feature >15: note = 3AlexF594N
cactacgcttgtgac

SEQ ID NO: 49 - DNA, 15 bp, synthetic construct
  source 1..15: mol_type = other DNA
  misc_feature >15: note = 3AlexF594N
aaatgtgtgggcgaa

SEQ ID NO: 50 - DNA, 15 bp, synthetic construct
  source 1..15: mol_type = other DNA
  misc_feature >15: note = 3AlexF594N
gtcctctgctacagt

SEQ ID NO: 51 - DNA, 15 bp, synthetic construct
  source 1..15: mol_type = other DNA
  misc_feature >15: note = 3Cy5Sp
aggagcagtagacag

SEQ ID NO: 52 - DNA, 15 bp, synthetic construct
  source 1..15: mol_type = other DNA
  misc_feature <1: note = 5IRD800CWN
gtaaccgagtggcat

SEQ ID NO: 53 - DNA, 20 bp, synthetic construct
  source 1..20: mol_type = other DNA
ctatgcgctatcccggacgc

SEQ ID NO: 54 - DNA, 38 bp, synthetic construct
  source 1..38: mol_type = other DNA
taatacgactcactatagggtcgcatatccgtaccggc

SEQ ID NO: 55 - DNA, 21 bp, synthetic construct
  source 1..21: mol_type = other DNA
ccgttcaagactgccgtgcta

SEQ ID NO: 56 - DNA, 40 bp, synthetic construct
  source 1..40: mol_type = other DNA
taatacgactcactatagggctagggagcctacaggctgc

SEQ ID NO: 57 - DNA, 20 bp, synthetic construct
  source 1..20: mol_type = other DNA
ttgcgttcggtctgaatgcg

SEQ ID NO: 58 - DNA, 40 bp, synthetic construct
  source 1..40: mol_type = other DNA
taatacgactcactatagggactcctgctctttgggtccg

SEQ ID NO: 59 - DNA, 20 bp, synthetic construct
  source 1..20: mol_type = other DNA
cgccctaatctccgcttggg

SEQ ID NO: 60 - DNA, 40 bp, synthetic construct
  source 1..40: mol_type = other DNA
taatacgactcactataggggcttcgaccgagggcgaaat

SEQ ID NO: 61 - DNA, 20 bp, synthetic construct
  source 1..20: mol_type = other DNA
tgcccgcctttcgttactca

SEQ ID NO: 62 - DNA, 40 bp, synthetic construct
  source 1..40: mol_type = other DNA
taatacgactcactatagggcgcaatcgtcggctaacggt

(19) United States
(12) Patent Application Publication
(10) Pub. No.: US 2026/0201454 A1
(43) Pub. Date: Jul. 16, 2026

(54) Method of In Situ Cell Characterization

(71) Applicant: AGENCY FOR SCIENCE, TECHNOLOGY AND RESEARCH, Singapore (SG)

(72) Inventors: Kok Hao Chen, Singapore (SG); Xinrui Zhou, Singapore (SG); Wan Yi Seow, Singapore (SG); How Ong Norbert Ha, Singapore (SG); Jeeranan Boonruangkan, Singapore (SG); Shijie Nigel Chou, Singapore (SG); Jie Lin Jolene Goh, Singapore (SG)

(21) Appl. No.: 19/133,863

(22) PCT Filed: Nov. 29, 2023
(86) PCT No.: PCT/SG2023/050790

§ 371 Date: May 29, 2025

(30) Foreign Application Priority Data
Nov. 29, 2022 (SG) 10202260245V

(51) Int. Cl. C12Q 1/6841; C12Q 1/6886

(52) U.S. Cl. CPC C12Q 1/6841; C12Q 1/6886; C12Q 2600/118; C12Q 2600/158

(57) ABSTRACT

This technology relates to a method and kit for characterizing cells in a biological sample in situ.

### CROSS-REFERENCES

This application claims priority to Singapore patent application 10202260245V, filed on 29 Nov. 2022, which is expressly incorporated herein by reference in its entirety, with particular reference to the figures, legends, and claims therein.

### FIELD OF THE INVENTION

The present invention relates generally to the field of molecular and cell biology. In particular, the present invention relates to methods of cell characterisation.

### BACKGROUND

High-dimensional, spatially resolved analysis of intact biological tissue samples promises to transform biomedical research and diagnostics. Recent advancements in single-cell RNA-sequencing (scRNA-seq) make it possible to unbiasedly define cell types reflecting ontogeny, functions, or anatomical locations. However, high-throughput mapping of these cells within intact biological systems is still a technical challenge. Existing methods such as spatial indexing combined with next-generation sequencing has enabled spatial mapping of sequencing reads and in situ reconstructions of cell types. However, sequencing-based spatial transcriptomics methods are limited by RNA diffusion and capture efficiency. Alternatively, cell types can also be characterised via imaging-based spatial transcriptomics methods, by targeting RNAs with multiplexed single-molecule Fluorescence In situ Hybridisation (FISH) or in situ sequencing. Such methods are highly quantitative and scalable to the whole transcriptome (~10,000 genes), but suffer from disadvantages including high non-specific background noises, limitation by molecular crowding, and the requirement of high-resolution microscopes. The imaging-based spatial transcriptomics methods also become increasingly laborious with larger number of targets. Another approach for spatial mapping of cells is multiplexed immunostaining or spatial proteomics. While the increased copy number of proteins compared to RNAs may lead to an increase in detection robustness, antibody panels are more costly, less flexible, with poor scalability.

Therefore, what is needed is a technology that enables easy, efficient and a scalable method for spatial characterisation of cells within the context of normal tissue physiology or disease microenvironment. Furthermore, other desirable features and characteristics will become apparent from the subsequent detailed description and the appended claims, taken in conjunction with the accompanying drawings referred to herein.

### SUMMARY OF INVENTION

In one aspect, the present disclosure refers to a method of characterizing cells in a biological sample in situ, comprising: a. contacting the biological sample with a plurality of probes that bind to ribonucleic acid (RNA) transcripts of a plurality of pre-determined genes, wherein each probe comprises i) a detectable label, and ii) a domain that binds specifically to a ribonucleic acid transcript of one of the pre-determined genes; wherein a signal is emitted when the probe binds to the ribonucleic acid transcript; b. detecting a combination or plurality of emitted signals from the plurality of probes; and c. characterizing the cells based on the combination or plurality of emitted signals.

In another aspect, the present disclosure refers to a method to determine the prognosis of a subject suffering from cancer, comprising: a. obtaining a sample of the subject; b. characterizing one or more cancer cells in the sample using the method of any one of claims 1 to 13 to determine the stage of the cancer; and c. determining the prognosis based on the stage of the cancer.

In another aspect, the present disclosure refers to a kit for characterising cells in a biological sample in situ comprising: a plurality of probes that bind to ribonucleic acid (RNA) transcripts of a plurality of pre-determined genes; wherein each probe comprises i) a detectable label, and ii) a domain that binds specifically to a ribonucleic acid transcript of one of the pre-determined genes, and instructions for use.

In another aspect, the present disclosure refers to a kit for characterizing a colorectal cancer in a biological sample in situ comprising: a plurality of probes that bind to ribonucleic acid (RNA) transcripts of a plurality of pre-determined genes, wherein the plurality of pre-determined genes is selected from the genes listed in Table 6 (6a)-(6d); wherein each probe comprises: i) a detectable label, and ii) a domain that binds specifically to a ribonucleic acid transcript of the plurality of pre-determined genes, and instructions for use.

### BRIEF DESCRIPTION OF THE DRAWINGS

FIG. 1 provides a schematic overview of the in situ hybridisation (ISH) method as described herein for characterisation of cells. The method as described herein can be used for accurate mapping of cell types without disrupting the tissue architecture. As described herein, the method is a sensitive, robust, and scalable in situ hybridisation (ISH)-based spatial transcriptomics method that profiles single cells using multiple co-regulated genes. As used herein, co-regulated genes refers to genes that show coordinated changes in the gene expression level, i.e. covarying genes. As shown in FIG. 1A, co-regulated genes are spatially co-localized in the same cells within a tissue, which allows designing of hybridisation probes to target a large set of genes for reliable detection of a cell population of interest. FIG. 1A provides a cell-by-gene count matrix from single-cell RNA sequencing (scRNA-seq). The matrix is used to cluster cell types, which are characterized by their unique gene expression profiles (for example, genes A-D are grouped for one cluster of cells and genes E-I are grouped as a different cluster). FIG. 1B provides a graphical illustration of the identification of groups of correlated genes from the reference scRNA-seq data. Genes that show coordinated changes in expression levels with each other are spatially co-localized in the same cells within a tissue. Based on the groups of correlated genes identified, thousands of oligonucleotide probes against their transcripts were designed, which resulted in tens of thousands of detectable tags per cell (factoring in number of genes, transcript copy number per cell, and number of probes per transcript). By designing labelled oligonucleotide probes that target a large set of co-regulated transcripts, the in situ hybridisation cell characterisation method as described herein improves the intensity of signal detection. FIG. 1C demonstrates the workflow of the in situ hybridisation-based expression profiling of cells in combination with the array-synthesized oligo-pool and sequential fluidics technologies in animal tissues, such as kidney and brain. The method could be applied to healthy tissue or diseased tissues, for example, a normal tissue or a cancer tissue. Combined with repeated rounds of hybridisation and washing, the in situ hybridisation method for characterisation of cells as described herein enables robust and scalable mapping of cell types in tissue samples. Commonly used detectable signals are, for example, fluorescent signals. One useful application of the in situ hybridisation method can be fluorescence in situ hybridisation for characterisation of cellular heterogeneity (referred to as “FISHnCHIPS” in some specific examples). Therefore, the present disclosure provides, as summarised herein, a robust in situ hybridisation method for characterising cells in a biological sample, with amplified signal intensity and high scalability.

FIG. 2 provides a comparison of an exemplary application of the present method and a conventional single-molecule RNA FISH (smFISH) in an exemplary mouse kidney tissue. In the exemplary method shown in this Figure (“FISHnCHIPS”), fluorescently labelled probes were designed using a mouse kidney scRNA-seq dataset for five selected cell types: renal macrophages, glomerular endothelial cells, loop of Henle (LOH) cells, collecting duct (CD) cells, and glomerular podocytes. FIG. 2A provides a gene expression heatmap generated based on the scRNA-seq reference data highlighting the five corresponding cell clusters representative for each cell type. A suitable cut-off value is applied to the correlation coefficient calculated for the genes to determine the genes to be targeted using FISHnCHIP for each cell type. The heatmap shows the relative expression levels of 84 genes that are correlated to the top differentially expressed (DE) genes in the five selected cell types, sampling a maximum of 300 cells per cluster. FIG. 2B shows the unprocessed smFISH images of a mouse kidney tissue slice in the five selected cell types in the left and middle panels, with FISHnCHIPS images in the right panels which labels multiple co-regulated genes simultaneously (14 to 23 genes, as shown in FIG. 2B) to detect target cell types. The smFISH and FISHnCHIPs images are scaled to the same camera intensity range for each cell type. Nuclei staining is shown with DAPI. Scale bar is 3 μm. From the comparison between smFISH and FISHnCHIPs images in FIG. 2B, a high degree of co-localisation between the top two co-regulated genes in each of these cell types are observed, confirming that correlated genes from scRNA-seq are indeed spatially co-localized in the same cells. FIG. 2C shows a FISHnCHIPs image of five different cell types of a mouse kidney tissue. Panel (i) shows a FISHnCHIPs image of endothelial cells of a mouse kidney tissue. Panel (ii) shows a FISHnCHIPs image of collecting duct cells of a mouse kidney tissue. Panel (iii) shows a FISHnCHIPs image of podocyte cells of a mouse kidney tissue. Panel (iv) shows a FISHnCHIPs image of loop of Henle cells of a mouse kidney tissue. Panel (v) shows a FISHnCHIPs image of macrophage cells of a mouse kidney tissue. Panel (vi) shows a DAPI image of the cell nuclei in the same mouse kidney tissue. Scale bar is 25 μm for all images in FIG. 2D. As demonstrated in FIGS. 2, when using a combination of a plurality of genes to label selected cell types, the cells were much more easily detected compared to labelling only a single top differentially expressed (DE) gene. Although these 5 cell types represent only ~12% of the total kidney cell population (estimated from scRNA-seq), the method shown in this Figure reveals intricate spatial details of the kidney tissue architecture, such as the arrangement of podocytes in the highly fenestrated Bowman's capsule, where they wrap around the glomerular endothelial cells. FIG. 2 therefore, provides an example of the cell-centric strategy of the in situ hybridisation (ISH) method for characterisation of cells described herein, which amplifies the detectable signal based on multiple co-regulated genes corresponding to known cell-types that are pre-defined by the user (for example, renal macrophages, glomerular endothelial cells, loop of Henle (LOH) cells, collecting duct (CD) cells, and glomerular podocytes).

FIG. 3 provides a quantification of the exemplary cell-centric FISHnCHIPs signal reading in for the five cell types in mouse kidney in connection with FIG. 2. FIG. 3A shows a boxplot of the ratio of mean fluorescence intensity per cell of FISHnCHIPs to single-molecule FISH (smFISH) (solid box), which indicates the actual increase in fluorescence intensity measured; and the ratio of counts for 14-23 genes to the top DE gene (open box) based on scRNA-seq results, which indicates the predicted value for fluorescence intensity increase. The number of cells calculated for FISHnCHIPs is: collecting duct: 146, podocytes: 461, loop of Henle: 727, endothelial: 400, and macrophage: 341. The number of cells calculated for scRNA-seq is: collecting duct: 1,825, podocytes: 77, loop of Henle: 1,496, endothelial: 701, and macrophage: 216. The box plot shows the median (centre line), the first and third quartiles (box limits), and 1.5× the interquartile range (whiskers). Horizontal line indicates where the fluorescence signal gain is 1. The FISHnCHIPs fluorescence intensity per cell was increased by about 6 to 39-fold across the 5 cell types (median of at least 146 cells) compared to conventional method single-molecule FISH (smFISH), and is consistent with or beyond the predicted signal increase. However, in accordance with the scRNA-seq data as shown in FIG. 2A, some of the selected genes for FISHnCHIPs may be expressed in off-target cell types. For example, Slc5a3, which has a Pearson's correlation (r) of 0.33 to Slc12a1 (a marker for loop of Henle (LOH)), is also expressed in collecting duct (CD) cells. To estimate the crosstalk in the FISHnCHIPs results, the Manders' overlap coefficient is calculated across the five cell-type channels, which ranged from 0.001 to 0.09, suggesting minimal crosstalk for these cell types. FIG. 3B provides a heatmap showing the normalized mean scRNA-seq counts for the selected genes for FISHnCHIPs across the 5 cell types, which is the predictive signal crosstalk level. FIG. 3C shows the Mander's overlap coefficient across the 5 cell-type channels measured by FISHnCHIPS, indicating the actual measured signal crosstalk in the FISHnCHIPs imaged results. The numbers of cells analysed are the same in both FIG. 3B and FIG. 3C. Thus, based on the quantified comparison between a conventional smFISH method and the FISHnCHIPs method as exemplified herein, the present method shows up to 39 folds increase in signal intensity. Further comparison with predictive crosstalk based on scRNA-seq data shows the FISHnCHIPs method as exemplified herein displays minimal crosstalk between cell-types, therefore showing high specificity.

FIG. 4 provides a computational prediction of signal gain and specificity for the cell-centric FISHnCHIPs method as demonstrated in FIG. 2. As shown in FIG. 4A, the heatmap provides visualisation of scRNA-seq gene expression of a FISHnCHIPs gene panel targeting all the previously annotated mouse kidney cell types, sampling a maximum of 300 cells per cluster. FIG. 4B provides the predicted Signal Gain (SG) and Signal Specificity Ratio (SSR) based on the scRNA-seq reference data, both expressed as a function of the number of genes used (ranked by their Pearson's correlation to the top Differentially Expressed gene). The Signal Gain (SG) is defined as the ratio of the sum of counts for FISHnCHIPs genes to that of the top DE gene, and the Signal Specificity Ratio (SSR) is defined as the ratio of the sum of counts for FISHnCHIPs genes in the target cell type to that in the most likely off-target cell type. When SSR approaches unity, the fluorescence intensity for the cell type of interest should be equal to that of an off-target cell type, rendering them indistinguishable. The high Signal Gain (SG) indicates the expected signal amplification for FISHnCHIPs. As shown in FIG. 4B, 9 out of the 16 previously annotated cell types have a SSR of more than 4, which show high specificity for these cell types when using the cell-centric strategy for FISHnCHIPS panel design. FIG. 4C provides an overview of the predicted signal crosstalk in a heatmap showing the normalized mean scRNA-seq counts of the FISHnCHIPs gene panel across all kidney cell types. Despite the enhancement in signal-to-noise ratio, specificity for these cell types using the cell-centric based FISHnCHIPs could be further improved. In view of the predicted signal gain and specificity for the method as described (cell-centric strategy), it is shown that the method results in improved sensitivity, which comes with minimal trade-off in specificity.

FIG. 5 provides an alternative example of the in situ hybridisation (ISH) cell characterisation method as described herein. Instead of cell-centric strategy, which requires user input of known cell type information, the gene-centric strategy utilises correlated genes from clusters of gene expression programs (i.e. coregulated genes within a biological pathway). FIG. 5 shows an exemplary gene-centric FISHnCHIPs profiling of 18 gene modules in mouse cortex. To reduce crosstalk, the genes are clustered based on pathways and gene expression programs, which are known to exhibit coordinated expression variability in at least mammalian genomes, without a priori clustering of cell types. The clustering of the gene-gene correlation matrix (instead of the gene-cell matrix) of a mouse visual cortex dataset is performed. A total of 255 candidate genes are selected, which are highly correlated (Pearson's correlation (r)>0.7) to at least three genes. From the candidate pool, 18 gene modules with significant enrichment for Gene Ontology (GO) are identified. FIG. 5A provides a gene-gene correlation heatmap (of the pairwise Pearson's correlation (r) coefficients) grouped into 18 clusters of gene modules (gene expression programs) based on the identification. Each module (comprises 14 genes on average) is imaged sequentially in a fresh frozen mouse brain tissue section under an automated fluidics-coupled fluorescence microscope system. Exemplary FISHnCHIPs images of a mouse brain tissue slice are stained for gene module 1, 2, 3, and 18. Scale bar is 50 μm for all images. Single cells in the images are segmented using DAPI stain and the cell masks were applied to define 6,180 cells after quality control. The mean fluorescence intensity per cell for each imaged module is quantified. FIG. 5B provides a heat map showing the mean fluorescence intensity per cell. The cell-by-module intensity matrix was clustered using the Louvain algorithm, resulting in eight cell clusters. The cell clusters generated are then targeted respectively in the sample and the detectable labels are measured. FIG. 5C shows spatial maps of the detected cells in panels (i) to (viii), which are separated by cell types into: Glutamatergic neurons (i), GABAergic neurons (ii), Astrocytes (iii), Oligodendrocytes (iv), Endothelial cells (v), Microglial cells (vi), Peri-vascular cells (vii), and Vascular leptomeningeal cells (viii). Scale bars in FIG. 5C are 500 μm. The eight cell types exhibit differential spatial organization patterns as demonstrated in FIG. 5C. To verify whether the identified cell types are consistent with existing methods, FIG. 5D shows the frequency of cell types detected by FISHnCHIPs versus the frequency of cell types detected by Multiplexed Error-Robust Fluorescence In situ Hybridisation (MERFISH) method (Pearson's correlation r=0.97) in a scatter plot. The insert is a pie chart showing the proportion of each FISHnCHIPs cluster. FISHnCHIPs demonstrates high correlation and consistency with existing state of the art method. Therefore, FIG. 5 provides an example of the gene-centric in situ hybridisation (ISH) cell characterisation method, which effectively profiles a tissue sample into eight different cell types based on 18 gene expression programs, showing consistent results with existing method.

FIG. 6 provides further detail on the panel design of the 18 gene expression programs and the resulting clustering of 8 cell types using gene-centric FISHnCHIPs in mouse cortex as shown in FIG. 5. FIG. 6A provides a Uniform Manifold Approximation and Projection (UMAP) representation of the predicted clusters from scRNA-seq simulated module-cell (meta-gene) expression, indicated by the labels provided by the scRNA-seq reference dataset. As shown in the UMAP graph, about 8 cell types are clearly separated with the selected features. FIG. 6B predicts the conservative Signal Gain (cumulative), which is defined as the ratio of the panel signal to the highest gene signal, as a function of the number of genes. As shown in FIG. 6C, FISHnCHIPs signals are predicted to be 1.2 to 22.3-fold brighter than profiling with individual marker genes. FIG. 6C provides a module-cell expression heatmap, which are grouped into the 8 resolvable cell types. Using the gene-centric in situ hybridisation (ISH) cell characterisation method, an amplified signal can be obtained for each gene expression program.

FIG. 7 provides a schematic overview of an exemplary software pipeline to align, segment and cluster cell types based on the FISHnCHIPs imaging data obtained. To summarise, the stepwise data processing includes the following: 1) Input for the image processing workflow includes DAPI, FISHnCHIPS, and background (after 55% formamide wash) images; 2) Pre-processing segmentation of the images based on DAPI images to generate cell masks; 3) Registration and background subtraction of FISHnCHIPs images; 4) Generation of cell intensity matrix with a list of cell centroids using cell masks; 5) Clustering of the cell intensity matrix; 6) Output of the pipeline can be visualized in a heatmap, an UMAP, or a spatial map. The output generated from this pipeline can also be subjected to further analyses, such as classifications of spatial patterns and analysis of cell-cell interactions. The imaging results obtained from the in situ hybridisation method as described herein provides insides in cell types, cell-cell interactions, and spatial distributions of the cells within the tissue. Further processing of the imaging data is available and can be designed accordingly based on the purpose of the experiment.

FIG. 8 provides scatter plots of cell type abundances between three different repeated datasets, which demonstrates reliable reproducibility of the mouse brain FISHnCHIPs cell type profiling data among technical replicates.

FIG. 9 provides another example of the in situ hybridisation method as described herein, which is based on gene-centric FISHnCHIPs profiling of 20 gene expression programs in the mouse cortex. Instead of the gene-gene correlation matrix as demonstrated in FIG. 5, the correlated genes are identified based on a dimensionality reduction-based algorithm (consensus non-negative matrix factorization (NMF)) which infers coordinated gene expression in neurons. A gene-gene correlation analysis is performed on the 20 previously annotated gene expression programs, producing a FISHnCHIPs panel containing an average of 16 genes per program. The 20 neuronal gene expression programs (comprising 14 identity programs (ExcL2, ExcL3 . . . . Sub) and 6 activity programs (Erp, LrpD . . . Syn)) are detected by the FISHnCHIPs method as described herein and the resulting images are shown in FIG. 9A. FIG. 9A provides exemplary FISHnCHIPs images of a mouse brain tissue slice stained for programs ExcL2, ExcL5p3, ExcL6p1, ExcL6p2, IntSst, and IntPv out of the 20 programs used, with an average of 16 co-related genes imaged concurrently. Scale bar is 500 μm in all images. The identity programs appear more spatially localized while the activity programs are more ubiquitously expressed. Clustering analysis is conducted on 2,794 segmented single cells with the identity programs. FIG. 9B shows a heatmap of the mean fluorescence intensity per cell for each imaged program. As visualised in FIG. 9C by Uniform Manifold Approximation and Projection (UMAP), the cell-by-program intensity matrix is further clustered using the Louvain algorithm, resulting in 11 cell type clusters, each are labelled by the program annotations (L2/3, L3/4, L4/5 . . . , and Sub). FIG. 9D provides spatial maps of the detected cells within the tissue, separated by their cell types: L2/3 excitatory neurons (panel i), L3/4 excitatory neurons (panel ii), L4/5 excitatory neurons (panel iii), L5p1 excitatory neurons (panel iv), L5/6 excitatory neurons (panel v), L6p1 excitatory neurons (panel vi), IntPv inhibitory neurons (panel vii), IntSst inhibitory neurons (panel viii), IntNpy/CckVip inhibitory neurons (panel ix), hippocampus (panel x), and subiculum (panel xi). Scale bar for all images is 400 μm. The distribution of excitatory and inhibitory neurons along the cortical depth is further quantified. Quantification of the distribution of neuronal cells recapitulates the previous finding of the layered structural organisation of cells in the cortex. As demonstrated in FIG. 9E, the excitatory neurons are spatially organised as 6 distinct layers. The inhibitory neurons also display layer-specific localisations, according to FIG. 9F, with Npy and CckVip being more concentrated in the upper layers, whereas the Sst and Pv expressing neurons populated the deep layers. The example demonstrates that the present method can distinguish the neuronal subtypes that stratify the canonical laminar structure of the visual cortex. It is also demonstrated that the method used in identifying the gene module (gene expression program) is not limited to gene-gene correlation matrix as demonstrated in FIG. 5, but is also applicable to other methods of determining correlated genes.

FIG. 10 provides an evaluation of the gene-centric FISHnCHIPs panel of FIG. 9 in mouse visual cortex using a scRNA-seq reference dataset. As shown in FIG. 10A, the predicted conservative Signal Gain (cumulative), which is defined as the ratio of the panel signal to the highest gene signal, as a function of the number of genes, increases for all programs ranging from 1.2 to 7.6-folds. FIG. 10B is a scRNA-seq expression heatmap for the 20 gene expression programs. The heatmap visualises the predicted signals (rows normalized to the max, which is the sum of expression level for the co-regulated genes in the program) of the 20 gene expression programs. The heatmap provides an overview of the expression level of programs in different cell types (columns). As shown in FIG. 10B, the identity programs are expressed in a cell type specific manner (high specificity) and the activity programs are more ubiquitously expressed. FIG. 10C provides a Uniform Manifold Approximation and Projection (UMAP) representation of the 20 gene expression programs, labelled by the reference cell type annotations. The UMAP shows that cells from the same cell type are clustered close to each other. For example, the excitatory neurons are close together while the inhibitory/inter-neurons are well separated in clusters to the inhibitory neurons on the left of the UMAP. FIG. 10D provides simulated scRNA-seq feature plots of the 14 identify programs. Similar to FIG. 10B, which is a heatmap, FIG. 10D provides a visualisation of the program expression in light of cell types plotted in FIG. 10C. The evaluation of the exemplary gene-centric in situ hybridisation method as described herein shows amplified signal intensity (sensitivity)), while providing cell type specificity.

FIG. 11 shows the gradient formation of gene expression along the cortical depth of the mouse visual cortex as imaged by the gene-centric FISHnCHIPs panels of FIG. 9. FIG. 11A provides a heatmap of the FISHnCHIPS expression cell-by-program-intensity matrix, where the cells are ordered by their distance to the outer edge of the cortex. As defined in FIG. 9D, the cortical depth distance for each cell type is calculated based on the two white arcs. Based on the heatmap, some programs exhibit gradual intensity variation along the cortical depth. FIG. 11B provides a Uniform Manifold Approximation and Projection (UMAP) representation of the FISHnCHIPs feature plots of the 14 identity programs. These results suggest that the excitatory programs (except for ExcL6p1) varied continuously with distance to the outer edge of the cortex. Some programs had expression distributions that partially overlapped along the cortical depth, suggesting that spatial gene expression gradients could underlie the continuous neuronal sub-types. As demonstrated herein, the in situ hybridisation method can be used to uncover underlying structural patterns in tissue organization.

FIG. 12 demonstrates imaging of the mouse brain under lower magnifications using the in situ hybridisation method as described herein. FIG. 12A provides an overview of six different objective lenses used with their respective specification on magnification (M), numerical aperture (N.A.), and predicted light gathering power under epi-illumination configuration (F (epi)). The mean fluorescence intensity per cell is measured for Alexa594, Cy5, and IR800CW for the six different objective lenses as shown in FIG. 12B. Consistent among Alexa594, Cy5, and IR800CW, objective lenses with higher magnification is able collect signals at higher intensities. Within the same magnification level, water lenses can obtain images with higher signal intensity compared to air lenses. Exemplary unprocessed FISHnCHIPs images (one Field of View, FOV) of the mouse cortex are shown in FIG. 12C for the six different objective lenses (panels a-f). Signals above the background level are detected in cells labelled with FISHnCHIPs across all three-colour channels, even at lowest magnification of 10×, suggesting significantly improved signal intensity of the present method compared to conventional methods. FIG. 12D provides a quantification of the number of cells detected per Field of View (FOV) (n=5 FOVs, error bars indicate the standard deviation). Because of the wider field of view, the number of cells imaged was >~40 fold greater when using the 10× versus 60× objective lenses. The average number of cells detected for each lens is: 10× air: 3130, 10× water: 3088, 20× air: 1003, 20× water: 1041, 40×: 261, 60×: 73. With the improved signal, cells labelled with the method as described herein can be well detected under lower magnifications, thus enabling larger fields of view and more cells to be profiled in the same amount of time. To capture a larger number of cells, the 10× water objectives is later used for data acquisition in FIG. 13.

FIG. 13 demonstrates an exemplary gene-centric FISHnCHIPs profiling of 53 gene modules in the mouse brain under a large Field of View (FOV) (10× objective) of a whole tissue section. This allows coverage of a 36-fold larger area within the same amount of assay time (21 hrs) compared to 60× objective. Similar to the previous analysis, as shown in FIG. 13A, the unsupervised clustering of 54,834 cells is shown in the cell-by-module intensity matrix (FIG. 13A, left), which reveals 18 major cell types. As shown in the matrix, co-regulated gene modules are observed to be co-localized in the same cells and biologically related modules cluster closely in the expression space. A Uniform Manifold Approximation and Projection (UMAP) representation (FIG. 13A, right) for all cells is provided, with the separated clusters labelled accordingly. FIG. 13B provides individual spatial maps of the 18 distinct cell clusters in the large Field of View (FOV) in panels a-r: neurons 1, 2, 3, 4, 5, 6, 7, and 8, astrocytes, blood vessel associated cells, endothelial cells, ependymal cells, immature oligodendrocytes, mature oligodendrocytes 1 and 2, microglial, pericytes, and unknown cell types. Scale bar is 1000 μm. The profiling of cell types using the present gene-centric in situ hybridisation method under a low magnification demonstrates the enhanced signal sensitivity of the method as described herein, and provides a proof-of-concept for the profiling of cells within a tissue under a large Field of View (FOV), covering both neuronal and non-neuronal cell types.

FIG. 14 provides a simulation of gene-centric FISHnCHIPs panel using an exemplary unsorted scRNA-seq dataset to assess the clustering accuracy with respect to the reference annotations. FIG. 14A provides a scRNA-seq gene-gene correlation heatmap for the 674 feature genes from the mouse cortex library imaged in FIG. 13. The pair-wise Pearson's correlation coefficient of the feature genes is computed. Based on the correlation coefficient, the correlation matrix is clustered using the Leiden algorithm. The gene clusters resulted are further sub-clustered using hierarchical clustering into 53 gene modules, with a signal gain (SG) of about 1.9 to 20.2. FIG. 14B-FIG. 14E provides UMAP representation for cells in the scRNA-seq dataset predicted from different feature sets: FIG. 14B shows the prediction based on 1,000 highly variable genes. FIG. 14C shows the prediction based on 2,000 highly variable genes. FIG. 14D shows the prediction based on 3,000 highly variable genes. FIG. 14E shows the prediction based on 53 modules presented in FIG. 13. FIG. 14F shows the Adjusted Rand Index (ARI) of clustering cells at a resolution of 0.1 using FIG. 14B to FIG. 14E as features against the labels from the scRNA-seq dataset as ground truth. The 53-modules panel has an ARI score of 0.814, suggesting that it could recapitulate the known brain cell types to a large extent. For comparison, the ARI score with 1,000 highly variable genes (simulating a conventional assay profiling 1,000 genes individually) is only slightly higher at 0.846. Thus, the simulation shows that the in situ hybridisation method described herein provides amplified signal reading, while maintaining comparable profiling specificity compared to conventional assays.

FIG. 15 provides exemplary normalized images from the 53-modules FISHnCHIPs profiling under 10× objective lens, which covers 36-fold larger area in the same amount of assay time (21 hrs). For example, in FIG. 15A, gene module 39, gene module 41, gene module 53 are imaged using Alexa 594. FIG. 15B shows representative images of gene module 20, gene module 33, and gene module 36 using Cy5. FIG. 15C shows gene module 1, gene module 5, and gene module 6 using IRDye 800CW. The images are taken under 10× objective lens. Scale bar for all images is 1000 μm. Inserts are zoomed in region of the white box with the scale bar being 100 μm. These exemplary images display strong and well-resolved signals obtained using the method as described herein, despite the large Field of View (FOV) captured, demonstrating the enhancement in both imaging quality and efficiency of the present method.

FIG. 16 compares the cell types identified by FISHnCHIPS and the results of single-cell RNA sequencing (scRNA-seq). FIG. 16A provides a Uniform Manifold Approximation and Projection (UMAP) representation for frontal cortex cells from Harmony algorithm integration of the scRNA-seq reference and FISHnCHIPs data in composite. FIG. 16B provides Uniform Manifold Approximation and Projection (UMAP) representation for scRNA-seq cells with cell type labels provided by Saunders et. al. FIG. 16C shows the UMAP and labelling of the cells processed using the same FISHnCHIP method as described in FIG. 13. The UMAP representations show correspondence between the cell types identified by the in situ hybridisation method as described herein and scRNA-seq data.

FIG. 17 provides a sub-clustering analysis of the 53-module FISHnCHIPs data described in FIG. 13. FIG. 17A provides a FISHnCHIPs expression heatmap of the subtypes of blood vessel associated cells identified. FIG. 17B provides a FISHnCHIPs spatial map of the subtypes of blood vessel associated cells identified. FIG. 17C provides a Uniform Manifold Approximation and Projection (UMAP) of the subtypes of blood vessel associated cells identified. Various subtypes of cells are identified using the FISHnCHIPs experimental data. For example, distinct localisations for the subtypes of blood vessel associated cells, such as CNN1+ smooth muscle cells, DCN+ fibroblasts, MRC1+ (also known as CD206) border-associated macrophages that resided almost exclusively at the cortical surface, and GKN3+ arterial endothelial cells that formed large penetrating vascular structures are observed. Therefore, the in situ hybridisation method as described herein not only provide a profile for cell types, but also uncovers fine subtypes cells with distinct spatial distribution patterns.

FIG. 18 provides further validation of the performance of the high throughput FISHnCHIPS assay. Comparing the frequency and spatial distribution of cell types observed under 10× versus 60× objectives using two closely adjacent cryo-sections shows highly correlated cluster sizes between the 10× and 60× datasets (Pearson's correlation, r=0.95). FIG. 18A shows experimental datasets generated under 10× objectives, including plot showing all the segmented cells (panel a), filtered cells after removal of low expression cells in the first quality control stage (panel b), spatial map of cells after Leiden clustering (panel c), and Uniform Manifold Approximation and Projection (UMAP) representation of the clustering (panel d). FIG. 18B shows experimental datasets generated under 60× objectives, including plot showing all the segmented cells (panel e), filtered cells after removal of low expression cells in the first quality control stage (panel f), spatial map of cells after Leiden clustering (panel g), and Uniform Manifold Approximation and Projection (UMAP) representation of the clustering (panel h). Scale bar is 500 μm for both FIG. 18A and FIG. 18B. FIG. 18C provides a scatter plot of number of cells in each cluster detected by 60× versus 10×. Dash line represents the x=y line. This comparison indicates that no observable degradation of FISHnCHIPs data quality despite the increased throughput at lower magnification (such as 10×) compared to the higher magnification (such as 60×).

FIG. 19 demonstrates imaging of cancer associated fibroblasts (CAFs) subtypes using the in situ hybridisation method described herein. Two cancer-associated fibroblasts (CAFs) subtypes are imaged using the FISHnCHIPs method from a frozen biopsy of human colorectal cancer (CRC) tissue. The epithelial cells (labelled by tumor marker genes) and immune cells (labelled by human leukocyte antigen, HLA genes) in the CRC tissue are co-stained using FISHnCHIPs. FIG. 19A provides exemplary images of cancer associated fibroblasts 1 (CAF-1), cancer associated fibroblasts 2 (CAF-2), colon epithelium, and immune cells (HLA genes) in panels a to d, respectively. Scale bar is 200 μm. FIG. 19B provides in panels ii-v the zoomed-in region of the white box insert in composite panel i, with the scale bar being 25 μm. FIG. 19B in panels vi-viii shows the centroids of the segmented cell masks for CAF-1 (vi), CAF-2 (vii), and immune cells (viii). Scale bar is 200 μm. Box plots of the number of immune cells within 100 μm radius of CAF-1 (vi) and CAF-2 (vii) cells are shown in FIG. 19B. The number of cells in the box plot is: CAF-1:2,946 cells, CAF-2:2,671 cells. The box plot shows the median (centre line), the first and third quartiles (box limits), and 1.5× the interquartile range (whiskers). p=1.4×10⁻⁷², 2-sided Mann-Whitney U test. As shown in FIG. 19B, distinct spatial organization of the two CAF subtypes are observed. The CAF-2 subtype expressing the muscle contraction related genes appears to promote an immuno-suppressive microenvironment, where fewer immune cells (0.74-fold, p=1.4×10-72 (2-sided Mann-Whitney U test)) are detected in the vicinity of CAF-2 compared to CAF-1 subtypes. Immune cells were found 0.74-fold less frequently in the vicinity of CAF-2 than CAF-1. As demonstrated in this example, the in situ hybridisation method as described herein can characterize cells not only from healthy, but also from diseased tissue samples, such as cancer tissues. From the spatial organization information of the specific cell types within the tissue samples, additional insights related to the pathological development can be uncovered.

FIG. 20 provides an estimation of the signal gain (SG) for the human colorectal cancer (CRC) FISHnCHIPs panel of FIG. 19 for imaging cancer associated fibroblasts (CAFs) subtypes in human colorectal cancer (CRC) frozen biopsy tissue. FIG. 20A shows a scRNA-seq gene expression heatmap of the human colorectal cancer (CRC) FISHnCHIPs panel based on previously published information in Li, H. et al. (Li, H. et al. Reference component analysis of single-cell transcriptomes elucidates cellular heterogeneity in human colorectal tumors. Nat Genet 49, 708-718 (2017)). The reference SCRNA-seq data can be downloaded from Gene Expression Omnibus: EGAS00001001945/GSE81861. FIG. 20B shows a scRNA-seq gene expression heatmap of the human colorectal cancer (CRC) FISHnCHIPs panel based on a more recent scRNA-seq dataset published in Pelka et al. (Pelka, K. et al. Spatially organized multicellular immune hubs in human colorectal cancer. Cell 4734-4752 (2021).) FIG. 20C provides the predicted conservative signal gain (SG) for the human colorectal cancer (CRC) FISHnCHIPs panel, which shows significant signal gain for the detection of all four cell types. Clinical samples typically suffer from lower RNA quality, which limits the quality of the imaging of such samples. The use of genes that show coordinated changes in expression levels in the method as described herein results in high robustness and high signal gain, which facilitates the imaging of clinical samples.

FIG. 21 produces additional technical replicate of FISHnCHIPs on human colorectal cancer (CRC) tissue. FIG. 21A provides exemplary FISHnCHIPs image of CAF-1 subtype cells (panel a), CAF-2 subtype cells (panel b), colon epithelium (panel c), and immune cells (HLA genes) (panel d). The scale bar for all images in FIG. 21A is 250 μm. FIG. 21B shows composite FISHnCHIPs image of the four cell types in panel i. Scale bar is 250 μm. FIG. 21B under panels ii-v provides a zoom-in of the white box in panel i, with a scale bar showing 50 μm. FIG. 21B provides a box plot showing the number of immune cells within 100 μm radius of CAF-1 (vi) and CAF-2 (vii) cells. Consistent with the previous findings, immune cells were found 0.51-fold less frequently in the vicinity of CAF-2 subtype cells than CAF-1 subtype cells. The number of cells quantified in the box plot is: CAF-1:2,548 cells, CAF-2:2,199 cells. The box plots show the median (centre line), the first and third quartiles (box limits), and 1.5× the interquartile range (whiskers). p=8.5×10-142, 2-sided Mann-Whitney U test. Consistency in results of the in situ hybridisation imaging of cancer tissue demonstrates the reproducibility of the method as described herein.

FIG. 22 provides a three-colour immunofluorescence (IF) staining of the immune marker CD68, CAF-1 markers PDPN, LUM and PDGFA, and CAF-2 markers aSMA and MMP2 on four slices of frozen human colorectal cancer tissue. All images are contrasted at 1 to 99.9 percentiles of the maximum intensity of each channel. Scale bar is 250 μm in all images. The observed CAF-1 and CAF-2 patterns are in agreement with the immunofluorescence (IF) labelling, confirming the specificity and sensitivity of the present method.

FIG. 23 provides a two-colour single-molecule FISH (smFISH) staining of the CAF-1 markers DCN and MMP2, and CAF-2 markers ACTA2 and TAGLN at different concentrations on frozen human colorectal cancer tissue. DCN and TAGLN are stained together while MMP2 and ACTA2 are stained together on the same sample. SPARC single-molecule FISH staining for pan fibroblast is included as a positive control. Scale bar is 10 μm for all images. In contrast to the strong signals detected in FISHnCHIPs exemplified in FIG. 21, smFISH staining against DCN or MMP2 (markers for CAF-1), as well as TAGLN or ACTA2 (markers for CAF-2) are weaker and the CAFs subtypes re hardly distinguishable from the background noise. Therefore, the method as described herein which labels cell types based on multiple co-regulated genes are effective compared to conventional method such as single-molecule FISH in signal amplification.

FIG. 24 summarises the software workflow of the panel design and evaluation for both cell-centric and gene-centric strategies of the in situ hybridisation method as disclosed herein.

### DEFINITIONS

As used herein, the term “spatial transcriptomics” refers to molecular profiling method that allows measurement of all the gene activity (i.e. transcription) in a tissue and allows mapping of the location of the activity. Spatial transcriptomics comprises methods assigning cell types (identified by the mRNA readouts) to their locations in the histological sections. Methods commonly used in spatial transcriptomics includes fluorescent in situ hybridisation (FISH), in situ sequencing, in situ capture, and in silico construction.

As used herein, the term “hybridisation” refers to the formation of hybrid nucleic acid molecules with complementary nucleotide sequences. Hybridisation commonly happens between DNA and/or RNAs, in forms such as DNA: DNA, DNA: RNA, or RNA: RNA. Hybridisation process may happen naturally in vivo, for example, during DNA replication and transcription of DNA into RNA, or in vitro, such as during nucleic acid sequencing or a polymerase chain reaction (PCR).

As used herein, the term “in situ hybridisation” or “ISH” refers to an established, highly sensitive molecular biology technique that can be used to detect the presence or location of nucleic acids in preserved cells or tissue samples. This method is based on the complementary binding of a nucleotide probe to a specific target sequence of DNA or RNA. This technique can be further divided into two types based on the visualisation methods, i.e., fluorescence in situ hybridisation (FISH) or chromogenic in situ hybridisation (CISH).

As used herein, the term “fluorescence in situ hybridisation” or “FISH” refers to an in situ hybridisation visualized by a fluorescence signal. A typical fluorescence in situ hybridisation experiment requires a fluorescent copy of a probe sequence or a modified probe sequence that can be fluorescently tagged later. The probe sequence is designed such that it would be able to complementary bind to the specific target sequence. During hybridisation, the probe and the target chains are separated into single strands, for example, via heat or chemical to break the existing hydrogen bonds. The separated strands from the probe and the target are then allowed to reanneal via the complementary regions, forming new hydrogen bonds. After hybridisation, the probe may be visualized, for example, using a fluorescent microscope. There are other variations of fluorescence in situ hybridisation such as multiplex-FISH, spectral karyotyping, cross-species colour banding, and comparative genomic hybridisation which allows multi-colour imaging of the fluorescent signals. Single-molecule FISH (smFISH), also known as smRNA FISH or RNA FISH, can be used for imaging and quantifying of individual RNA molecules. Multiplexed error-robust FISH (MERFISH) is capable of simultaneously measuring the copy number and spatial distribution of large number of RNA species in single cells.

As used herein, the term “co-expression” or “co-expressed” are used to described genes that are expressed within the same cell, which implies that the genes are also expressed in very close spatial proximity within a tissue.

As used herein, the term “co-regulation” or “co-regulated” are used to describe genes that show coordinated changes in the gene expression level, i.e. covarying genes.

As used herein, the term “coordinated change”, “concordant change”, or “covarying” refers to consistency in changes to the gene expression level between two or more genes in the direction of change (increase or decrease) and timing. The term coordinated change refers to a positive correlation between the expression levels of the genes in a cell. For example, two or more genes may increase in expression level simultaneously, or decrease in expression level simultaneously. The magnitude of change can be coordinated as well. Correlation analysis is one way of identifying genes that are co-regulated or co-expressed. The default measure of correlation is the Pearson's correlation coefficient. The method of calculating such a correlation coefficient is well-established in the art. Besides Pearson's correlation coefficient, other possible methods of calculating the correlation coefficient include mutual information, Spearman's rank correlation coefficient, and Euclidean distance calculations. As used herein, the term “gene expression level” refers to the copy number of RNAs in a cell, or the level of transcription of RNAs from genes in a cell. The expression level of a gene within a cell is a combined result of both its synthesis and degradation. In the context of the present invention, “co-regulated” genes typically show coordinated changes in expression levels. This is because for eukaryotic transcription or RNA synthesis, co-regulated genes are likely to be co-transcribed, which may share common regulatory elements or mechanisms, such as transcription factors, enhancers, and repressors. For degradation, RNA copy number may be co-regulated by post-transcriptional mechanisms, such as miRNA.

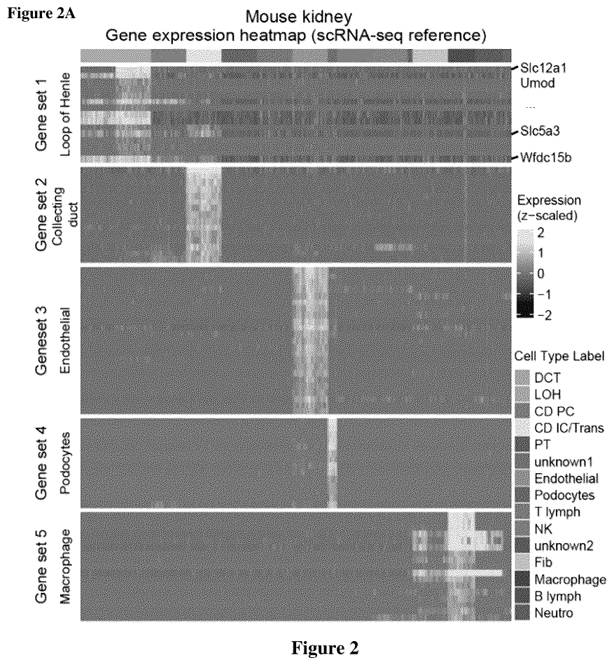

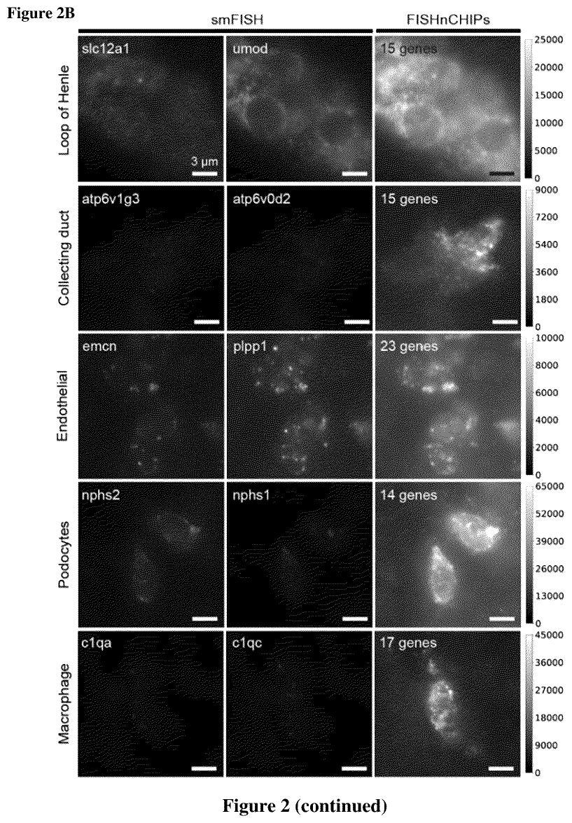

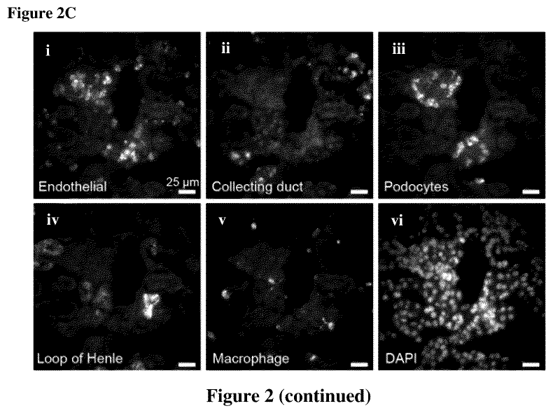

As used herein, the term “cell-centric” refers to a strategy of applying the in situ hybridisation method as described herein. As an initial step, the method requires user input of a list of marker genes defining a cell type. In a “cell-centric” strategy, the marker genes corresponded to a cell type of interest which are defined by the user. The definition can be based on existing information, such as information published in the literature or previous experimental observations. For example, as demonstrated in FIG. 2, five known cell-types are pre-defined when designing the panel to be used for in situ hybridisation (renal macrophages, glomerular endothelial cells, loop of Henle (LOH) cells, collecting duct (CD) cells, and glomerular podocytes). Alternative to a “cell-centric” strategy, a different “gene-centric” strategy of the method can be employed. As used herein, the term “gene-centric” in situ hybridisation refers to the method where the initial input is a set of thresholds/parameters to identify a set of genes with coordinated changes in their expression level, instead of a user definition of pre-determined genes defining a particular cell type. Such sets of genes can be “gene expression programs” or “gene modules”. Various data types (e.g. sequencing based Spatial Transcriptomics, sorted and unsorted scRNA-seq data) can also serve as references for the purpose of the method as described herein. The “gene-centric” strategy can be used to image multiple gene expression programs, and the collected signals can be further processed, for example, through quality control (QC), normalization and clustering to characterise the cells in a more unbiased manner. For example, as cell types can also be defined by the expression of multiple gene expression programs, through decoding of the collected “gene-centric” signals, a person skilled in the art can categorize the imaged cells into various cell types based on their expression profile.

As used herein, the terms “gene module”, “gene regulatory module” or “gene expression program” refers to a plurality of genes that shows a concordant change in their expression profiles under a given set of circumstances, such as the binding of the same set of transcription factors or co-factors. In the context of the method as described herein, the plurality of pre-determined genes shows coordinated changes in expression levels within a cell. These genes are biologically co-regulated, and can be, but are not limited to, markers of a specific cell type, differentially expressed genes of a specific cell type, markers of a gene expression program or gene regulatory module, or markers of a biological pathway. For example, “muscle contraction program” refers to a plurality of genes related to muscle contraction functions, and “neuronal program” refers to a plurality of genes related to neurons. Mechanisms such as action of cis/trans regulatory sequence, binding of non-coding RNAs, could be employed as “gene expression programs”. “Gene expression programs” can be obtained from skill of the art algorithms that identifies sets of genes with coordinated changes in their expression level. The clustering results of the gene-gene correlation matrix, for instance, is a “gene module” to be used as the input for the subsequent signal detection. The method for obtaining a “gene module” or “gene expression program” may include various unbiased approaches that are established in the art.

As used herein, the term “biological pathway” comprises of a set of protein/complex coding genes that interact with each other serially to initiate a biological process or form a certain product. Depending on database or literature, the number of genes within a ‘pathway’ is usually smaller than within a ‘module’. For example, in the Kyoto Encyclopedia of Genes and Genomes (KEGG) pathway annotation, “PATHWAY” is at a lower level than “MODULE”. For example, biological pathways can be derived from coordinated gene expression changes via gene-set enrichment analysis.

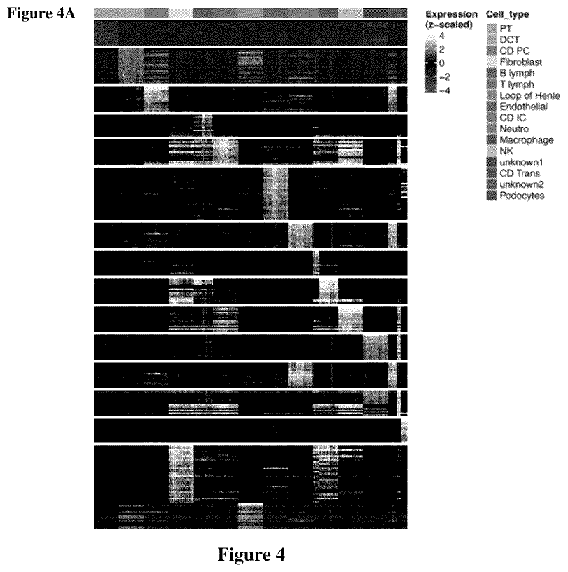

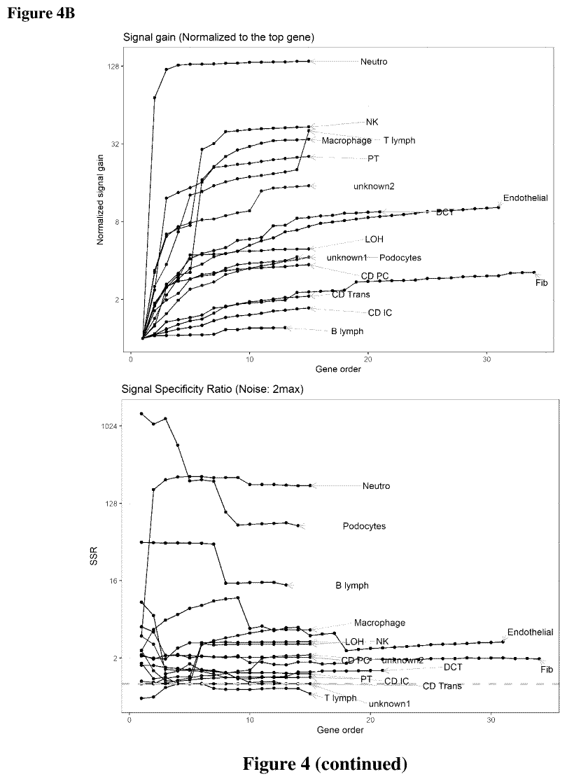

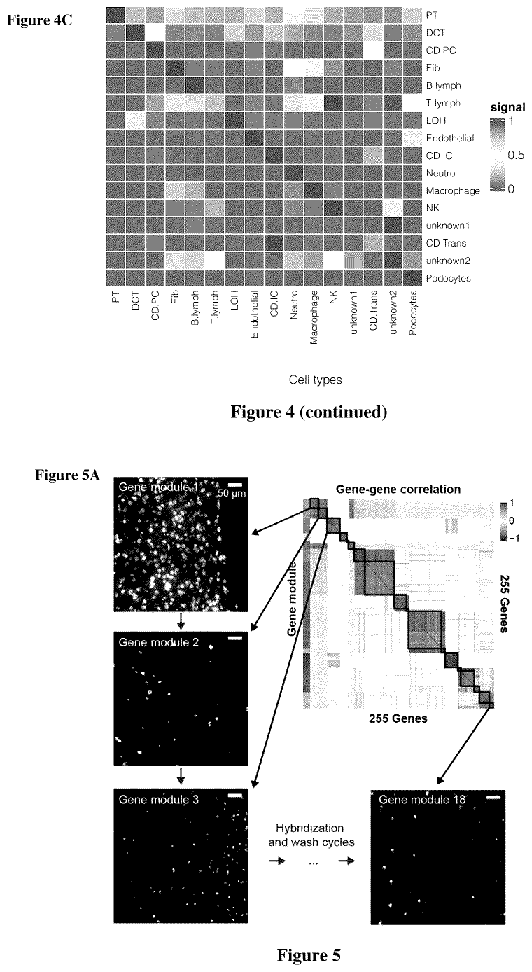

As used herein, the term “signal gain” or “SG” refers to the ratio of the sum of counts for the pre-determined target genes to that of the top differentially expressed genes. Signal gain quantifies the expected boost in signal when using the in situ hybridisation method as described herein versus conventional methods such as single-gene FISH. The SG metric can be easily interpreted. For example, if the predicted SG is 10, the cells labelled by the in situ hybridisation method are predicted to be tenfold brighter. In the kidney FISHnCHIPs experiment as described in FIG. 4, 4 out of 5 cell types have higher experimentally measured brightness than predicted. The minimum threshold should be decided upon by the user depending on the cases, while taking into account the signal specificity ratio threshold.

As used herein, the term “signal specificity ratio” or “SSR” refers to the ratio of the sum of counts for the pre-determined target genes in the target cell type to that in the most likely off-target cell type. Signal specificity ratio quantifies the predicted ‘noise’ when using the in situ hybridisation method as described herein versus conventional method such as single-gene FISH. When SSR approaches unity, the fluorescence intensity for the cell type of interest should be equal to that of an off-target cell type, rendering them indistinguishable. The SSR metric can be easily interpreted. For example, if the predicted SSR is 10, the target cells labelled by the in situ hybridisation method are predicted to be tenfold brighter than off-target cells. In the kidney FISHnCHIPs experiment described in FIG. 4, 5 out of 5 cell types have lower experimentally measured background noise than predicted. The minimum threshold should be decided upon by the user depending on the cases, while taking into account the SG threshold. It is emphasized that “SSR” and “SG” are predictive and are dependent on the quality of the input dataset.

As used herein, the term “Adjusted Rand Index” or “ARI” refers to a term that measures the similarity between two data clusterings. ARI is the is the corrected-for-chance version of the Rand index, which establishes a baseline by using the expected similarity of all pair-wise comparisons between clusterings specified by a random model. ARI can be used to quantify and compare the clustering accuracy when using the in situ hybridisation method as described herein versus conventional method such as single-gene FISH.

As used herein, the term “ground truth” refers to information that is known to be real or true, provided by direct observation or measurement (i.e. empirical evidence), as opposed to information provided by inference.

As used herein, the term “single-cell RNA sequencing” or “scRNA-seq” refers to the state-of-the-art sequencing approach which allows the detection of expression profiles of individual cells. Single-cell RNA sequencing uncovers the heterogeneity and complexity of RNA transcripts within single cells, as well as revealing the composition of different cell types and functions within highly organized tissues/organs/organisms.

As used herein, the term “pre-processing” refers to data preparation and manipulation on the raw input dataset

As used herein, the term “targeted” or “supervised” in the context of selecting marker genes refers to the selection of one or more genes based on prior knowledge of their expression level or biological specificity of the reference genes or markers. For example, the cell-centric strategy for the method described herein is a targeted method. In a targeted method, user needs to consider genome-wide gene co-expression to ensure the gene set of their selection is specific to the target cell types. In cases where an untargeted method does not produce specific markers or genes that matches prior knowledge or existing experimental results, the targeted approach may be used.

As used herein, the term “untargeted” or “unsupervised” in the context of selecting co-expressed genes refers to the selection of genes without prior knowledge of the expression level of said genes or the biological specificity of said genes. For example, the gene-centric strategy for the method described herein is an untargeted method. An “untargeted” or “unsupervised” selection of genes may allow clustering of cells based on inherent similarities of expression patterns without relying on prior known labels or categories. The untargeted method is suitable for tissues or samples that have little or no prior literature. Furthermore, an untargeted method has the potential to reveal cell types that are previously unknown.

As used herein, the term “identity program” refers to sets of genes that are collectively responsible for determining the identity or specialized function of a particular cell type or tissue in an organism.

As used herein, the term “activity program” refers to sets of genes that are turned on or off in response to specific environment cues or cellular signals.

As used herein, the term “detectable label” refers to a tag that allows distinguishing a tagged target being distinguished from untagged ones, typically through detection of visualized signals from the tag. A detectable label can be a protein, a nucleotide, or a chemical compound. Commonly used detectable labels include, for example, but are not limited to: fluorescent proteins, isotopes, mass tags. Fluorescent protein labelling is widely used in biological research in combination with imaging techniques, which allows the detection of the labelled targets in fixed or live samples. Visualisation of the fluorescent protein labels typically requires excitation by light at a particular wavelength range (excitation wavelength range), which allows the emission of detectable light at a different wavelength range (emission wavelength range). Collection of signals at an emission wavelength range allows visualisation of the fluorescent protein, thereby identifying the presence or absence, the location, and/or the quantity of the labelled target.

As used herein, the term “combination of emitted signals” refers to a collection of the emitted signals from a plurality of pre-determined genes having the same label or tags or similar label or tags emitting the same type of signal, which can be detected together via methods known in the art. In the context of the present disclosure, combined emitted signals of a set of pre-determined genes (for example, a gene module or a gene expression program) from the same fluorophore can be detected using fluorescence microscopy, using a single set of excitation and emission wavelengths. The detected signals would be a combination of all emitted signals from each of the tagged genes from the set of pre-determined genes, without distinguishing the signals from each individual gene.

As used herein, the term “plurality of emitted signals” refers to a collection of different signals emitted by a variety of detectable labels. In the context of the present disclosure, multiple gene modules or gene expression programs can be detectably labelled, each comprising a plurality of pre-determined genes. Every gene module or gene expression program can be labelled by a different type of label, such as fluorophore, which allows differentiation between different gene modules or gene expression programs when the emitted signals are measured. Within the gene module or gene expression programs, the individual genes are labelled using the same label, such as fluorophore. The “plurality of emitted signals” refers to the different signals emitted by the excited label from each gene module or gene expression program.

### DETAILED DESCRIPTION OF THE PRESENT INVENTION

High-throughput spatial characterisation of cells within intact biological samples has been a technical challenge. Existing methods often suffer from low efficiency, high costs, and poor scalability. To address these limitations, as described herein, the present disclosure provides an in situ hybridisation (ISH) method for cellular heterogeneity characterisation which enables accurate mapping of cell types without disrupting the tissue architecture.

The following detailed description is merely exemplary in nature and is not intended to limit the invention or the application and uses of the invention. Furthermore, there is no intention to be bound by any theory presented in the preceding background of the invention or the following detailed description.

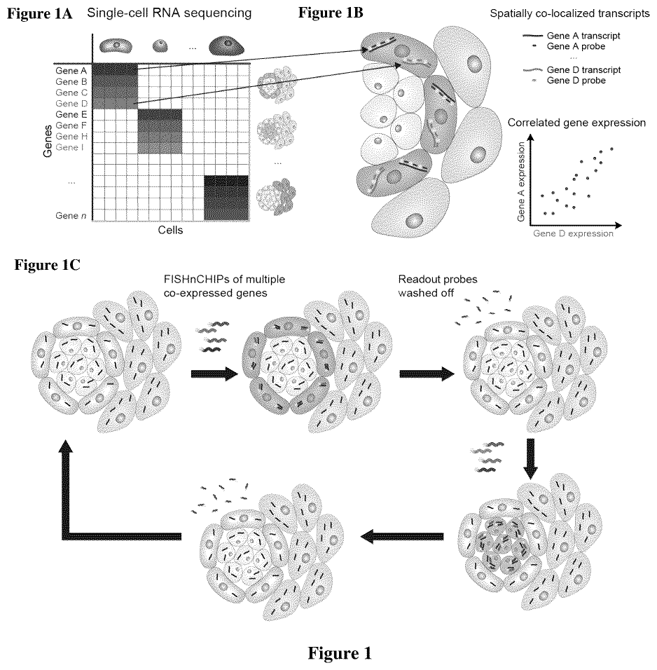

The present disclosure provides an in situ hybridisation (ISH) method which labels multiple genes simultaneously within specific cell types or molecular pathways, instead of a single gene, and measuring the collective signal emitted from these multiple genes within each cell. Targeting multiple genes results in a large number of detectable labels per cell (multiplication of transcript copy number per cell, number of probes per transcript, and number of genes targeted). Depending on the cell types or biological pathways of interest, the gain in signal is greater than 1, 10, 100, or 1000-folds, leading to more robustness and greater ease of detection. An overview of the method as described herein is shown in FIG. 1. Instead of focusing on accurate determination of the possible differentiation of single genes, the focus of this invention is to enhance the signal by adding signals of pre-determined genes which are related to each other by coordinated changes in expression level or co-variation (e.g. due to the fact that the pre-determined genes belong to the same pathway). These pre-determined genes can be detected together using the same detectable label (e.g. fluorophore), thereby amplifying the signals collected. As compared to conventional ISH methods which determine the attribution of each single gene to the overall signal, the method of the present invention utilizes the sum of the signals obtained from different pre-determined genes which allows improvement of the signal-to-noise ratio of the collected data.

The method as described herein is applicable to any cell population for which transcriptomic characteristics are known, thus allowing the interrogation of cell states not accessible by antibody-based methods. The method also allows to determine the spatial location of the enhanced cellular signal within a tissue or 3D cell cluster/formation, without disrupting the tissue architecture, thereby providing insights into spatial organization information of cells within a tissue.

The in situ hybridisation method described herein can be carried out through three major steps. A) designing panels of pre-determined genes or using sets of existing pre-determined genes to be targeted; B) labelling and imaging of the genes, and lastly, C) collection and processing of the collected data. Based on how the gene panels are designed, the in situ hybridisation method can be further sub-divided into two different strategies, i.e. cell-centric strategy and gene-centric strategy.

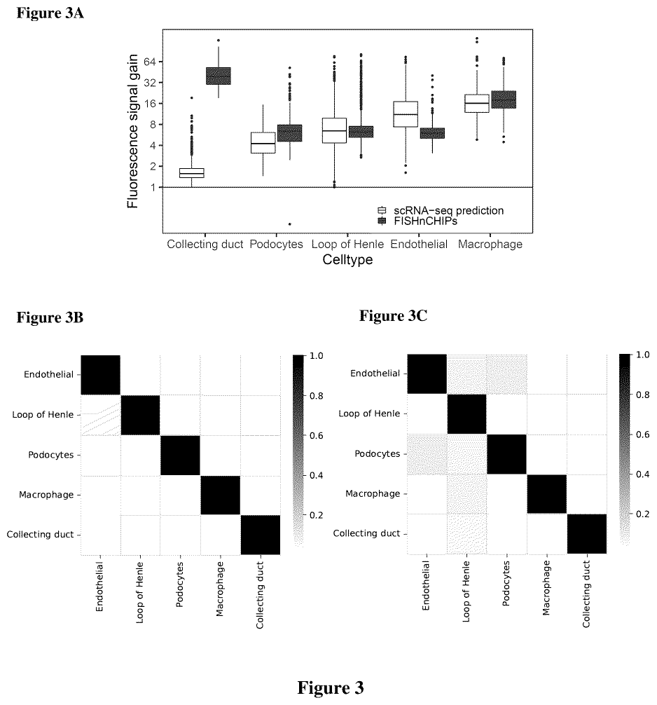

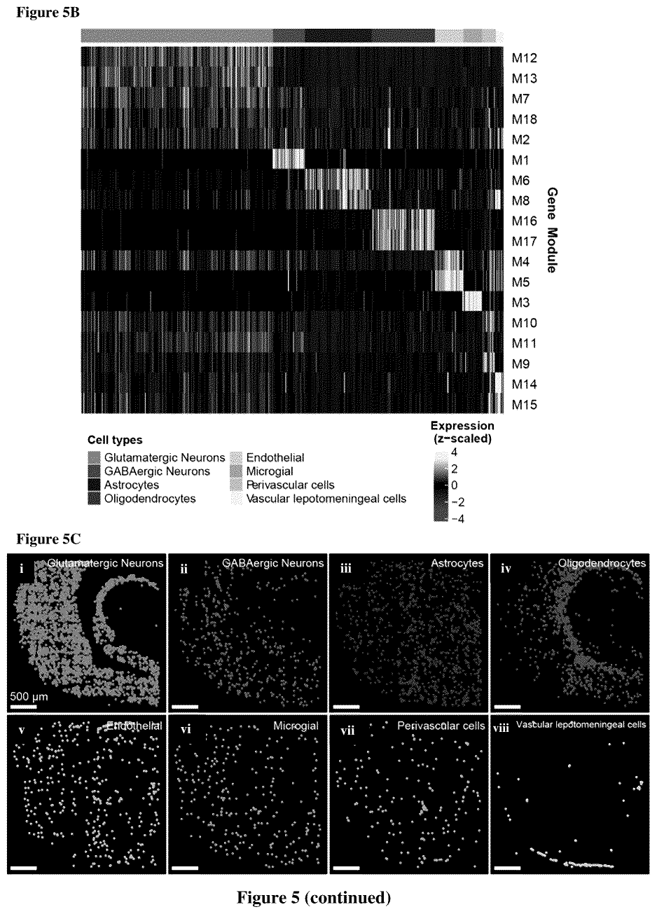

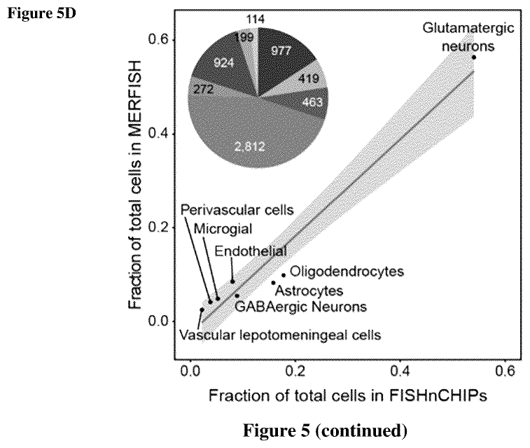

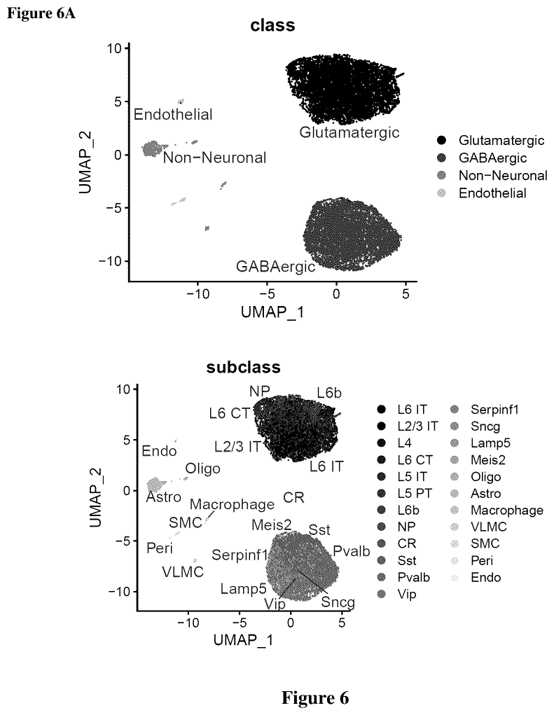

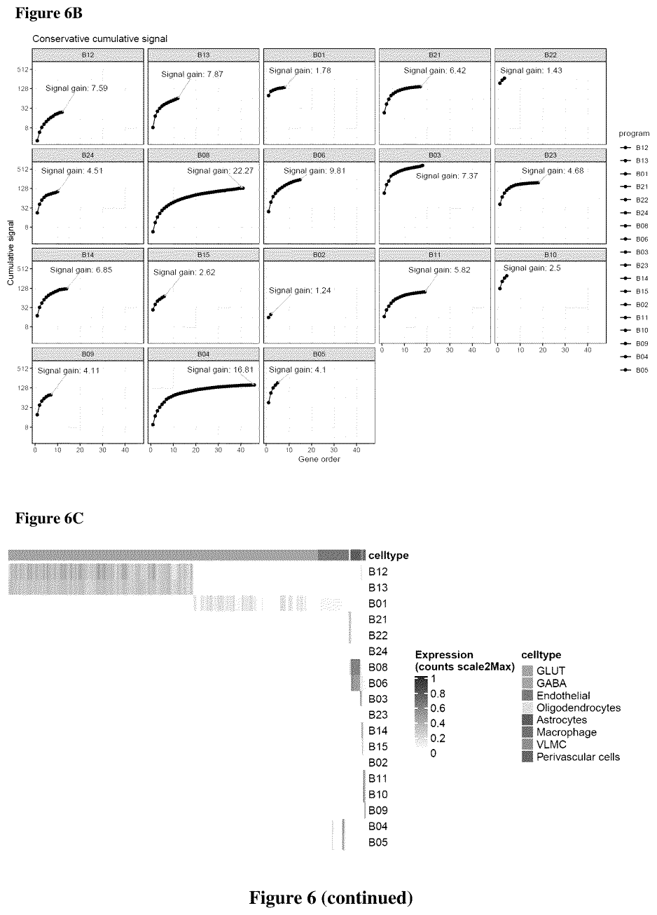

The present disclosure provides examples of both cell-centric and gene-centric strategies of the in situ hybridisation method. As exemplarily demonstrated in FIG. 2, a cell-centric FISH method is conducted for five selected cell types in mouse kidney. FIG. 5, for example, provides a gene-centric FISH method based on 18 gene modules in mouse cortex. Both strategies effectively profile the cell types within a tissue sample, showing consistent results with existing methods. Moreover, the method described herein shows increased signal intensity. In the cell-centric strategy, the fluorescence intensity per cell has increased by about 6 to 39-fold across the 5 cell types as shown in FIG. 3A. The signal gain in gene-centric strategy can be, according to FIG. 6C, about 1.2 to 22.3-fold brighter than profiling with individual marker genes. The workflows of the methods are briefly summarized as below.

### Cell-Centric In Situ Hybridisation (ISH) Strategy

1. Identifying a list of genes by calculating the expression co-variation of other genes with the reference cell type defining marker;
2. Designing ISH probes for the list of marker genes;
3. Evaluation of the ISH probe panel;
4. Exposing the cell samples to the probes and visualizing the probes after exposure;
5. Quantitation of the detectable signals obtained from the probes which bound to their target; and
6. Data analysis (such as clustering, cell-cell contact/proximity, tissue zonation) and presenting graphical data of cell clusters/heatmap.

### Gene-Centric In Situ Hybridisation (ISH) Strategy

1. Identifying sets of covarying genes (such as gene expression programs, gene modules, or pathways of interest) from a reference dataset or a database of interest;
2. Designing ISH probes for the sets of genes;
3. Evaluation of the ISH probe panel;
4. Exposing the cell samples to the probes and visualizing the probes after exposure;
5. Quantitation of the detectable signals obtained from the probes which bound to their target; and
6. Data analysis (such as clustering, cell-cell contact/proximity, tissue zonation) and presenting graphical data of cell clusters/heatmap.

As outlined above, one feature for the present disclosure will be the use of in situ hybridisation probes targeting single gene-set or multiple gene-sets (instead of single gene) that will be tagged by the same label, such as fluorophore, readout probe, or sequencing tag. Another feature for the present disclosure is the grouping of genes based on gene expression correlation to the cell type marker gene and clustering of the correlation matrix. Gene-gene correlation analysis is used, either across whole transcriptome or against cell-type marker genes, as an algorithmic approach to detect the above-mentioned gene-sets. Another technical feature of the present disclosure is the sequential hybridisation of multiple gene modules to allow de novo reconstruction of cell types in tissues.

Compared to conventional methods, the improved in situ hybridisation (ISH) method for cellular heterogeneity characterisation provides enhanced signal sensitivity. In one example, the sensitivity can be improved by about 2 to 200-fold (depending on the desired ‘cell type resolution’) compared to conventional in situ hybridisation methods. In another example, the sensitivity can be improved by about 20 to 200-fold. In another example, the signal sensitivity can be enhanced by at least 2 folds. In some examples, the signal sensitivity can be enhanced by at least about 5 folds, at least about 10 folds, at least about 20 folds, at least about 30 folds, at least about 40 folds, at least about 50 folds, at least about 60 folds, at least about 70 folds, at least about 80 folds, at least about 90 folds, or at least about 100 folds. In some examples, the signal sensitivity can be enhanced by about 2 to 20-fold, 20 to 100-fold, about 50 to 100-fold, or about 50 to 200-fold. In contrast to existing marker genes selection strategies that minimize redundancy or use compressed sensing to improve the multiplexing efficiency for individual genes, the method as described herein leverages the redundancy of correlated genes to boost sensitivity and robustness. For example, as shown in the box plot of FIG. 3A, the fluorescence signal gain per cell using the method described herewith is about 6 to 39-fold higher compared to conventional single-molecule FISH. In addition, the method as described herein reduces requirements in experimental equipment, experimental costs, and assay time. Large Field of View (FOV) imaging under low magnification can speed up the imaging process while retaining comparable imaging quality which is made possible due to the high signal-to-noise ratio even under low magnification (10×) as exemplarily shown in FIG. 13. Utilizing co-expressed genes, the in situ hybridisation method is also robust when analysing clinical tissues, which are typically characterized by low RNA quantity. Furthermore, optical crowding in small cells typically hinders the accurate decoding of highly-expressed RNA transcripts, but the method disclosed herein allows simultaneously profiling co-localized genes at the level of single cells. Compared to conventional multiplexed immunostaining methods, the method offers flexibility and throughput, as it exploits custom-designed and inexpensive oligonucleotide probes. Besides, labelling of antibody panels often requires individual optimization, but the detectable signal from the in situ hybridisation method described herein is more consistent because the efficiency of hybridisation of probes across the transcriptome.

Therefore, as described herein, the present disclosure provides a method of characterizing cells in a biological sample in situ.

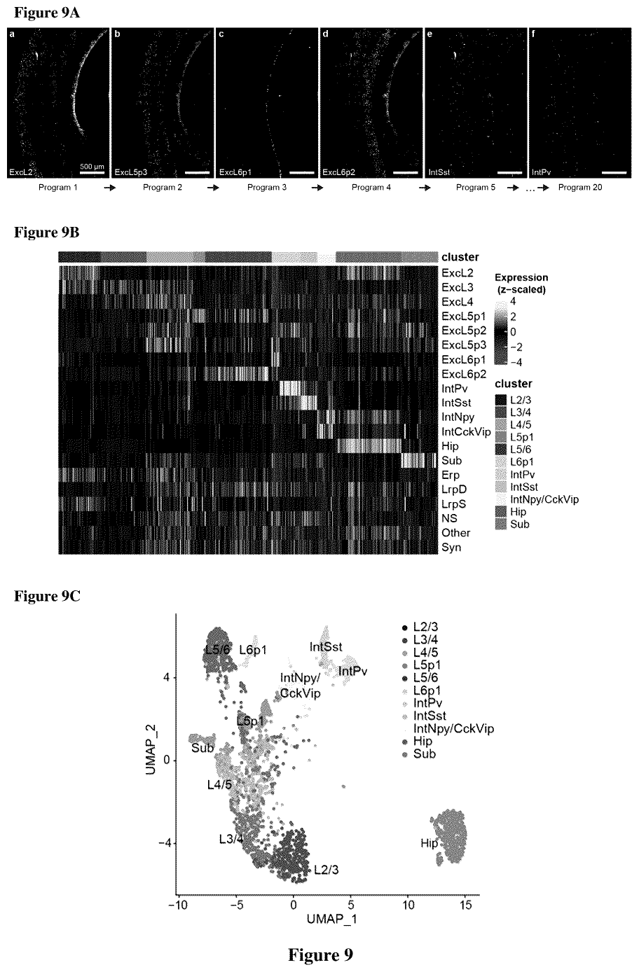

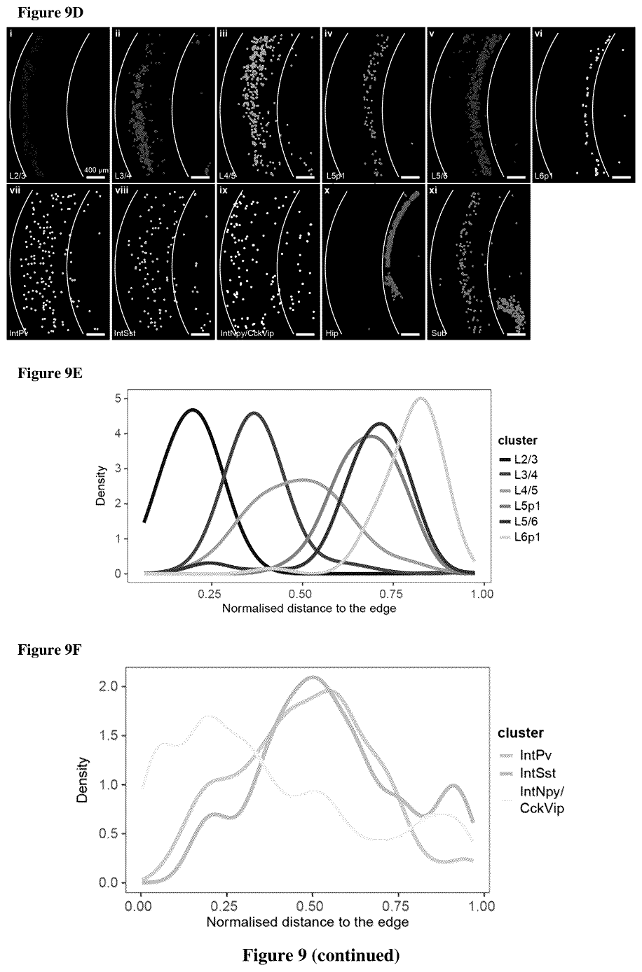

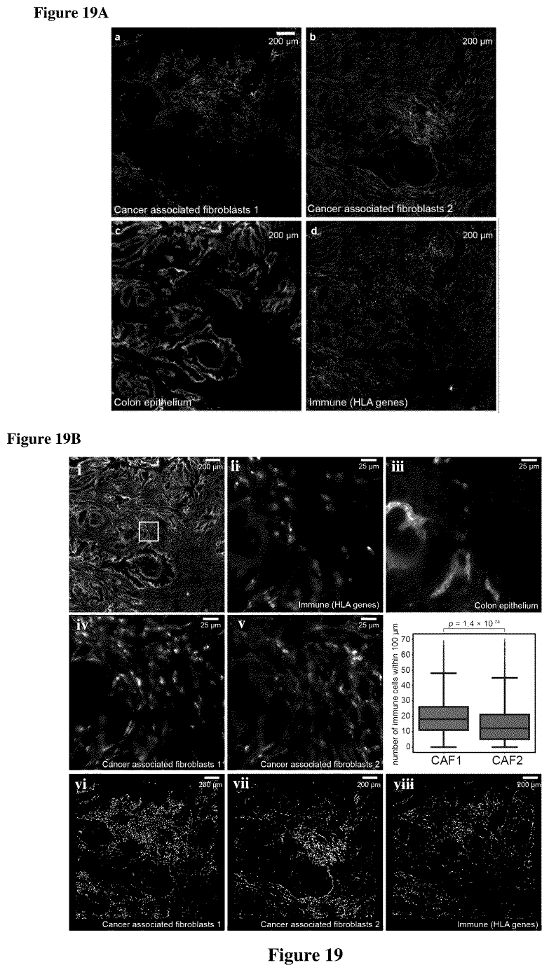

In one example, the method comprises contacting the biological sample with a plurality of probes that bind to ribonucleic acid (RNA) transcripts of a plurality of pre-determined genes. In one example, the method as described herein is an in vitro method. In another example, the method as described herein is conducted on a biological sample obtained from a subject. The biological sample can be, but is not limited to a tissue sample, a cultured sample (such as an in vitro or ex vivo sample, or an organoid), or a biopsy sample. The biological sample can be unprocessed (a fresh sample) or processed (for example, a fixed, frozen, embedded or tissue-cleared sample). In one example, the biological sample is fixed to or presented on an imaging slide, a cover slip, or a cell culture dish. In one specific example, the biological sample can be a Formalin-Fixed Paraffin-Embedded (FFPE) tissue, which typically suffers from having low quality of RNA which affects the labelling signal intensity. Signals from a FFPE tissue sample can be easily detected using the method as described herein due to the signal intensity compared to conventional methods as referred to above. In some cases, the biological sample comprises cells of the same tissue type. In some other cases, the biological sample comprises cells of different types. For example, as demonstrated in FIG. 13, an entire tissue section can be analyzed using the method described herein, which covers both neuronal and non-neuronal cell types. In other cases, FIG. 9 shows cell type profiling in mouse cortex covering only the neuronal cell types. Therefore, the biological sample can comprise a homogenous or heterogenous population of cells. In some examples, the biological sample can comprise healthy cells, or diseased cells, or both. FIG. 19 provides an example of imaging of cancer associated fibroblasts (CAFs) subtypes using the in situ hybridisation method described herein from a frozen biopsy of human colorectal cancer (CRC) tissue. In one example, the biological sample comprises cells that are adhered to a solid substrate. In another example, the biological sample is one of a plurality of samples within a tissue array, or one of a plurality of samples on a coverslip.

In one example, a probe as described herein is a probe made of a nucleic acid. The nucleic acid probe can be a ribonucleic acid (RNA) or a deoxyribonucleic acid (DNA). In another example, the probe as described herein comprises a nucleotide sequence. In another example, the probe comprises a domain that binds specifically to a ribonucleic acid transcript of one of the pre-determined genes. The binding between the probe and the target RNA transcript can be hybridisation, which is mediated by the formation of hydrogen bonds between complimentary nucleotides.

In on example, the selection of the plurality of pre-determined genes is an unsupervised selection, a supervised selection, or a combination of both. The unsupervised method is suitable for tissues or samples that have little or no prior literature. Furthermore, an unsupervised method has the potential to reveal cell types that are previously unknown. In cases where an unsupervised method does not produce specific markers or genes that matches prior knowledge or existing experimental results, the supervised approach may be used. In a supervised method, user needs to consider genome-wide gene co-expression to ensure the gene set of their selection is specific to the target cell types.

In one example, a plurality of pre-determined genes is targeted by the probes. The plurality of pre-determined genes comprises at least one gene and at least one other gene that show coordinated changes in expression levels. The method as described herein differs from conventional ISH methods, such as MERFISH, seqFISH, osmFISH, smFISH, or RNA scope because the method described herein uses probes to hybridise with the transcripts of multiple co-regulated gene targets (regulatory module/gene expression program) simultaneously, while the conventional methods label only one single target gene. The at least one, and at least one other pre-determined genes can include, but are not limited to markers of a specific cell type; differentially expressed genes of a specific cell type; markers of a gene expression program or gene regulatory module; markers of a biological pathways; or combinations thereof.

In a further example, the at least one other gene includes, but are not limited to, one or more input datasets such as: a bulk RNA sequencing, a single-cell RNA sequencing, a microarray dataset, a chromatin accessibility sequencing, a methylation sequencing, a DNA-associated proteins sequencing, a spatial transcriptomics sequencing, a multiplexed RNA fluorescence in situ hybridisation, a multiplexed immunohistochemistry, a bioinformatics database, or any user-defined dataset or combinations thereof. In another example, the bioinformatics database is selected from the group consisting of Kyoto Encyclopedia of Genes and Genomes (KEGG) or Panther or Database for Annotation, Visualization, and Integrated Discovery (DAVID) or Gene Ontology (GO) or combinations thereof. Additionally, prior knowledge on biochemical pathway, transcription factor motif, chromatin accessibility, bulk gene expression, sequencing-based spatial transcriptomics, or cis-regulatory sequences can be incorporated as part of the input. The in situ hybridisation method can be combined with split-probe, tissue clearing, or amplification to further enhance the signal. scRNA-seq methods and the availability of comprehensive cell atlas reference datasets can facilitate a wider array of cell types to be mapped using the method described herein.

Based on the input dataset, a person skilled in the art would be able to calculate, with existing mathematical tools, whether two genes are likely to show coordinated change in expression levels (i.e. co-regulated) within a cell, for example, through clustering of genes in a gene-gene correlation matrix, dimensionality reduction analysis (non-negative matrix factorization (NMF)), differential expression gene analysis or combinations thereof. The correlation, clustering, and dimensionality reduction analyses can be performed using mathematical analysis, such as Pearson's coefficient, mutual information, Spearman's correlation coefficient, Euclidean distance, non-negative matrix factorization, principle component analysis, Louvain or Leiden community detection algorithm, hierarchical-based, centroid-based clustering algorithm, or non-parametric Wilcoxon rank sum test.

In some examples, the co-regulated genes are further evaluated to identify the plurality of pre-determined genes. For example, the signal gain (SG) of the co-regulated genes is calculated to predict the expected improvement in signal intensity when using the method as described herein compared to conventional ISH methods. The signal gain (SG) is the ratio of the sum of the signals of the co-regulated genes to the signal of one gene, such as the differentially expressed gene or the gene with the highest expression. In some examples, the plurality of pre-determined genes is identified when the SG is above 1, 2, 5, 10, or 50. In another example, the signal specificity ratio (SSR) of the co-regulated genes is calculated to predict the (background) “noise” caused by off-target cell types in the signal generated when using the method as described herein compared to conventional ISH methods. The signal specificity ratio (SSR) is the ratio of the sum of the signals of the co-regulated genes in the target cells to the off-target cells or the cell cluster with the second highest expression. In some examples, the plurality of pre-determined genes is identified when the SSR is above 2, 5, 10, or 50. FIG. 4B provides an exemplary figure showing the calculated SG and SSR for the cell-centric FISHnCHIP experiment using signal reading in for the 5 cell types in mouse kidney.

In one example, the probes as described herein comprise a detectable label. In some examples, the detectable label can be directly detected. In other examples, the detectable label can be detected upon contacting it with one or more agents (sandwich labelling). In some examples, the detectable label is comprised in a separate readout probe. In one example, the detectable label is a fluorophore, a fluorescent protein, or a fluorescent dye. As described herein, the probe can emit a detectable signal upon binding to the target ribonucleic acid transcript, which allows detection of the signal. For example, when the signal is a fluorophore, the signal can be detected by exciting said fluorophore near its excitation maximum and observing fluorescence emission near its emission maximum. The resulting emission can be detected by an optical imaging instrument, such as a fluorescent microscope. Commonly used fluorophore colours include, but are not limited to: a) near-infrared; b) far-red; c) red; d) yellow; e) green; f) cyan; and g) blue. While some of the examples provided herein are based on fluorescence in situ hybridisation (FISH), it should be understood by a person skilled in the art that the same improved in situ hybridisation (ISH) method is compatible with other detection methods and detectable labels such as chromophores, radioisotopes, and chromogens.

Fluorescence labeled readout probes can be designed for transcriptome analysis in the improved fluorescence in situ hybridisation (FISH) method as described herein. The probes are tagged on the 5′ or the 3′ end. Exemplary sequences of the probe sequences and the tags are listed in Table 1 below:

TABLE 1
FISHnCHIPs Readout Probes

| Readout | | SEQ ID |
|---|---|---|
| ID | Probe Sequence | NO |
| B1 | /5IRD800CWN/GGTTCCAATCGGATC | 1 |
| B2 | /5IRD800CWN/CGAACGAACGATAGC | 2 |
| B3 | /5IRD800CWN/TCGGACGATCATGGG | 3 |
| B4 | /5IRD800CWN/ATTGACCGTCTCGTT | 4 |
| B5 | /5IRD800CWN/ATTAGGGCATCGACC | 5 |
| B6 | GCGCAGCAATTCACT/3Cy5Sp/ | 6 |
| B7 | GGTCCCGTTGAACTT/3Cy5Sp/ | 7 |
| B8 | /5IRD800CWN/AGCGCGTCAAACAGA | 8 |
| B9 | /5Alex594N/AACGAGCGTCCCTTG | 9 |
| B10 | /5Alex594N/CGTTGCGACGACTAA | 10 |
| B11 | CACCGTTGCGCTTAC/3Cy5Sp/ | 11 |
| B12 | /5Alex594N/TCCGTCACGCAATTT | 12 |
| B13 | /5Alex594N/CGTAGCGGAATCTGC | 13 |
| B14 | /5Alex594N/GTCGGGAACGGATAC | 14 |
| B15 | /5Alex594N/GATGTAATTCGGCCG | 15 |
| B17 | /5IRD800CWN/TATGTAAGTGGGTGG | 16 |
| B18 | TTAGGGAGGTGGGTG/3Cy5Sp/ | 17 |
| B19 | /5Alex594N/GTTAGGATGGGTTGT | 18 |
| B21 | /5IRD800CWN/GAAGGGAGTAATTGA | 19 |
| B22 | GGAGATGTTGTGAAG/3Cy5Sp/ | 20 |
| B23 | GTGATGTAGTGGGAT/3Cy5Sp/ | 21 |
| B24 | GAAGGAGTAGAGGAG/3Cy5Sp/ | 22 |
| B25 | CCTAAGGCAACGAGT/3Cy5Sp/ | 23 |
| B26 | ATGGACCTGCTCAGT/3Cy5Sp/ | 24 |
| B27 | TCATCCCTGTGCCAT/3Cy5Sp/ | 25 |
| B28 | AATGACGCAGACTCG/3Cy5Sp/ | 26 |
| B29 | CAATAGTCCAGTTCG/3Cy5Sp/ | 27 |
| B30 | CTAAGGTTCCCTCAG/3Cy5Sp/ | 28 |
| B31 | GATGCCTCCGTATCT/3Cy5Sp/ | 29 |
| B32 | GCAGAATGGTAAGGG/3Cy5Sp/ | 30 |
| B33 | /5IRD800CWN/TATGCTCACTCGCTG | 31 |
| B34 | /5IRD800CWN/CTGCGATACATTGTG | 32 |
| B35 | /5IRD800CWN/CCTACTGACACCGTA | 33 |
| B36 | /5IRD800CWN/GACAACCGTAAAGAG | 34 |
| B37 | /5IRD800CWN/ACAGTAGTGCCGTTG | 35 |
| B38 | /5IRD800CWN/GGAGCCCGTAAGTAT | 36 |
| B39 | /5IRD800CWN/ACCATCAATGCTCGT | 37 |
| B40 | /5IRD800CWN/CACCCTTGGGCTTAT | 38 |
| B41 | /5IRD800CWN/CCATTTGGCGTGAAG | 39 |
| B42 | /5IRD800CWN/GGAAGAGTGCTCATA | 40 |
| B43 | GAATGCGATGTGTCC/3AlexF594N/ | 41 |
| B44 | GCCTATGACAAGGAT/3AlexF594N/ | 42 |
| B45 | TAGCGAGAATCGTGG/3AlexF594N/ | 43 |
| B46 | CTCGCAATGTGACAA/3AlexF594N/ | 44 |
| B47 | TTGAGGTGCGAAGTC/3AlexF594N/ | 45 |
| B49 | TTCTGTCCTCGGTGA/3AlexF594N/ | 46 |
| B50 | CGTTCACGGCTGATA/3AlexF594N/ | 47 |
| B51 | CACTACGCTTGTGAC/3AlexF594N/ | 48 |
| B52 | AAATGTGTGGGCGAA/3AlexF594N/ | 49 |
| B53 | GTCCTCTGCTACAGT/3AlexF594N/ | 50 |
| B54 | AGGAGCAGTAGACAG/3Cy5Sp/ | 51 |
| B56 | /5IRD800CWN/GTAACCGAGTGGCAT | 52 |

In another example, the method comprises detecting a combination or plurality of emitted signals from the plurality of probes. The detection of a combination or plurality of emitted signals allows the amplification of detectable signals (factoring in the number of genes, transcript copy number per cell, and number of probes per transcript), which enhances the signal sensitivity for the method described herein at about 20 to 200-fold. In some examples, the level of the emitted signal detected can be quantified and/or processed based on the purpose of the experiment.

In some examples of the method as described herein, the step of contacting the biological sample with a plurality of probes, and the step of detecting a combination or plurality of emitted signals from the plurality of probes can be repeated one or more times using a plurality of probes that bind to RNA transcripts of a plurality of different pre-determined genes. This step assists to image multiple sets of a plurality of genes targeted by the probes within the same tissue, thereby allowing collection of multiple sets of data simultaneously.

In another example, the method further comprises characterizing the cells based on the combination of emitted signals or a plurality of emitted signals. A cell type can be defined by the expression profile of multiple gene regulatory modules (or gene expression programs). In some cases, the characterisation of the cells includes one or more of mapping the location of the cell in the biological sample; identifying an interaction between the cell and one or more other cells; identifying gene expression patterns of the cell in the biological sample and visualizing the spatial transcriptome of the cell in the biological sample; stratifying cancer subtypes to determine severity of cancer. Therefore, the in situ hybridisation method for cell heterogeneity characterisation as described herein can be used to capture the signal of multiple gene regulatory modules (or gene expression programs), or even genome wide, and the resulting signals can be further processed to reveal cell types in a more unbiased manner. In a further example, the characterisation of the cells comprises processing of the input dataset to improve the quality of the data. Methods of processing experimental data obtained from in situ hybridisation are known in the art. For example, the experimental data can be subject to a pre-processing process such as quality control (QC), normalization, log/linear transformation. The pre-processed data can be further analyzed by methods such as correlation analysis, clustering analysis, dimensionality reduction analysis, or differential expression gene analysis.

Therefore, as described herein, the present disclosure provides a method of characterizing cells in a biological sample in situ, comprising contacting the biological sample with a plurality of probes that bind to ribonucleic acid (RNA) transcripts of a plurality of pre-determined genes, wherein each probe comprises a detectable label, and a domain that binds specifically to a ribonucleic acid transcript of one of the pre-determined genes; wherein a signal is emitted when the probe binds to the ribonucleic acid transcript; detecting a combination or plurality of emitted signals from the plurality of probes; and characterizing the cells based on the combination or plurality of emitted signals, wherein the plurality of pre-determined genes comprises at least one gene and at least one other gene that are co-regulated within a cell. The method as described herein improves signal to noise ratio, reduces instrumentation requirements, and shortens experiment runtimes through grouping of multiple co-regulated genes and labelling them together. The method as described herein allows characterization of cells in a biological sample according to information based on cell type, cell subtype, and spatial localization of cells.

In a further example of the method described herein, the plurality of pre-determined genes is expressed in kidney, brain, digestive tract or combinations thereof. FIG. 2 provides an example of cell-centric cell type profiling in mouse kidney. Additionally, exemplary experimental data for cell type profiling in mouse brain cortex sample is shown in FIG. 5. FIG. 19 demonstrates gene-centric cell type profiling in a human colorectal tissue sample. While the exemplary data demonstrates use of the method as described herein in kidney, brain, and digestive tract, a person skilled in the art would understand that the method can be generally applied to other organs or tissue types. Besides, the method as described herein can be applied to any biological samples containing cells, and is not limited to the exemplified species including mouse and human.

In one example, the plurality of pre-determined genes is expressed in the kidney as shown in FIG. 2 to FIG. 4. In a further example, the genes are expressed specifically in cells of Loop of Henle, cells of collecting duct, endothelial cells, podocyte and macrophage cells of the kidney.

In one example, the plurality of pre-determined genes expressed in the podocyte include genes listed in Table 2 (2a). In another example, the plurality of pre-determined genes expressed in the endothelial cell include genes listed in Table 2 (2b). In another example, the plurality of pre-determined genes expressed in the Loop of Henle include genes listed in Table 2 (2c). In another example, the plurality of pre-determined genes expressed in the collecting duct include genes listed in Table 2 (2d). In another example, the plurality of pre-determined genes expressed in the macrophage cell include genes listed in Table 2 (2e).

TABLE 2
FISHnCHIPs for FIG. 2 Mouse Kidney Library

| Table ID | Gene | Transcript ID | Cell type |
|---|---|---|---|
| 2a | Nphs2 | ENSMUST00000027896.5 | Podocytes |
| | Nphs1 | ENSMUST00000006825.8 | Podocytes |
| | Clic3 | ENSMUST00000114265.4 | Podocytes |
| | Wt1 | ENSMUST00000139585.3 | Podocytes |
| | Cdkn1c | ENSMUST00000037287.6 | Podocytes |
| | Rab3b | ENSMUST00000003502.3 | Podocytes |
| | Shisa3 | ENSMUST00000087241.5 | Podocytes |
| | Sema3g | ENSMUST00000090180.2 | Podocytes |
| | Synpo | ENSMUST00000130044.1 | Podocytes |
| | Tmem54 | ENSMUST00000106064.5 | Podocytes |
| | Ddn | ENSMUST00000075444.6 | Podocytes |
| | Chst1 | ENSMUST00000065797.6 | Podocytes |
| | C1qtnf7 | ENSMUST00000121872.1 | Podocytes |
| | Rasl11a | ENSMUST00000031646.7 | Podocytes |
| 2b | Emcn | ENSMUST00000119475.1 | Endothelial |
| | Ppap2a | ENSMUST00000070951.6 | Endothelial |
| | Kdr | ENSMUST00000113516.1 | Endothelial |
| | Ehd3 | ENSMUST00000024860.7 | Endothelial |
| | Pi16 | ENSMUST00000114701.4 | Endothelial |
| | Egfl7 | ENSMUST00000145575.4 | Endothelial |
| | Eng | ENSMUST00000009705.9 | Endothelial |
| | Cd300lg | ENSMUST00000017453.7 | Endothelial |
| | Meis2 | ENSMUST00000102538.6 | Endothelial |
| | Nrp1 | ENSMUST00000026917.8 | Endothelial |
| | Dlc1 | ENSMUST00000033923.9 | Endothelial |
| | Cdh5 | ENSMUST00000034339.8 | Endothelial |
| | Ramp2 | ENSMUST00000129680.3 | Endothelial |
| | Ptprb | ENSMUST00000092167.5 | Endothelial |
| | Esam | ENSMUST00000002011.9 | Endothelial |
| | Fam167b | ENSMUST00000052835.8 | Endothelial |
| | Flt1 | ENSMUST00000031653.7 | Endothelial |
| | Hecw2 | ENSMUST00000087659.6 | Endothelial |
| | Mmrn2 | ENSMUST00000111908.1 | Endothelial |
| | AU021092 | ENSMUST00000050160.4 | Endothelial |
| | Pecam1 | ENSMUST00000103069.5 | Endothelial |
| | Cyyr1 | ENSMUST00000114174.2 | Endothelial |
| | Tmem204 | ENSMUST00000024984.6 | Endothelial |
| 2c | Slc12a1 | ENSMUST00000110495.2 | Loop of Henle |
| | Umod | ENSMUST00000033263.4 | Loop of Henle |
| | Cldn19 | ENSMUST00000084309.7 | Loop of Henle |
| | Cldn16 | ENSMUST00000161053.3 | Loop of Henle |
| | Ppp1r1b | ENSMUST00000078694.8 | Loop of Henle |
| | Sostdc1 | ENSMUST00000041407.5 | Loop of Henle |
| | Irx1 | ENSMUST00000077337.8 | Loop of Henle |
| | Egf | ENSMUST00000029653.2 | Loop of Henle |
| | Ppp1r1a | ENSMUST00000023133.6 | Loop of Henle |
| | Ptger3 | ENSMUST00000173533.1 | Loop of Henle |
| | Slc5a3 | ENSMUST00000113975.2 | Loop of Henle |
| | Tmem207 | ENSMUST00000165687.1 | Loop of Henle |
| | Shd | ENSMUST00000044216.6 | Loop of Henle |
| | Irx2 | ENSMUST00000074372.5 | Loop of Henle |
| | Wfdc15b | ENSMUST00000109376.4 | Loop of Henle |
| 2d | Atp6v1g3 | ENSMUST00000027643.5 | CD IC/Trans |
| | Atp6v0d2 | ENSMUST00000029900.5 | CD IC/Trans |
| | Foxi1 | ENSMUST00000060271.2 | CD IC/Trans |
| | Atp6v1c2 | ENSMUST00000095820.7 | CD IC/Trans |
| | Hepacam2 | ENSMUST00000183736.1 | CD IC/Trans |
| | Ociad2 | ENSMUST00000087195.5 | CD IC/Trans |
| | Slc26a4 | ENSMUST00000001253.7 | CD IC/Trans |
| | Guca2a | ENSMUST00000024015.2 | CD IC/Trans |
| | Rcan2 | ENSMUST00000177857.3 | CD IC/Trans |
| | Oxgr1 | ENSMUST00000058213.5 | CD IC/Trans |
| | Hmx2 | ENSMUST00000183219.3 | CD IC/Trans |
| | Insrr | ENSMUST00000029711.4 | CD IC/Trans |
| | Serpinb9 | ENSMUST00000006391.4 | CD IC/Trans |
| | Plet1 | ENSMUST00000114474.3 | CD IC/Trans |
| | Tmem117 | ENSMUST00000080141.4 | CD IC/Trans |
| 2e | C1qa | ENSMUST00000046285.5 | Macrophage |
| | C1qc | ENSMUST00000046332.5 | Macrophage |
| | C1qb | ENSMUST00000046384.8 | Macrophage |
| | H2-Aa | ENSMUST00000040655.8 | Macrophage |
| | H2-Eb1 | ENSMUST00000074557.9 | Macrophage |
| | H2-Ab1 | ENSMUST00000040828.5 | Macrophage |
| | Slamf9 | ENSMUST00000027830.4 | Macrophage |
| | P2ry6 | ENSMUST00000060174.4 | Macrophage |
| | Mgl2 | ENSMUST00000041550.7 | Macrophage |
| | Cd74 | ENSMUST00000050487.10 | Macrophage |
| | Aif1 | ENSMUST00000172693.3 | Macrophage |
| | Ms4a7 | ENSMUST00000067532.6 | Macrophage |
| | Cd72 | ENSMUST00000107926.3 | Macrophage |
| | Lilra5 | ENSMUST00000117550.1 | Macrophage |
| | Pf4 | ENSMUST00000031320.6 | Macrophage |
| | Fcgr4 | ENSMUST00000078825.4 | Macrophage |
| | Scimp | ENSMUST00000108534.4 | Macrophage |

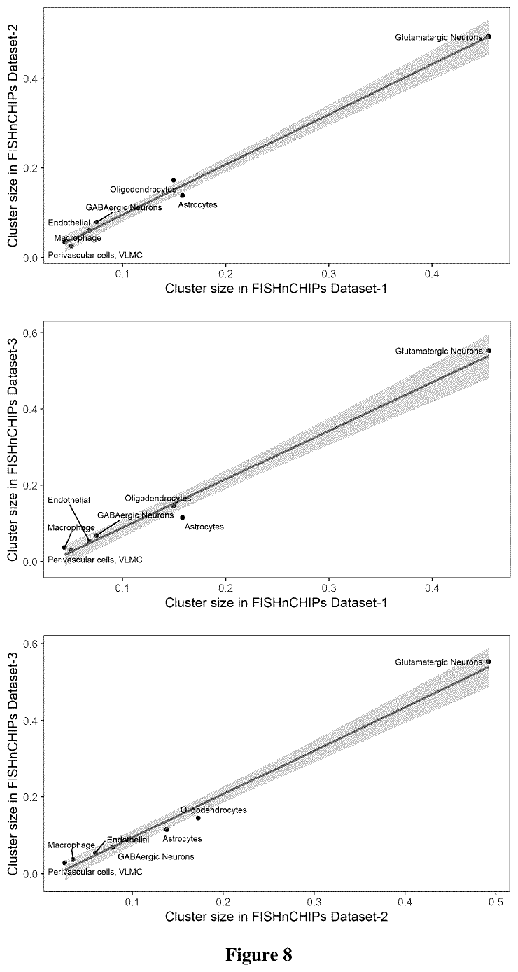

In one example, the plurality of pre-determined genes is expressed in neuronal tissues. In a further example, the pre-determined genes are expressed in brain cortex. FIG. 5 to FIG. 8 shows exemplary gene-centric profiling of 18 gene modules in mouse cortex.

In one further example, the plurality of pre-determined genes is expressed in a gene regulatory module in the brain, wherein said gene regulatory module is selected from M1, M2, M3, M4, M5, M6, M8, M9, M10, M11, M12, M13, M14, M15, M21, M22, M23 and M24. In another example, the plurality of pre-determined genes expressed in M1 include genes listed in Table 3 (3a). In another example, the plurality of pre-determined genes expressed in M2 include genes listed in Table 3 (3b). In another example, the plurality of pre-determined genes expressed in M3 include genes listed in Table 3 (3c). In another example, the plurality of pre-determined genes expressed in M4 include genes listed in Table 3 (3d). In another example, the plurality of pre-determined genes expressed in M5 include genes listed in Table 3 (3e). In another example, the plurality of pre-determined genes expressed in M6 include genes listed in Table 3 (3f). In another example, the plurality of pre-determined genes expressed in M8 include genes listed in Table 3 (3g). In another example, the plurality of pre-determined genes expressed in M9 include genes listed in Table 3 (3h). In another example, the plurality of pre-determined genes expressed in M10 include genes listed in Table 3 (3i). In another example, the plurality of pre-determined genes expressed in M11 include genes listed in Table 3 (3j). In another example, the plurality of pre-determined genes expressed in M12 include genes listed in Table 3 (3k). In another example, the plurality of pre-determined genes expressed in M13 include genes listed in Table 3 (31). In another example, the plurality of pre-determined genes expressed in M14 include genes listed in Table 3 (3m). In another example, the plurality of pre-determined genes expressed in M15 include genes listed in Table 3 (3n). In another example, the plurality of pre-determined genes expressed in M21 include genes listed in Table 3 (30). In another example, the plurality of pre-determined genes expressed in M22 include genes listed in Table 3 (3p). In another example, the plurality of pre-determined genes expressed in M23 include genes listed in Table 3 (3q). In another example, the plurality of pre-determined genes expressed in M24 include genes listed in Table 3 (3r).

TABLE 3
FISHnCHIPs for FIG. 5 Mouse Cortex Library

| Table | | | Gene |
|---|---|---|---|
| ID | Gene | Transcript ID | module |
| 3a | Vip | ENSMUST00000019906.4 | M1 |
| | Gad2 | ENSMUST00000028123.3 | M1 |
| | Slc6a1 | ENSMUST00000032454.5 | M1 |
| | Ap1s2 | ENSMUST00000069041.10 | M1 |
| | Rpp25 | ENSMUST00000080514.7 | M1 |
| | Igf1 | ENSMUST00000095360.6 | M1 |
| | Gad1 | ENSMUST00000130618.3 | M1 |
| | Dlx6os1 | ENSMUST00000159568.4 | M1 |
| 3b | Slc47a1 | ENSMUST00000010267.5 | M2 |
| | Car13 | ENSMUST00000029071.8 | M2 |
| 3c | Ly86 | ENSMUST00000021860.5 | M3 |
| | Trem2 | ENSMUST00000024791.10 | M3 |
| | Csf1r | ENSMUST00000025523.8 | M3 |
| | Tyrobp | ENSMUST00000032800.9 | M3 |
| | Cd53 | ENSMUST00000038845.9 | M3 |
| | C1qa | ENSMUST00000046285.5 | M3 |
| | C1qc | ENSMUST00000046332.5 | M3 |
| | C1qb | ENSMUST00000046384.8 | M3 |
| | Fcer1g | ENSMUST00000079957.7 | M3 |
| | Fcrls | ENSMUST00000090986.6 | M3 |
| | Gpr34 | ENSMUST00000096492.3 | M3 |
| | Selplg | ENSMUST00000100874.4 | M3 |
| | Ctss | ENSMUST00000116304.2 | M3 |
| | Laptm5 | ENSMUST00000151698.3 | M3 |
| | Fcgr3 | ENSMUST00000164044.3 | M3 |
| | P2ry12 | ENSMUST00000170388.1 | M3 |
| | Siglech | ENSMUST00000173835.1 | M3 |
| | Cx3cr1 | ENSMUST00000177637.1 | M3 |
| 3d | Icam2 | ENSMUST00000001055.10 | M4 |
| | Eng | ENSMUST00000009705.9 | M4 |
| | Gata2 | ENSMUST00000015197.7 | M4 |
| | Srgn | ENSMUST00000020271.8 | M4 |
| | Degs2 | ENSMUST00000021691.4 | M4 |
| | Ctla2a | ENSMUST00000021880.9 | M4 |
| | Ocln | ENSMUST00000022140.7 | M4 |
| | Podxl | ENSMUST00000026698.7 | M4 |
| | Slc16a4 | ENSMUST00000029502.9 | M4 |
| | Lef1 | ENSMUST00000029611.9 | M4 |
| | Nos3 | ENSMUST00000030834.6 | M4 |
| | Anxa3 | ENSMUST00000031447.7 | M4 |
| | Flt1 | ENSMUST00000031653.7 | M4 |
| | Pglyrp1 | ENSMUST00000032573.6 | M4 |
| | Slc38a5 | ENSMUST00000033512.6 | M4 |
| | Cdh5 | ENSMUST00000034339.8 | M4 |
| | Id1 | ENSMUST00000038368.8 | M4 |
| | Nostrin | ENSMUST00000041865.7 | M4 |
| | Foxq1 | ENSMUST00000042118.9 | M4 |
| | Tgtp2 | ENSMUST00000046745.6 | M4 |
| | Eltd1 | ENSMUST00000046977.7 | M4 |
| | Tie1 | ENSMUST00000047421.5 | M4 |
| | AbcM1a | ENSMUST00000047753.4 | M4 |
| | AU021092 | ENSMUST00000050160.4 | M4 |
| | Sox18 | ENSMUST00000054491.5 | M4 |
| | Sp100 | ENSMUST00000066427.6 | M4 |
| | Slfn5 | ENSMUST00000067443.4 | M4 |
| | Klf2 | ENSMUST00000067912.7 | M4 |
| | Tgtp1 | ENSMUST00000068063.3 | M4 |
| | St6galnac2 | ENSMUST00000079545.5 | M4 |
| | Ptprb | ENSMUST00000092167.5 | M4 |
| | Thbd | ENSMUST00000099270.4 | M4 |
| | AW112010 | ENSMUST00000099676.4 | M4 |
| | Tek | ENSMUST00000102798.3 | M4 |
| | Robo4 | ENSMUST00000102895.4 | M4 |
| | Pecam1 | ENSMUST00000103069.5 | M4 |
| | Klf4 | ENSMUST00000107619.2 | M4 |
| | Lsr | ENSMUST00000108116.5 | M4 |
| | Kdr | ENSMUST00000113516.1 | M4 |
| | Gpr116 | ENSMUST00000113599.1 | M4 |
| | Paqr5 | ENSMUST00000113990.1 | M4 |
| | Cyyr1 | ENSMUST00000114174.2 | M4 |
| | Acvrl1 | ENSMUST00000119063.3 | M4 |
| | Emcn | ENSMUST00000119475.1 | M4 |
| | Fn1 | ENSMUST00000186129.2 | M4 |
| | Sox17 | ENSMUST00000191939.1 | M4 |
| 3e | Cldn5 | ENSMUST00000043577.1 | M5 |
| | Pltp | ENSMUST00000059954.9 | M5 |
| | Ly6c1 | ENSMUST00000065408.11 | M5 |
| | Slco1a4 | ENSMUST00000165990.3 | M5 |
| | Ly6a | ENSMUST00000187994.2 | M5 |
| 3f | Btbd17 | ENSMUST00000000206.3 | M6 |
| | Sox9 | ENSMUST00000000579.2 | M6 |
| | Slc7a10 | ENSMUST00000001854.7 | M6 |
| | Grin2c | ENSMUST00000003351.8 | M6 |
| | Prodh | ENSMUST00000003620.7 | M6 |
| | Cyp4f15 | ENSMUST00000008801.6 | M6 |
| | Mertk | ENSMUST00000014505.4 | M6 |
| | Pdlim4 | ENSMUST00000018755.5 | M6 |
| | Fabp7 | ENSMUST00000020024.7 | M6 |
| | Fam20a | ENSMUST00000020938.7 | M6 |
| | Slc9a3r1 | ENSMUST00000021077.3 | M6 |
| | Timp4 | ENSMUST00000032462.6 | M6 |
| | Slc27a1 | ENSMUST00000034267.4 | M6 |
| | Oaf | ENSMUST00000034512.5 | M6 |
| | Acsbg1 | ENSMUST00000034822.7 | M6 |
| | Fam107a | ENSMUST00000036070.10 | M6 |
| | Cmtm5 | ENSMUST00000037814.6 | M6 |
| | Lcat | ENSMUST00000038896.7 | M6 |
| | Mlc1 | ENSMUST00000042594.8 | M6 |
| | Hepacam | ENSMUST00000051839.7 | M6 |
| | Dbx2 | ENSMUST00000054244.6 | M6 |
| | Cyp2j9 | ENSMUST00000055693.8 | M6 |
| | Tst | ENSMUST00000058659.7 | M6 |
| | S100a1 | ENSMUST00000060738.8 | M6 |
| | Fgfr3 | ENSMUST00000067150.9 | M6 |
| | Cbs | ENSMUST00000067801.8 | M6 |
| | Aqp4 | ENSMUST00000079081.6 | M6 |
| | Slc39a12 | ENSMUST00000082290.7 | M6 |
| | Dio2 | ENSMUST00000082432.3 | M6 |
| | Ppp1r3c | ENSMUST00000087321.2 | M6 |
| | S100a16 | ENSMUST00000098911.5 | M6 |
| | Nkain4 | ENSMUST00000103053.5 | M6 |
| | Slc25a18 | ENSMUST00000112682.2 | M6 |
| | Plcd4 | ENSMUST00000113747.3 | M6 |
| | Tlcd1 | ENSMUST00000127587.3 | M6 |
| | Tril | ENSMUST00000127748.3 | M6 |
| | Ppp1r3g | ENSMUST00000132661.1 | M6 |
| | Tsc22d4 | ENSMUST00000141733.3 | M6 |
| | Cml1 | ENSMUST00000161198.2 | M6 |
| | Il18 | ENSMUST00000180021.1 | M6 |
| | Slc38a3 | ENSMUST00000193932.1 | M6 |
| 3g | Gstm1 | ENSMUST00000004140.6 | M8 |
| | Slc1a3 | ENSMUST00000005493.9 | M8 |
| | Pla2g7 | ENSMUST00000024706.7 | M8 |
| | Gpr37l1 | ENSMUST00000027682.8 | M8 |
| | F3 | ENSMUST00000029771.8 | M8 |
| | Slco1c1 | ENSMUST00000032362.9 | M8 |
| | GjM6 | ENSMUST00000039380.8 | M8 |
| | S1pr1 | ENSMUST00000055676.2 | M8 |
| | Ppap2b | ENSMUST00000064139.7 | M8 |
| | Gja1 | ENSMUST00000068581.7 | M8 |
| | Atp1a2 | ENSMUST00000085913.6 | M8 |
| | Bcan | ENSMUST00000090971.6 | M8 |
| | Cldn10 | ENSMUST00000100314.3 | M8 |
| | Mfge8 | ENSMUST00000107409.3 | M8 |
| | Ntsr2 | ENSMUST00000111064.1 | M8 |
| 3h | Crip1 | ENSMUST00000006523.7 | M9 |
| | Tagln | ENSMUST00000034590.2 | M9 |
| | Acta2 | ENSMUST00000039631.8 | M9 |
| | Myl9 | ENSMUST00000088552.6 | M9 |
| 3i | Cnn1 | ENSMUST00000001384.4 | M10 |
| | KcnmM1 | ENSMUST00000020362.2 | M10 |
| | Aspn | ENSMUST00000021820.8 | M10 |
| | Mylk | ENSMUST00000023538.8 | M10 |
| | Des | ENSMUST00000027409.9 | M10 |
| | Mcam | ENSMUST00000034650.10 | M10 |
| | Wtip | ENSMUST00000038537.8 | M10 |
| | Mustn1 | ENSMUST00000040715.6 | M10 |
| | HspM2 | ENSMUST00000042790.3 | M10 |
| | Lmod1 | ENSMUST00000059352.2 | M10 |
| | Olfr78 | ENSMUST00000060187.9 | M10 |
| | Ptrf | ENSMUST00000060792.5 | M10 |
| | Gpr20 | ENSMUST00000064166.4 | M10 |
| | Rasl12 | ENSMUST00000085453.4 | M10 |
| | Myh11 | ENSMUST00000090287.3 | M10 |
| | Aoc3 | ENSMUST00000103105.5 | M10 |
| | Slc38a11 | ENSMUST00000112420.3 | M10 |
| | Myom1 | ENSMUST00000179759.1 | M10 |
| | Mir143hg | ENSMUST00000182244.3 | M10 |
| 3j | Cox4i2 | ENSMUST00000010020.7 | M11 |
| | Higd1b | ENSMUST00000021302.10 | M11 |
| | Kcnj8 | ENSMUST00000032374.7 | M11 |
| | Ndufa4l2 | ENSMUST00000035735.9 | M11 |
| | Atp13a5 | ENSMUST00000075806.6 | M11 |
| | P2ry14 | ENSMUST00000091112.4 | M11 |
| | Art3 | ENSMUST00000128246.3 | M11 |
| 3l | Stx1a | ENSMUST00000005509.6 | M12 |
| | Ptk2b | ENSMUST00000022622.9 | M12 |
| | Pcsk2 | ENSMUST00000028905.9 | M12 |
| | Nrn1 | ENSMUST00000037623.10 | M12 |
| | Neurod6 | ENSMUST00000044767.8 | M12 |
| | Ctxn1 | ENSMUST00000053252.7 | M12 |
| | Nrgn | ENSMUST00000065668.7 | M12 |
| | Baiap2 | ENSMUST00000075180.7 | M12 |
| | Slc17a7 | ENSMUST00000085374.5 | M12 |
| | 3110035E14Rik | ENSMUST00000088666.3 | M12 |
| | Rasgrp1 | ENSMUST00000102534.6 | M12 |
| | Arpp21 | ENSMUST00000162065.3 | M12 |
| 3m | Kcnv1 | ENSMUST00000022967.5 | M13 |
| | Itpka | ENSMUST00000028758.7 | M13 |
| | Egr3 | ENSMUST00000035908.1 | M13 |
| | Ier5 | ENSMUST00000055322.5 | M13 |
| | Rprml | ENSMUST00000057870.3 | M13 |
| | Rtn4r | ENSMUST00000059589.5 | M13 |
| | Fam212b | ENSMUST00000066610.7 | M13 |
| | Sv2b | ENSMUST00000085164.5 | M13 |
| | Cnksr2 | ENSMUST00000112513.1 | M13 |
| | Lingo1 | ENSMUST00000114247.1 | M13 |
| | Fmnl1 | ENSMUST00000129726.2 | M13 |
| | Mkl2 | ENSMUST00000149359.1 | M13 |
| 3n | Igf2 | ENSMUST00000000033.7 | M14 |
| | Col1a1 | ENSMUST00000001547.7 | M14 |
| | Slc22a6 | ENSMUST00000010250.2 | M14 |
| | Ogn | ENSMUST00000021822.5 | M14 |
| | Col1a2 | ENSMUST00000031668.8 | M14 |
| | Slc13a4 | ENSMUST00000031868.4 | M14 |
| | Aldh1a2 | ENSMUST00000034723.5 | M14 |
| | Lum | ENSMUST00000038160.4 | M14 |
| | Aox3 | ENSMUST00000040999.9 | M14 |
| | Fmod | ENSMUST00000048183.7 | M14 |
| | Fam180a | ENSMUST00000051176.7 | M14 |
| | GjM2 | ENSMUST00000055698.7 | M14 |
| | Slc6a13 | ENSMUST00000064580.9 | M14 |
| | Bmp6 | ENSMUST00000171970.1 | M14 |
| 3o | Serping1 | ENSMUST00000023994.5 | M15 |
| | Pcolce | ENSMUST00000031731.9 | M15 |
| | Bgn | ENSMUST00000033741.10 | M15 |
| | Colec12 | ENSMUST00000040069.8 | M15 |
| | Slc6a20a | ENSMUST00000040960.8 | M15 |
| | Dcn | ENSMUST00000105287.5 | M15 |
| 3p | Hapln2 | ENSMUST00000005014.4 | M21 |
| | Aspa | ENSMUST00000021119.4 | M21 |
| | Car14 | ENSMUST00000036181.10 | M21 |
| | Fa2h | ENSMUST00000038475.8 | M21 |
| | Cldn11 | ENSMUST00000046174.7 | M21 |
| | Gpr37 | ENSMUST00000054867.6 | M21 |
| | Ugt8a | ENSMUST00000057944.7 | M21 |
| | Gjc3 | ENSMUST00000077119.6 | M21 |
| | Opalin | ENSMUST00000087176.6 | M21 |
| | Myrf | ENSMUST00000088013.7 | M21 |
| | Ermn | ENSMUST00000090940.5 | M21 |
| | Tmem88b | ENSMUST00000097742.2 | M21 |
| | Nkx6-2 | ENSMUST00000097974.4 | M21 |
| | Mog | ENSMUST00000102665.6 | M21 |
| | GjM1 | ENSMUST00000119190.1 | M21 |
| | 1700047M11Rik | ENSMUST00000189594.1 | M21 |
| | Mag | ENSMUST00000190638.2 | M21 |
| 3q | Mal | ENSMUST00000028854.10 | M22 |
| | Plp1 | ENSMUST00000113085.1 | M22 |
| | Mobp | ENSMUST00000174193.3 | M22 |
| | Ccl24 | ENSMUST00000004936.6 | M23 |
| | Cybb | ENSMUST00000015484.5 | M23 |
| | Clec4n | ENSMUST00000024118.6 | M23 |
| | Cbr2 | ENSMUST00000026148.4 | M23 |
| | Mrc1 | ENSMUST00000028045.3 | M23 |
| | Fcna | ENSMUST00000028307.8 | M23 |
| | Pf4 | ENSMUST00000031320.6 | M23 |
| | Lyve1 | ENSMUST00000033050.3 | M23 |
| | F13a1 | ENSMUST00000037491.8 | M23 |
| | AI607873 | ENSMUST00000042610.9 | M23 |
| | Clec4a1 | ENSMUST00000060484.8 | M23 |
| | Ms4a7 | ENSMUST00000067532.6 | M23 |
| | LilrM4 | ENSMUST00000078778.3 | M23 |
| | Cd163 | ENSMUST00000112541.4 | M23 |
| | Cd209g | ENSMUST00000130372.1 | M23 |
| | Cd36 | ENSMUST00000170051.3 | M23 |
| | Msr1 | ENSMUST00000170091.1 | M23 |
| | Ms4a4a | ENSMUST00000188995.1 | M23 |
| 3r | Fam64a | ENSMUST00000021164.3 | M24 |
| | Pbk | ENSMUST00000022612.5 | M24 |
| | Casc5 | ENSMUST00000028802.2 | M24 |
| | Troap | ENSMUST00000039665.6 | M24 |
| | Kif2c | ENSMUST00000065896.4 | M24 |
| | Top2a | ENSMUST00000068031.7 | M24 |
| | CcnM1 | ENSMUST00000072119.10 | M24 |
| | Ube2c | ENSMUST00000088248.8 | M24 |
| | Cdk1 | ENSMUST00000119827.3 | M24 |
| | 1190002F15Rik | ENSMUST00000183867.3 | M24 |

In one further example, as shown in FIG. 9, the present disclosure provides gene-centric profiling using 20 gene expression programs in the mouse cortex. The gene-gene correlation analysis is performed on the 20 the gene expression programs using non-negative matrix factorization (NMF) algorithm. In one example, the plurality of pre-determined genes expressed in a gene expression program selected from Erp, ExcL2, ExcL3, ExcL4, ExcL5p1, ExcL5p2, ExcL5p3, ExcL6p1, ExcL6p2, Hip, IntCckVip, IntNpy, IntPv, IntSst, LrpD, LrpS, NS, Other, Sub and Syn. In another example, the plurality of pre-determined genes expressed in Erp include genes listed in Table 4 (4a). In another example, the plurality of pre-determined genes expressed in ExcL2 include genes listed in Table 4 (4b). In another example, the plurality of pre-determined genes expressed in ExcL3 include genes listed in Table 4 (4c). In another example, the plurality of pre-determined genes expressed in ExcL4 include genes listed in Table 4 (4d). In another example, the plurality of pre-determined genes expressed in ExcL5p1 include genes listed in Table 4 (4e). In another example, the plurality of pre-determined genes expressed in ExcL5p2 include genes listed in Table 4 (4f). In another example, the plurality of pre-determined genes expressed in ExcL5p3 include genes listed in Table 4 (4g). In another example, the plurality of pre-determined genes expressed in ExcL6p1 include genes listed in Table 4 (4h). In another example, the plurality of pre-determined genes expressed in ExcL6p2 include genes listed in Table 4 (4i). In another example, the plurality of pre-determined genes expressed in Hip include genes listed in Table 4 (4j). In another example, the plurality of pre-determined genes expressed in IntCckVip include genes listed in Table 4 (4k). In another example, the plurality of pre-determined genes expressed in IntNpy include genes listed in Table 4 (41). In another example, the plurality of pre-determined genes expressed in IntPv include genes listed in Table 4 (4m). In another example, the plurality of pre-determined genes expressed in IntSst include genes listed in Table 4 (4n). In another example, the plurality of pre-determined genes expressed in LrpD include genes listed in Table 4 (40). In another example, the plurality of pre-determined genes expressed in LrpS include genes listed in Table 4 (4p). In another example, the plurality of pre-determined genes expressed in NS include genes listed in Table 4 (4q). In another example, the plurality of pre-determined genes expressed in Other, which is characterized by high expression of non-coding RNA Meg3 and other genes that are associated with cerebral ischemic injury, include genes listed in Table 4 (4r). In another example, the plurality of pre-determined genes expressed in Sub include genes listed in Table 4 (4s). In another example, the plurality of pre-determined genes expressed in Syn include genes listed in Table 4 (4t).

TABLE 4
FISHnCHIPs for FIG. 9 Mouse Cortex Library

| | | | Gene |
|---|---|---|---|
| Table | | | expression |
| ID | Gene | Transcript ID | program |
| 4a | Ifrd1 | ENSMUST00000001672.7 | Erp |
| | Dnajb1 | ENSMUST00000005620.8 | Erp |
| | Gadd45b | ENSMUST00000015456.8 | Erp |
| | Per1 | ENSMUST00000021271.9 | Erp |
| | Gadd45g | ENSMUST00000021903.2 | Erp |
| | Dusp1 | ENSMUST00000025025.6 | Erp |
| | Ccnl1 | ENSMUST00000029416.9 | Erp |
| | Nr4a3 | ENSMUST00000030025.5 | Erp |
| | Fosl2 | ENSMUST00000031017.9 | Erp |
| | Ciart | ENSMUST00000036418.5 | Erp |
| | Arl4d | ENSMUST00000039388.2 | Erp |
| | Irs2 | ENSMUST00000040514.6 | Erp |
| | Fbxo33 | ENSMUST00000043204.7 | Erp |
| | Tiparp | ENSMUST00000047906.5 | Erp |
| | Npas4 | ENSMUST00000056129.7 | Erp |
| | Frmd6 | ENSMUST00000057859.7 | Erp |
| | Trib1 | ENSMUST00000067543.6 | Erp |
| | Cdc42ep3 | ENSMUST00000068958.7 | Erp |
| | Btaf1 | ENSMUST00000099494.3 | Erp |
| | Dusp14 | ENSMUST00000100705.6 | Erp |
| | Mest | ENSMUST00000163949.4 | Erp |
| | Arl5b | ENSMUST00000193883.1 | Erp |
| 4b | Lpl | ENSMUST00000015712.10 | ExcL2 |
| | Ngb | ENSMUST00000021420.9 | ExcL2 |
| | Pvrl3 | ENSMUST00000023334.10 | ExcL2 |
| | Bhlhe22 | ENSMUST00000026120.7 | ExcL2 |
| | Itm2c | ENSMUST00000027425.11 | ExcL2 |
| | Gucy1b3 | ENSMUST00000029635.9 | ExcL2 |
| | Pcdh8 | ENSMUST00000039568.6 | ExcL2 |
| | Wfs1 | ENSMUST00000043964.8 | ExcL2 |
| | Gucy1a3 | ENSMUST00000048976.7 | ExcL2 |
| | Dusp18 | ENSMUST00000055931.4 | ExcL2 |
| | Evc2 | ENSMUST00000056365.8 | ExcL2 |
| | Pcdh19 | ENSMUST00000060309.9 | ExcL2 |
| | Rgs14 | ENSMUST00000063771.9 | ExcL2 |
| | C2cd2l | ENSMUST00000065080.8 | ExcL2 |
| | Gsg1l | ENSMUST00000073935.5 | ExcL2 |
| | Otof | ENSMUST00000074171.8 | ExcL2 |
| | Syt17 | ENSMUST00000081574.4 | ExcL2 |
| | Ankrd6 | ENSMUST00000084750.3 | ExcL2 |
| | Rragd | ENSMUST00000098190.5 | ExcL2 |
| | Plk5 | ENSMUST00000105351.1 | ExcL2 |
| 4c | Hlf | ENSMUST00000004051.7 | ExcL3 |
| | Kcnab3 | ENSMUST00000018614.2 | ExcL3 |
| | Cacna2d3 | ENSMUST00000022567.8 | ExcL3 |
| | Alcam | ENSMUST00000023312.9 | ExcL3 |
| | Slc17a6 | ENSMUST00000032710.5 | ExcL3 |
| | Atp1a1 | ENSMUST00000036493.6 | ExcL3 |
| | Arl6ip5 | ENSMUST00000044681.6 | ExcL3 |
| | Ddit4l | ENSMUST00000053855.7 | ExcL3 |
| | Fam60a | ENSMUST00000054080.10 | ExcL3 |
| | Scn4b | ENSMUST00000060125.5 | ExcL3 |
| | Tbc1d30 | ENSMUST00000064107.5 | ExcL3 |
| | Cbln4 | ENSMUST00000087950.3 | ExcL3 |
| | Sytl2 | ENSMUST00000107211.3 | ExcL3 |
| | Lingo2 | ENSMUST00000108122.3 | ExcL3 |
| | Cux1 | ENSMUST00000176745.3 | ExcL3 |
| 4d | Plxnd1 | ENSMUST00000015511.10 | ExcL4 |
| | Krt12 | ENSMUST00000017741.3 | ExcL4 |
| | Igfbp5 | ENSMUST00000027377.8 | ExcL4 |
| | Cnih3 | ENSMUST00000027795.9 | ExcL4 |
| | Pamr1 | ENSMUST00000028612.7 | ExcL4 |
| | Dkkl1 | ENSMUST00000033057.7 | ExcL4 |
| | Rora | ENSMUST00000034766.9 | ExcL4 |
| | S100a10 | ENSMUST00000045756.9 | ExcL4 |
| | Fam19a2 | ENSMUST00000050756.7 | ExcL4 |
| | Tshz1 | ENSMUST00000060303.9 | ExcL4 |
| | Tmem65 | ENSMUST00000072113.5 | ExcL4 |
| | Kcnk2 | ENSMUST00000079451.8 | ExcL4 |
| | Scnn1a | ENSMUST00000081440.9 | ExcL4 |
| | Whrn | ENSMUST00000084510.3 | ExcL4 |
| | Coch | ENSMUST00000085412.5 | ExcL4 |
| | Shisa3 | ENSMUST00000087241.5 | ExcL4 |
| | Endou | ENSMUST00000100249.4 | ExcL4 |
| | Tmem145 | ENSMUST00000108409.1 | ExcL4 |
| | Ccdc136 | ENSMUST00000115275.3 | ExcL4 |
| | Spock3 | ENSMUST00000119068.3 | ExcL4 |
| | BC006965 | ENSMUST00000124028.3 | ExcL4 |
| | A830036E02Rik | ENSMUST00000128904.1 | ExcL4 |
| | Thbs2 | ENSMUST00000170872.1 | ExcL4 |
| | Nrep | ENSMUST00000171533.3 | ExcL4 |
| 4e | Col6a1 | ENSMUST00000001147.4 | ExcL5p1 |
| | Slc26a4 | ENSMUST00000001253.7 | ExcL5p1 |
| | Map2k1 | ENSMUST00000005066.8 | ExcL5p1 |
| | Galnt14 | ENSMUST00000024858.7 | ExcL5p1 |
| | Bmp3 | ENSMUST00000031278.4 | ExcL5p1 |
| | Arl6ip1 | ENSMUST00000032888.7 | ExcL5p1 |
| | Cbln1 | ENSMUST00000034076.10 | ExcL5p1 |
| | Clstn2 | ENSMUST00000035027.8 | ExcL5p1 |
| | Rasl10a | ENSMUST00000037218.1 | ExcL5p1 |
| | Igsf21 | ENSMUST00000039331.8 | ExcL5p1 |
| | Spon1 | ENSMUST00000046687.11 | ExcL5p1 |
| | Crtac1 | ENSMUST00000048630.6 | ExcL5p1 |
| | 1110032F04Rik | ENSMUST00000054551.2 | ExcL5p1 |
| | Osr1 | ENSMUST00000057021.7 | ExcL5p1 |
| | Rspo2 | ENSMUST00000063492.6 | ExcL5p1 |
| | Sema3e | ENSMUST00000073957.6 | ExcL5p1 |
| | Rxfp1 | ENSMUST00000078527.8 | ExcL5p1 |
| | Susd4 | ENSMUST00000085724.4 | ExcL5p1 |
| | Ptprt | ENSMUST00000109443.3 | ExcL5p1 |
| | Rimbp2 | ENSMUST00000111346.1 | ExcL5p1 |
| | D430019H16Rik | ENSMUST00000178224.1 | ExcL5p1 |
| 4f | Hcn1 | ENSMUST00000006991.7 | ExcL5p2 |
| | Trpc4 | ENSMUST00000029311.6 | ExcL5p2 |
| | Stac | ENSMUST00000035083.7 | ExcL5p2 |
| | Parm1 | ENSMUST00000040576.9 | ExcL5p2 |
| | Vat1l | ENSMUST00000049509.6 | ExcL5p2 |
| | Qrfpr | ENSMUST00000091227.7 | ExcL5p2 |
| | Nefh | ENSMUST00000093369.4 | ExcL5p2 |
| | Ntng1 | ENSMUST00000156177.4 | ExcL5p2 |
| | Cacna1h | ENSMUST00000159610.3 | ExcL5p2 |
| 4g | Chga | ENSMUST00000021610.5 | ExcL5p3 |
| | Slc6a7 | ENSMUST00000025520.8 | ExcL5p3 |
| | Esrrg | ENSMUST00000027906.8 | ExcL5p3 |
| | Tspan5 | ENSMUST00000029800.4 | ExcL5p3 |
| | Fras1 | ENSMUST00000036019.4 | ExcL5p3 |
| | Vstm2b | ENSMUST00000044705.10 | ExcL5p3 |
| | Hrh3 | ENSMUST00000056480.5 | ExcL5p3 |
| | Tmem91 | ENSMUST00000079439.5 | ExcL5p3 |
| | Wbscr17 | ENSMUST00000086023.7 | ExcL5p3 |
| | Gpr88 | ENSMUST00000090473.5 | ExcL5p3 |
| | Deptor | ENSMUST00000096433.5 | ExcL5p3 |
| | Ptgfrn | ENSMUST00000102694.3 | ExcL5p3 |
| | Il1rapl2 | ENSMUST00000113063.3 | ExcL5p3 |
| | Tmsb10 | ENSMUST00000114050.3 | ExcL5p3 |
| | Sstr2 | ENSMUST00000146390.2 | ExcL5p3 |
| | Fam3c | ENSMUST00000165576.3 | ExcL5p3 |
| 4h | Trh | ENSMUST00000006046.4 | ExcL6p1 |
| | Ctgf | ENSMUST00000020171.7 | ExcL6p1 |
| | Cidea | ENSMUST00000025404.8 | ExcL6p1 |
| | Pcsk5 | ENSMUST00000050715.8 | ExcL6p1 |
| | Gnal | ENSMUST00000076605.7 | ExcL6p1 |
| | Nxph4 | ENSMUST00000095266.2 | ExcL6p1 |
| | Syndig1l | ENSMUST00000095550.2 | ExcL6p1 |
| | Fam65b | ENSMUST00000110384.4 | ExcL6p1 |
| | Fam163b | ENSMUST00000151224.2 | ExcL6p1 |
| | Ly6g6e | ENSMUST00000172678.3 | ExcL6p1 |
| | Sulf1 | ENSMUST00000185780.1 | ExcL6p1 |
| 4i | Igfbp4 | ENSMUST00000017637.8 | ExcL6p2 |
| | Gadd45a | ENSMUST00000043098.6 | ExcL6p2 |
| | Ramp3 | ENSMUST00000045374.7 | ExcL6p2 |
| | Tle4 | ENSMUST00000052011.9 | ExcL6p2 |
| | Syt6 | ENSMUST00000090697.6 | ExcL6p2 |
| | Lrrtm2 | ENSMUST00000091636.3 | ExcL6p2 |
| | Garnl3 | ENSMUST00000102810.5 | ExcL6p2 |
| | Slc35f1 | ENSMUST00000105473.2 | ExcL6p2 |
| | Lpgat1 | ENSMUST00000110855.3 | ExcL6p2 |
| | Islr2 | ENSMUST00000114144.4 | ExcL6p2 |
| | Foxp2 | ENSMUST00000115477.3 | ExcL6p2 |
| | Cdh2 | ENSMUST00000115850.1 | ExcL6p2 |
| | Lmo3 | ENSMUST00000162772.3 | ExcL6p2 |
| | Islr | ENSMUST00000168864.2 | ExcL6p2 |
| | A830018L16Rik | ENSMUST00000171690.4 | ExcL6p2 |
| 4j | Doc2b | ENSMUST00000021209.7 | Hip |
| | Fibcd1 | ENSMUST00000028188.7 | Hip |
| | Cpne7 | ENSMUST00000037900.8 | Hip |
| | Pkp2 | ENSMUST00000039408.2 | Hip |
| | Gabra5 | ENSMUST00000068456.6 | Hip |
| | Iqgap2 | ENSMUST00000068603.6 | Hip |
| | Epha7 | ENSMUST00000080934.6 | Hip |
| | Grem1 | ENSMUST00000099575.3 | Hip |
| | Prkcg | ENSMUST00000100301.6 | Hip |
| | Nr3c2 | ENSMUST00000109912.3 | Hip |
| | Gpr161 | ENSMUST00000111450.2 | Hip |
| | Spink8 | ENSMUST00000118732.2 | Hip |
| | Scn3b | ENSMUST00000171835.4 | Hip |
| 4k | Asic4 | ENSMUST00000037708.9 | IntCckVip |
| | Egln3 | ENSMUST00000039516.3 | IntCckVip |
| | Cnr1 | ENSMUST00000057188.6 | IntCckVip |
| | Sp8 | ENSMUST00000063918.2 | IntCckVip |
| | Frem1 | ENSMUST00000071708.7 | IntCckVip |
| | Npy2r | ENSMUST00000098997.4 | IntCckVip |
| | Adarb2 | ENSMUST00000135574.3 | IntCckVip |
| 4l | Kit | ENSMUST00000005815.6 | IntNpy |
| | Ngf | ENSMUST00000035952.3 | IntNpy |
| | Slc35d3 | ENSMUST00000059805.4 | IntNpy |
| | Sp9 | ENSMUST00000090813.5 | IntNpy |
| | Baiap2l2 | ENSMUST00000165408.3 | IntNpy |
| | Tnnt1 | ENSMUST00000166959.3 | IntNpy |
| 4m | Akr1c18 | ENSMUST00000021635.7 | IntPv |
| | Kcnc1 | ENSMUST00000025202.6 | IntPv |
| | Vamp1 | ENSMUST00000032487.9 | IntPv |
| | Cox6a2 | ENSMUST00000033049.7 | IntPv |
| | Lrrc38 | ENSMUST00000052458.2 | IntPv |
| | Ankrd34b | ENSMUST00000061594.8 | IntPv |
| | Nog | ENSMUST00000061728.4 | IntPv |
| | Kcnc2 | ENSMUST00000092175.2 | IntPv |
| | Btbd11 | ENSMUST00000105306.2 | IntPv |
| | Tmem132c | ENSMUST00000119026.3 | IntPv |
| | Kcnmb2 | ENSMUST00000119310.3 | IntPv |
| | Ank1 | ENSMUST00000121802.4 | IntPv |
| | Ppargc1a | ENSMUST00000132734.3 | IntPv |
| | A330050F15Rik | ENSMUST00000169935.1 | IntPv |
| 4n | Ache | ENSMUST00000024099.6 | IntSst |
| | Lypd6b | ENSMUST00000028103.8 | IntSst |
| | Pdyn | ENSMUST00000028883.7 | IntSst |
| | Dlx1 | ENSMUST00000037119.3 | IntSst |
| | Grm1 | ENSMUST00000044306.8 | IntSst |
| | AF529169 | ENSMUST00000044491.8 | IntSst |
| | Elfn1 | ENSMUST00000050519.6 | IntSst |
| | Oxtr | ENSMUST00000053306.6 | IntSst |
| | Kctd8 | ENSMUST00000054095.4 | IntSst |
| | Rxfp3 | ENSMUST00000058007.6 | IntSst |
| | AI504432 | ENSMUST00000070085.5 | IntSst |
| | Rpp25 | ENSMUST00000080514.7 | IntSst |
| | Elavl2 | ENSMUST00000107120.3 | IntSst |
| | Vstm2a | ENSMUST00000109645.4 | IntSst |
| | Rbp4 | ENSMUST00000112335.2 | IntSst |
| | Lhx6 | ENSMUST00000112960.3 | IntSst |
| | Col19a1 | ENSMUST00000115244.4 | IntSst |
| | Cdh13 | ENSMUST00000117160.1 | IntSst |
| 4o | Tpbg | ENSMUST00000006559.9 | LrpD |
| | Efr3a | ENSMUST00000015146.11 | LrpD |
| | Hspa8 | ENSMUST00000015800.11 | LrpD |
| | Hspa4 | ENSMUST00000020630.7 | LrpD |
| | Tmem178 | ENSMUST00000025092.4 | LrpD |
| | Spred1 | ENSMUST00000028829.8 | LrpD |
| | Hectd2 | ENSMUST00000047247.7 | LrpD |
| | Ier5 | ENSMUST00000055322.5 | LrpD |
| | Arhgef7 | ENSMUST00000110909.4 | LrpD |
| | Cltc | ENSMUST00000124385.1 | LrpD |
| | Fmnl1 | ENSMUST00000129726.2 | LrpD |
| | Crem | ENSMUST00000139537.1 | LrpD |
| | Csnk1a1 | ENSMUST00000165123.3 | LrpD |
| | Ndfip2 | ENSMUST00000181969.3 | LrpD |
| | Pdlim1 | ENSMUST00000182432.1 | LrpD |
| 4p | Grasp | ENSMUST00000000543.4 | LrpS |
| | Fam84a | ENSMUST00000020926.6 | LrpS |
| | Cdkn1a | ENSMUST00000023829.6 | LrpS |
| | Lrrk2 | ENSMUST00000060642.6 | LrpS |
| | C1ql3 | ENSMUST00000061545.6 | LrpS |
| | Penk | ENSMUST00000070375.7 | LrpS |
| | Nptx2 | ENSMUST00000071782.6 | LrpS |
| | Tsnax | ENSMUST00000075896.6 | LrpS |
| | Nhp2l1 | ENSMUST00000080622.7 | LrpS |
| | Car12 | ENSMUST00000085420.7 | LrpS |
| | Mapk4 | ENSMUST00000091851.5 | LrpS |
| | Pak6 | ENSMUST00000099557.5 | LrpS |
| | Tpm1 | ENSMUST00000113690.3 | LrpS |
| | Sorbs2 | ENSMUST00000125295.3 | LrpS |
| | Prkg2 | ENSMUST00000161490.3 | LrpS |
| | Inhba | ENSMUST00000164993.1 | LrpS |
| | Mas1 | ENSMUST00000165020.3 | LrpS |
| | Actn1 | ENSMUST00000167327.1 | LrpS |
| 4q | Nme1 | ENSMUST00000021220.5 | NS |
| | Atp6v0c | ENSMUST00000024932.7 | NS |
| | Impact | ENSMUST00000025290.5 | NS |
| | Neurod6 | ENSMUST00000044767.8 | NS |
| | Fxyd6 | ENSMUST00000085939.6 | NS |
| | Tagln3 | ENSMUST00000096057.4 | NS |
| | Gnas | ENSMUST00000109087.3 | NS |
| | Etl4 | ENSMUST00000114604.4 | NS |
| | Cadps2 | ENSMUST00000115358.4 | NS |
| | Pld3 | ENSMUST00000117095.3 | NS |
| 4r | Sec62 | ENSMUST00000029256.8 | Other |
| | Vcp | ENSMUST00000030164.7 | Other |
| | Klf9 | ENSMUST00000036884.1 | Other |
| | Pde4a | ENSMUST00000039413.10 | Other |
| | Bzrap1 | ENSMUST00000039627.7 | Other |
| | Zbtb7a | ENSMUST00000048128.10 | Other |
| | Pisd | ENSMUST00000061895.11 | Other |
| | Klf13 | ENSMUST00000063694.8 | Other |
| | Usp2 | ENSMUST00000065461.7 | Other |
| | Snrnp70 | ENSMUST00000074575.7 | Other |
| | Rsrp1 | ENSMUST00000078084.6 | Other |
| | Nkain3 | ENSMUST00000102998.3 | Other |
| | Nfix | ENSMUST00000109764.3 | Other |
| | Pabpn1 | ENSMUST00000116476.4 | Other |
| | Adcy9 | ENSMUST00000117801.3 | Other |
| | Glg1 | ENSMUST00000169020.3 | Other |
| | Miat | ENSMUST00000183036.1 | Other |
| | Srrm2 | ENSMUST00000190686.2 | Other |
| 4s | Slc17a8 | ENSMUST00000020102.9 | Sub |
| | Fezf2 | ENSMUST00000022262.4 | Sub |
| | Cdhr1 | ENSMUST00000022337.9 | Sub |
| | Grp | ENSMUST00000025395.8 | Sub |
| | Lypd1 | ENSMUST00000027582.5 | Sub |
| | Col24a1 | ENSMUST00000029848.4 | Sub |
| | Htr2c | ENSMUST00000036303.4 | Sub |
| | Trhr | ENSMUST00000038856.8 | Sub |
| | Vwc2l | ENSMUST00000053922.7 | Sub |
| | Rnf152 | ENSMUST00000058688.6 | Sub |
| | Glra2 | ENSMUST00000058787.8 | Sub |
| | Stard5 | ENSMUST00000075418.9 | Sub |
| | St3gal1 | ENSMUST00000092640.5 | Sub |
| | Myl4 | ENSMUST00000106956.5 | Sub |
| | Tshz2 | ENSMUST00000109157.1 | Sub |
| | Sla2 | ENSMUST00000109561.3 | Sub |
| | Neto2 | ENSMUST00000109686.3 | Sub |
| | Plcxd2 | ENSMUST00000130481.1 | Sub |
| | Etv1 | ENSMUST00000159334.3 | Sub |
| | Nxph1 | ENSMUST00000160300.1 | Sub |
| 4t | Grk4 | ENSMUST00000001112.9 | Syn |
| | Mrps18c | ENSMUST00000016977.10 | Syn |
| | Taok1 | ENSMUST00000017435.6 | Syn |
| | Papolg | ENSMUST00000020513.5 | Syn |
| | Gsk3b | ENSMUST00000023507.8 | Syn |
| | Cxcr2 | ENSMUST00000027372.7 | Syn |
| | Gnb1 | ENSMUST00000030940.9 | Syn |
| | Fam81a | ENSMUST00000034749.10 | Syn |
| | Pura | ENSMUST00000051301.3 | Syn |
| | Olfr56 | ENSMUST00000056759.6 | Syn |
| | Kcnb1 | ENSMUST00000059826.8 | Syn |
| | Bicd1 | ENSMUST00000086829.6 | Syn |
| | Nol4 | ENSMUST00000092015.6 | Syn |
| | Med23 | ENSMUST00000092646.8 | Syn |
| | Prkacb | ENSMUST00000102515.5 | Syn |
| | Ncam1 | ENSMUST00000114476.3 | Syn |
| | Cadm1 | ENSMUST00000114547.3 | Syn |
| | Eid1 | ENSMUST00000164756.3 | Syn |
| | Ank2 | ENSMUST00000182078.3 | Syn |

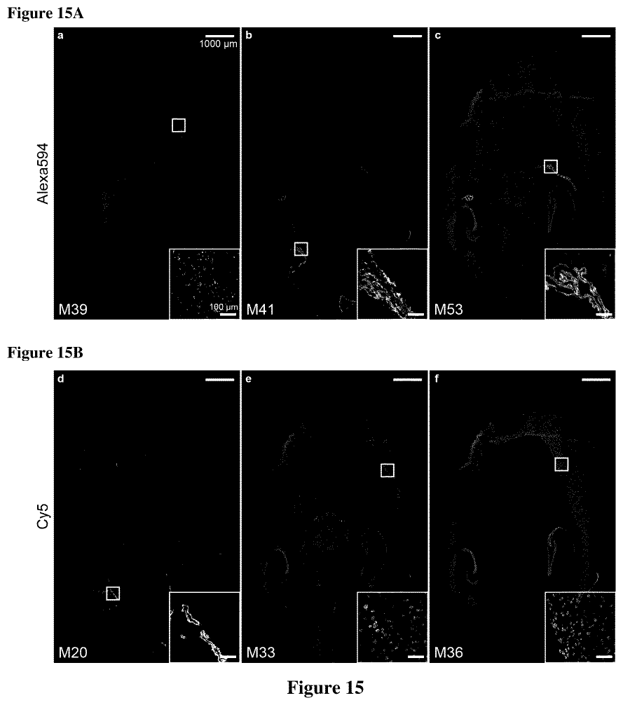

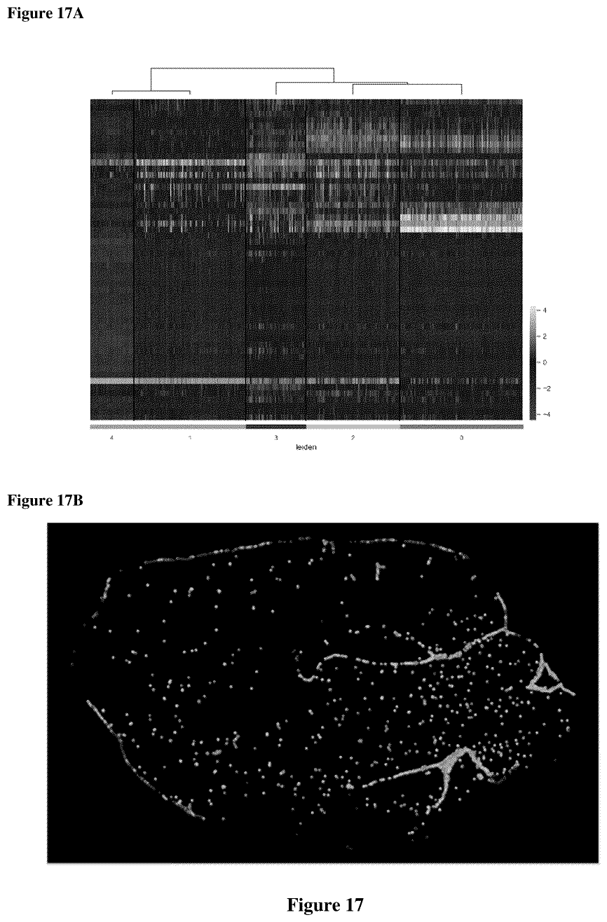

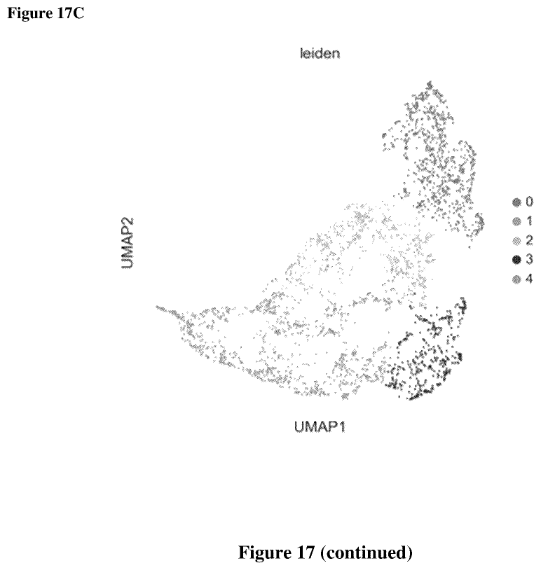

In one example, the plurality of pre-determined genes is expressed in the mouse brain as shown in FIG. 13 to FIG. 18. In one example, the plurality of pre-determined genes expressed in a gene module selected from any one of the gene modules M1 to M53.

In one example, the plurality of pre-determined genes expressed in M1 gene module include genes listed in Table 5 (5a). In another example, the plurality of pre-determined genes expressed in M2 gene module include genes listed in Table 5 (5b). In another example, the plurality of pre-determined genes expressed in M3 gene module include genes listed in Table 5 (5c). In another example, the plurality of pre-determined genes expressed in M4 gene module include genes listed in Table 5 (5d). In another example, the plurality of pre-determined genes expressed in M5 gene module include genes listed in Table 5 (5e). In another example, the plurality of pre-determined genes expressed in M6 gene module include genes listed in Table 5 (5f). In another example, the plurality of pre-determined genes expressed in M7 gene module include genes listed in Table 5 (5g). In another example, the plurality of pre-determined genes expressed in M8 gene module include genes listed in Table 5 (5h). In another example, the plurality of pre-determined genes expressed in M9 gene module include genes listed in Table 5 (5i). In another example, the plurality of pre-determined genes expressed in M10 gene module include genes listed in Table 5 (5j). In another example, the plurality of pre-determined genes expressed in M11 gene module include genes listed in Table 5 (5k). In another example, the plurality of pre-determined genes expressed in M12 gene module include genes listed in Table 5 (51). In another example, the plurality of pre-determined genes expressed in M13 gene module include genes listed in Table 5 (5m). In another example, the plurality of pre-determined genes expressed in M14 gene module include genes listed in Table 5 (5n). In another example, the plurality of pre-determined genes expressed in M15 gene module include genes listed in Table 5 (50). In another example, the plurality of pre-determined genes expressed in M16 gene module include genes listed in Table 5 (5p). In another example, the plurality of pre-determined genes expressed in M17 gene module include genes listed in Table 5 (5q). In another example, the plurality of pre-determined genes expressed in M18 gene module include genes listed in Table 5 (5r). In another example, the plurality of pre-determined genes expressed in M19 gene module include genes listed in Table 5 (5s). In another example, the plurality of pre-determined genes expressed in M20 gene module include genes listed in Table 5 (5t). In another example, the plurality of pre-determined genes expressed in M21 gene module include genes listed in Table 5 (5u). In another example, the plurality of pre-determined genes expressed in M22 gene module include genes listed in Table 5 (5v). In another example, the plurality of pre-determined genes expressed in M23 gene module include genes listed in Table 5 (5w). In another example, the plurality of pre-determined genes expressed in M24 gene module include genes listed in Table 5 (5×). In another example, the plurality of pre-determined genes expressed in M25 gene module include genes listed in Table 5 (5y). In another example, the plurality of pre-determined genes expressed in M26 gene module include genes listed in Table 5 (5z). In another example, the plurality of pre-determined genes expressed in M27 gene module include genes listed in Table 5 (5aa). In another example, the plurality of pre-determined genes expressed in M28 gene module include genes listed in Table 5 (5ab). In another example, the plurality of pre-determined genes expressed in M29 gene module include genes listed in Table 5 (5ac). In another example, the plurality of pre-determined genes expressed in M30 gene module include genes listed in Table 5 (5ad). In another example, the plurality of pre-determined genes expressed in M31 gene module include genes listed in Table 5 (5ae). In another example, the plurality of pre-determined genes expressed in M32 gene module include genes listed in Table 5 (5af). In another example, the plurality of pre-determined genes expressed in M33 gene module include genes listed in Table 5 (5ag). In another example, the plurality of pre-determined genes expressed in M34 gene module include genes listed in Table 5 (5ah). In another example, the plurality of pre-determined genes expressed in M35 gene module include genes listed in Table 5 (5ai). In another example, the plurality of pre-determined genes expressed in M36 gene module include genes listed in Table 5 (5aj). In another example, the plurality of pre-determined genes expressed in M37 gene module include genes listed in Table 5 (5ak). In another example, the plurality of pre-determined genes expressed in M38 gene module include genes listed in Table 5 (5al). In another example, the plurality of pre-determined genes expressed in M39 gene module include genes listed in Table 5 (5 am). In another example, the plurality of pre-determined genes expressed in M40 gene module include genes listed in Table 5 (5an). In another example, the plurality of pre-determined genes expressed in M41 gene module include genes listed in Table 5 (5ao). In another example, the plurality of pre-determined genes expressed in M42 gene module include genes listed in Table 5 (5ap). In another example, the plurality of pre-determined genes expressed in M43 gene module include genes listed in Table 5 (5aq). In another example, the plurality of pre-determined genes expressed in M44 gene module include genes listed in Table 5 (5ar). In another example, the plurality of pre-determined genes expressed in M45 gene module include genes listed in Table 5 (5as). In another example, the plurality of pre-determined genes expressed in M46 gene module include genes listed in Table 5 (5at). In another example, the plurality of pre-determined genes expressed in M47 gene module include genes listed in Table 5 (5au). In another example, the plurality of pre-determined genes expressed in M48 gene module include genes listed in Table 5 (5av). In another example, the plurality of pre-determined genes expressed in M49 gene module include genes listed in Table 5 (5aw). In another example, the plurality of pre-determined genes expressed in M50 gene module include genes listed in Table 5 (5ax). In another example, the plurality of pre-determined genes expressed in M51 gene module include genes listed in Table 5 (5ay). In another example, the plurality of pre-determined genes expressed in M52 gene module include genes listed in Table 5 (5az). In another example, the plurality of pre-determined genes expressed in M53 gene module include genes listed in Table 5 (5ba).

TABLE 5
FISHnCHIPs for FIG. 13 Mouse Brain Library

| Table | | | Gene |
|---|---|---|---|
| ID | Gene | Transcription ID | module |
| 5a | Zwint | ENSMUSG00000019923.9 | M1 |
| | Nsg2 | ENSMUSG00000020297.6 | M1 |
| | Plk2 | ENSMUSG00000021701.7 | M1 |
| | Syt4 | ENSMUSG00000024261.5 | M1 |
| | Uchl1 | ENSMUSG00000029223.9 | M1 |
| | Aldoa | ENSMUSG00000030695.9 | M1 |
| | Cck | ENSMUSG00000032532.6 | M1 |
| | Neurod6 | ENSMUSG00000037984.8 | M1 |
| | Junb | ENSMUSG00000052837.5 | M1 |
| | Egr4 | ENSMUSG00000071341.3 | M1 |
| | Snca | ENSMUSG00000025889.9 | M1 |
| | Nell2 | ENSMUSG00000022454.12 | M1 |
| | Syn2 | ENSMUSG00000009394.9 | M1 |
| | Nptxr | ENSMUSG00000022421.14 | M1 |
| 5b | Gpr123 | ENSMUSG00000025475.13 | M2 |
| | Galnt9 | ENSMUSG00000033316.10 | M2 |
| | Map1b | ENSMUSG00000052727.5 | M2 |
| | Gda | ENSMUSG00000058624.8 | M2 |
| | Epha5 | ENSMUSG00000029245.12 | M2 |
| | Meg3 | ENSMUSG00000021268.13 | M2 |
| | Nos1ap | ENSMUSG00000038473.10 | M2 |
| | Anks1b | ENSMUSG00000058589.10 | M2 |
| 5c | Cd68 | ENSMUSG00000018774.9 | M3 |
| | Ly86 | ENSMUSG00000021423.5 | M3 |
| | Rnase4 | ENSMUSG00000021876.10 | M3 |
| | Hpgds | ENSMUSG00000029919.4 | M3 |
| | Rgs10 | ENSMUSG00000030844.7 | M3 |
| | Stab1 | ENSMUSG00000042286.9 | M3 |
| | Itgam | ENSMUSG00000030786.14 | M3 |
| | Fcgr2b | ENSMUSG00000026656.11 | M3 |
| | Emr1 | ENSMUSG00000004730.10 | M3 |
| | Fcrls | ENSMUSG00000015852.9 | M3 |
| | Gpr34 | ENSMUSG00000040229.7 | M3 |
| | Ltc4s | ENSMUSG00000020377.10 | M3 |
| | Fcgr3 | ENSMUSG00000059498.9 | M3 |
| | P2ry12 | ENSMUSG00000036353.9 | M3 |
| 5d | Chd5 | ENSMUSG00000005045.12 | M4 |
| | Ptk2b | ENSMUSG00000059456.9 | M4 |
| | Nefl | ENSMUSG00000022055.7 | M4 |
| | Mal2 | ENSMUSG00000024479.2 | M4 |
| | Ina | ENSMUSG00000034336.3 | M4 |
| | Sorl1 | ENSMUSG00000049313.8 | M4 |
| | Ldb2 | ENSMUSG00000039706.7 | M4 |
| | Gria3 | ENSMUSG00000001986.12 | M4 |
| | Sv2b | ENSMUSG00000053025.9 | M4 |
| | Slc4a10 | ENSMUSG00000026904.13 | M4 |
| | Elavl4 | ENSMUSG00000028546.13 | M4 |
| | Egr1 | ENSMUSG00000038418.7 | M4 |
| | Pak3 | ENSMUSG00000031284.12 | M4 |
| 5e | Kit | ENSMUSG00000005672.8 | M5 |
| | Npy | ENSMUSG00000029819.6 | M5 |
| | Dner | ENSMUSG00000036766.8 | M5 |
| | Dlx6os1 | ENSMUSG00000090063.4 | M5 |
| | Arl4c | ENSMUSG00000049866.8 | M5 |
| 5f | Csf1r | ENSMUSG00000024621.11 | M6 |
| | Ctsc | ENSMUSG00000030560.12 | M6 |
| | Tyrobp | ENSMUSG00000030579.9 | M6 |
| | C1qa | ENSMUSG00000036887.5 | M6 |
| | C1qc | ENSMUSG00000036896.5 | M6 |
| | C1qb | ENSMUSG00000036905.8 | M6 |
| | Fcer1g | ENSMUSG00000058715.7 | M6 |
| | Lyz2 | ENSMUSG00000069516.7 | M6 |
| | Ctss | ENSMUSG00000038642.6 | M6 |
| | Laptm5 | ENSMUSG00000028581.13 | M6 |
| | Aif1 | ENSMUSG00000024397.10 | M6 |
| | Ptpn18 | ENSMUSG00000026126.11 | M6 |
| 5g | Ly6h | ENSMUSG00000022577.12 | M7 |
| | Cplx2 | ENSMUSG00000025867.8 | M7 |
| | Ppfia2 | ENSMUSG00000053825.10 | M7 |
| | Ncdn | ENSMUSG00000028833.9 | M7 |
| | Dgkb | ENSMUSG00000036095.10 | M7 |
| | Prkca | ENSMUSG00000050965.10 | M7 |
| | Ddn | ENSMUSG00000059213.6 | M7 |
| | Gnal | ENSMUSG00000024524.12 | M7 |
| | Ociad2 | ENSMUSG00000029153.8 | M7 |
| | Erc2 | ENSMUSG00000040640.9 | M7 |
| | Olfm1 | ENSMUSG00000026833.14 | M7 |
| | Camk2b | ENSMUSG00000057897.10 | M7 |
| | Ildr2 | ENSMUSG00000040612.9 | M7 |
| | 1700020I14Rik | ENSMUSG00000085438.1 | M7 |
| | 2010300C02Rik | ENSMUSG00000026090.12 | M7 |
| | Arpp21 | ENSMUSG00000032503.13 | M7 |
| | Wipf3 | ENSMUSG00000086040.4 | M7 |
| | Cacna1e | ENSMUSG00000004110.10 | M7 |
| 5h | Pdgfra | ENSMUSG00000029231.11 | M8 |
| | Scrg1 | ENSMUSG00000031610.3 | M8 |
| | Olig2 | ENSMUSG00000039830.8 | M8 |
| | Sox10 | ENSMUSG00000033006.9 | M8 |
| | Neu4 | ENSMUSG00000034000.11 | M8 |
| | Olig1 | ENSMUSG00000046160.6 | M8 |
| | C1ql1 | ENSMUSG00000045532.5 | M8 |
| | Gpr17 | ENSMUSG00000052229.5 | M8 |
| | Vcan | ENSMUSG00000021614.12 | M8 |
| | Lhfpl4 | ENSMUSG00000042873.10 | M8 |
| 5i | Esam | ENSMUSG00000001946.9 | M9 |
| | Nfkbia | ENSMUSG00000021025.7 | M9 |
| | Ctla2a | ENSMUSG00000044258.9 | M9 |
| | Slc2a1 | ENSMUSG00000028645.6 | M9 |
| | Itm2a | ENSMUSG00000031239.5 | M9 |
| | Cldn5 | ENSMUSG00000041378.1 | M9 |
| | Abcb1a | ENSMUSG00000040584.8 | M9 |
| | Tmem252 | ENSMUSG00000048572.4 | M9 |
| | Pltp | ENSMUSG00000017754.9 | M9 |
| | Clec14a | ENSMUSG00000045930.2 | M9 |
| | Ly6c1 | ENSMUSG00000079018.6 | M9 |
| | Ptprb | ENSMUSG00000020154.9 | M9 |
| | Ahnak | ENSMUSG00000069833.8 | M9 |
| | Cd93 | ENSMUSG00000027435.8 | M9 |
| | Pecam1 | ENSMUSG00000020717.15 | M9 |
| | Tgm2 | ENSMUSG00000037820.11 | M9 |
| | Lsr | ENSMUSG00000001247.12 | M9 |
| | Atox1 | ENSMUSG00000018585.9 | M9 |
| | Pcp4l1 | ENSMUSG00000038370.6 | M9 |
| | Gpr116 | ENSMUSG00000056492.5 | M9 |
| | Ramp2 | ENSMUSG00000001240.9 | M9 |
| | Vwf | ENSMUSG00000001930.13 | M9 |
| | Slco1a4 | ENSMUSG00000030237.10 | M9 |
| | Iqgap1 | ENSMUSG00000030536.9 | M9 |
| | Ly6a | ENSMUSG00000075602.6 | M9 |
| | Lybe | ENSMUSG00000022587.10 | M9 |
| 5j | Dct | ENSMUSG00000022129.3 | M10 |
| | Lims2 | ENSMUSG00000024395.7 | M10 |
| | Cd9 | ENSMUSG00000030342.8 | M10 |
| | Enpp6 | ENSMUSG00000038173.10 | M10 |
| | Sirt2 | ENSMUSG00000015149.9 | M10 |
| | Bmp4 | ENSMUSG00000021835.10 | M10 |
| | Bfsp2 | ENSMUSG00000032556.10 | M10 |
| | Bcas1 | ENSMUSG00000013523.9 | M10 |
| 5k | Tgfbr1 | ENSMUSG00000007613.11 | M11 |
| | Apbb1ip | ENSMUSG00000026786.10 | M11 |
| | Ctsz | ENSMUSG00000016256.10 | M11 |
| | Serinc3 | ENSMUSG00000017707.9 | M11 |
| | Ifngr1 | ENSMUSG00000020009.8 | M11 |
| | 4632428N05Rik | ENSMUSG00000020101.10 | M11 |
| | Lgmn | ENSMUSG00000021190.10 | M11 |
| | Hexb | ENSMUSG00000021665.7 | M11 |
| | Trem2 | ENSMUSG00000023992.10 | M11 |
| | Olfml3 | ENSMUSG00000027848.11 | M11 |
| | Il6ra | ENSMUSG00000027947.7 | M11 |
| | P2ry13 | ENSMUSG00000036362.1 | M11 |
| | Zfhx3 | ENSMUSG00000038872.9 | M11 |
| | Grn | ENSMUSG00000034708.7 | M11 |
| | Ptgs1 | ENSMUSG00000047250.9 | M11 |
| | Tmem119 | ENSMUSG00000054675.5 | M11 |
| | Mpeg1 | ENSMUSG00000046805.9 | M11 |
| | Selplg | ENSMUSG00000048163.9 | M11 |
| | Itgb5 | ENSMUSG00000022817.10 | M11 |
| | Ctsd | ENSMUSG00000007891.11 | M11 |
| | Unc93b1 | ENSMUSG00000036908.12 | M11 |
| | Siglech | ENSMUSG00000051504.14 | M11 |
| | Cx3cr1 | ENSMUSG00000052336.6 | M11 |
| 5l | Gabra2 | ENSMUSG00000000560.5 | M12 |
| | Enc1 | ENSMUSG00000041773.7 | M12 |
| | Nfib | ENSMUSG00000008575.13 | M12 |
| | Epha7 | ENSMUSG00000028289.8 | M12 |
| | Prkce | ENSMUSG00000045038.10 | M12 |
| | Celf2 | ENSMUSG00000002107.14 | M12 |
| | Kcnf1 | ENSMUSG00000051726.6 | M12 |
| 5m | Rap1gds1 | ENSMUSG00000028149.8 | M13 |
| | Dkk3 | ENSMUSG00000030772.5 | M13 |
| | Bcl11b | ENSMUSG00000048251.11 | M13 |
| | Vsnl1 | ENSMUSG00000054459.6 | M13 |
| | Rph3a | ENSMUSG00000029608.7 | M13 |
| | Garnl3 | ENSMUSG00000038860.11 | M13 |
| | Ccl27a | ENSMUSG00000073888.8 | M13 |
| | Lpgat1 | ENSMUSG00000026623.12 | M13 |
| | Adora1 | ENSMUSG00000042429.8 | M13 |
| 5n | Cnn1 | ENSMUSG00000001349.4 | M14 |
| | Ntn4 | ENSMUSG00000020019.4 | M14 |
| | Sncg | ENSMUSG00000023064.4 | M14 |
| | Map3k7cl | ENSMUSG00000025610.7 | M14 |
| | Des | ENSMUSG00000026208.9 | M14 |
| | Vim | ENSMUSG00000026728.5 | M14 |
| | Tagln | ENSMUSG00000032085.4 | M14 |
| | Wtip | ENSMUSG00000036459.11 | M14 |
| | Pde3a | ENSMUSG00000041741.9 | M14 |
| | Pln | ENSMUSG00000038583.8 | M14 |
| | Fbxl22 | ENSMUSG00000050503.8 | M14 |
| | Tinagl1 | ENSMUSG00000028776.10 | M14 |
| | Palld | ENSMUSG00000058056.11 | M14 |
| | Zak | ENSMUSG00000004085.10 | M14 |
| | Flna | ENSMUSG00000031328.11 | M14 |
| | Myl6 | ENSMUSG00000090841.1 | M14 |
| 5o | Btbd17 | ENSMUSG00000000202.5 | M15 |
| | Sox9 | ENSMUSG00000000567.5 | M15 |
| | Slc1a3 | ENSMUSG00000005360.10 | M15 |
| | Htra1 | ENSMUSG00000006205.9 | M15 |
| | Cxcl14 | ENSMUSG00000021508.10 | M15 |
| | Pla2g7 | ENSMUSG00000023913.13 | M15 |
| | Gpr37l1 | ENSMUSG00000026424.8 | M15 |
| | F3 | ENSMUSG00000028128.9 | M15 |
| | Msmo1 | ENSMUSG00000031604.6 | M15 |
| | Scg3 | ENSMUSG00000032181.6 | M15 |
| | Acsbg1 | ENSMUSG00000032281.7 | M15 |
| | Fam107a | ENSMUSG00000021750.11 | M15 |
| | Gjb6 | ENSMUSG00000040055.8 | M15 |
| | Atp1b2 | ENSMUSG00000041329.9 | M15 |
| | Hes5 | ENSMUSG00000048001.7 | M15 |
| | Hepacam | ENSMUSG00000046240.7 | M15 |
| | S1pr1 | ENSMUSG00000045092.7 | M15 |
| | Ppap2b | ENSMUSG00000028517.8 | M15 |
| | Fgfr3 | ENSMUSG00000054252.13 | M15 |
| | Gja1 | ENSMUSG00000050953.9 | M15 |
| | Glul | ENSMUSG00000026473.11 | M15 |
| | Sox2 | ENSMUSG00000074637.6 | M15 |
| | Fjx1 | ENSMUSG00000075012.4 | M15 |
| | Cldn10 | ENSMUSG00000022132.11 | M15 |
| | Mfge8 | ENSMUSG00000030605.11 | M15 |
| | Tril | ENSMUSG00000043496.6 | M15 |
| | Apoe | ENSMUSG00000002985.11 | M15 |
| 5p | Gltp | ENSMUSG00000011884.9 | M16 |
| | Cldn11 | ENSMUSG00000037625.7 | M16 |
| | Rnf122 | ENSMUSG00000039328.9 | M16 |
| | Ugt8a | ENSMUSG00000032854.8 | M16 |
| | Gjc3 | ENSMUSG00000056966.6 | M16 |
| | Slc44a1 | ENSMUSG00000028412.13 | M16 |
| | Cnp | ENSMUSG00000006782.12 | M16 |
| | Gm15440 | ENSMUSG00000051107.4 | M16 |
| | Adamts4 | ENSMUSG00000006403.8 | M16 |
| | Mbp | ENSMUSG00000041607.12 | M16 |
| | Tmem163 | ENSMUSG00000026347.9 | M16 |
| 5q | Igf2 | ENSMUSG00000048583.12 | M17 |
| | Col1a1 | ENSMUSG00000001506.10 | M17 |
| | Slc22a6 | ENSMUSG00000024650.4 | M17 |
| | Col1a2 | ENSMUSG00000029661.12 | M17 |
| | Pcolce | ENSMUSG00000029718.10 | M17 |
| | Bgn | ENSMUSG00000031375.13 | M17 |
| | Colec12 | ENSMUSG00000036103.8 | M17 |
| | Slc6a20a | ENSMUSG00000036814.8 | M17 |
| | Fmod | ENSMUSG00000041559.7 | M17 |
| | Slc6a13 | ENSMUSG00000030108.10 | M17 |
| | Zic1 | ENSMUSG00000032368.10 | M17 |
| | Dcn | ENSMUSG00000019929.11 | M17 |
| 5r | Rab3b | ENSMUSG00000003411.6 | M18 |
| | Ndn | ENSMUSG00000033585.4 | M18 |
| | Resp18 | ENSMUSG00000033061.11 | M18 |
| | Peg3 | ENSMUSG00000002265.11 | M18 |
| | Nap1l5 | ENSMUSG00000055430.3 | M18 |
| | Tmem130 | ENSMUSG00000043388.7 | M18 |
| | Ahi1 | ENSMUSG00000019986.12 | M18 |
| 5s | Ccl24 | ENSMUSG00000004814.7 | M19 |
| | Cbr2 | ENSMUSG00000025150.6 | M19 |
| | Mrc1 | ENSMUSG00000026712.3 | M19 |
| | Pf4 | ENSMUSG00000029373.6 | M19 |
| | Lyve1 | ENSMUSG00000030787.3 | M19 |
| | Hpgd | ENSMUSG00000031613.8 | M19 |
| | F13a1 | ENSMUSG00000039109.11 | M19 |
| | Ms4a7 | ENSMUSG00000024672.7 | M19 |
| | Maf | ENSMUSG00000055435.6 | M19 |
| | Txnip | ENSMUSG00000038393.10 | M19 |
| | Fyb | ENSMUSG00000022148.11 | M19 |
| | Ms4a6c | ENSMUSG00000079419.4 | M19 |
| | Cd36 | ENSMUSG00000002944.11 | M19 |
| 5t | Higd1b | ENSMUSG00000020928.10 | M20 |
| | Aspn | ENSMUSG00000021388.9 | M20 |
| | Mylk | ENSMUSG00000022836.10 | M20 |
| | Casq2 | ENSMUSG00000027861.9 | M20 |
| | Ndufa4l2 | ENSMUSG00000040280.9 | M20 |
| | Phldb2 | ENSMUSG00000033149.12 | M20 |
| | Acta2 | ENSMUSG00000035783.8 | M20 |
| | Mustn1 | ENSMUSG00000042485.6 | M20 |
| | Rbpms | ENSMUSG00000031586.12 | M20 |
| | Lmod1 | ENSMUSG00000048096.7 | M20 |
| | Ptrf | ENSMUSG00000004044.9 | M20 |
| | Gjc1 | ENSMUSG00000034520.10 | M20 |
| | Ebf1 | ENSMUSG00000057098.10 | M20 |
| | Myh11 | ENSMUSG00000018830.8 | M20 |
| | Tagln2 | ENSMUSG00000026547.11 | M20 |
| 5u | Id3 | ENSMUSG00000007872.3 | M21 |
| | Cd2ap | ENSMUSG00000061665.6 | M21 |
| | Edn3 | ENSMUSG00000027524.5 | M21 |
| | Cdkn1c | ENSMUSG00000037664.8 | M21 |
| | Sepp1 | ENSMUSG00000064373.7 | M21 |
| | Kitl | ENSMUSG00000019966.13 | M21 |
| | Fxyd5 | ENSMUSG00000009687.10 | M21 |
| 5v | Slc9a3r2 | ENSMUSG00000002504.10 | M22 |
| | Tpm4 | ENSMUSG00000031799.9 | M22 |
| | Tsc22d1 | ENSMUSG00000022010.15 | M22 |
| | Slco1c1 | ENSMUSG00000030235.13 | M22 |
| | 9430020K01Rik | ENSMUSG00000033960.5 | M22 |
| | Id1 | ENSMUSG00000042745.9 | M22 |
| | Foxq1 | ENSMUSG00000038415.9 | M22 |
| | Cxcl12 | ENSMUSG00000061353.7 | M22 |
| 5w | Cp | ENSMUSG00000003617.12 | M23 |
| | Cnn2 | ENSMUSG00000004665.6 | M23 |
| | Crip1 | ENSMUSG00000006360.7 | M23 |
| | Rarres2 | ENSMUSG00000009281.4 | M23 |
| | Vtn | ENSMUSG00000017344.4 | M23 |
| | Sparc | ENSMUSG00000018593.8 | M23 |
| | Ifitm3 | ENSMUSG00000025492.6 | M23 |
| | Rgs5 | ENSMUSG00000026678.6 | M23 |
| | S100a11 | ENSMUSG00000027907.4 | M23 |
| | Igfbp7 | ENSMUSG00000036256.9 | M23 |
| | Ifitm2 | ENSMUSG00000060591.8 | M23 |
| | Myl9 | ENSMUSG00000067818.6 | M23 |
| | Lgals1 | ENSMUSG00000068220.5 | M23 |
| | Itgb1 | ENSMUSG00000025809.11 | M23 |
| | Tpm2 | ENSMUSG00000028464.12 | M23 |
| | Cald1 | ENSMUSG00000029761.12 | M23 |
| | Filip1l | ENSMUSG00000043336.10 | M23 |
| 5x | Cers2 | ENSMUSG00000015714.7 | M24 |
| | Bin1 | ENSMUSG00000024381.11 | M24 |
| | Enpp2 | ENSMUSG00000022425.11 | M24 |
| | H2afj | ENSMUSG00000060032.5 | M24 |
| | Nkx6-2 | ENSMUSG00000041309.13 | M24 |
| | Ptgds | ENSMUSG00000015090.9 | M24 |
| | Josd2 | ENSMUSG00000038695.8 | M24 |
| | Desi1 | ENSMUSG00000022472.12 | M24 |
| | 2810468N07Rik | ENSMUSG00000091475.2 | M24 |
| | Gstp1 | ENSMUSG00000060803.5 | M24 |
| 5y | Chga | ENSMUSG00000021194.5 | M25 |
| | Pak1 | ENSMUSG00000030774.9 | M25 |
| | Efhd2 | ENSMUSG00000040659.3 | M25 |
| | 2900011O08Rik | ENSMUSG00000044117.8 | M25 |
| | Usp46 | ENSMUSG00000054814.10 | M25 |
| | Gabra5 | ENSMUSG00000055078.6 | M25 |
| | Fxyd7 | ENSMUSG00000036578.6 | M25 |
| | Fut9 | ENSMUSG00000055373.8 | M25 |
| | 3110035E14Rik | ENSMUSG00000067879.3 | M25 |
| 5z | Taldo1 | ENSMUSG00000025503.4 | M26 |
| | Tspan2 | ENSMUSG00000027858.9 | M26 |
| | Pllp | ENSMUSG00000031775.4 | M26 |
| | Tmeff2 | ENSMUSG00000026109.10 | M26 |
| | Gamt | ENSMUSG00000020150.9 | M26 |
| | Gjc2 | ENSMUSG00000043448.9 | M26 |
| | Plp1 | ENSMUSG00000031425.11 | M26 |
| | Slc12a2 | ENSMUSG00000024597.10 | M26 |
| | Mobp | ENSMUSG00000032517.11 | M26 |
| | Sh3gl3 | ENSMUSG00000030638.9 | M26 |
| 5aa | Eno2 | ENSMUSG00000004267.12 | M27 |
| | Fam131a | ENSMUSG00000050821.9 | M27 |
| | Tubb3 | ENSMUSG00000062380.3 | M27 |
| | Sez6 | ENSMUSG00000000632.9 | M27 |
| | Prkcg | ENSMUSG00000078816.5 | M27 |
| | Cntn1 | ENSMUSG00000055022.10 | M27 |
| 5ab | Lynx1 | ENSMUSG00000022594.10 | M28 |
| | St8sia3 | ENSMUSG00000056812.9 | M28 |
| | Kcna2 | ENSMUSG00000040724.1 | M28 |
| | Vgf | ENSMUSG00000037428.10 | M28 |
| | Nceh1 | ENSMUSG00000027698.10 | M28 |
| | Kcna1 | ENSMUSG00000047976.3 | M28 |
| | Scn1b | ENSMUSG00000019194.10 | M28 |
| 5ac | Adcy1 | ENSMUSG00000020431.5 | M29 |
| | Tmem132a | ENSMUSG00000024736.10 | M29 |
| | Slc17a7 | ENSMUSG00000070570.4 | M29 |
| | Trbc2 | ENSMUSG00000076498.2 | M29 |
| | Atp6v1a | ENSMUSG00000052459.9 | M29 |
| 5ad | Phyhip | ENSMUSG00000003469.5 | M30 |
| | Ryr2 | ENSMUSG00000021313.11 | M30 |
| | R3hdm1 | ENSMUSG00000056211.9 | M30 |
| | Atp2b4 | ENSMUSG00000026463.13 | M30 |
| | Cdk5r1 | ENSMUSG00000048895.13 | M30 |
| | Frrs1l | ENSMUSG00000045589.7 | M30 |
| | Grin2b | ENSMUSG00000030209.10 | M30 |
| | Lamp5 | ENSMUSG00000027270.10 | M30 |
| | Lppr4 | ENSMUSG00000044667.8 | M30 |
| | C1ql3 | ENSMUSG00000049630.6 | M30 |
| | Nrgn | ENSMUSG00000053310.7 | M30 |
| | Chst1 | ENSMUSG00000027221.5 | M30 |
| | Celf1 | ENSMUSG00000005506.12 | M30 |
| | Ppp3ca | ENSMUSG00000028161.13 | M30 |
| | Ntm | ENSMUSG00000059974.6 | M30 |
| | Baiap2 | ENSMUSG00000025372.12 | M30 |
| | D430041D05Rik | ENSMUSG00000068373.10 | M30 |
| | Scn2a1 | ENSMUSG00000075318.8 | M30 |
| | Cacna2d1 | ENSMUSG00000040118.11 | M30 |
| | Camk2a | ENSMUSG00000024617.12 | M30 |
| | Rbfox3 | ENSMUSG00000025576.13 | M30 |
| | Ajap1 | ENSMUSG00000039546.9 | M30 |
| | Tenm4 | ENSMUSG00000048078.12 | M30 |
| | Fbxw7 | ENSMUSG00000028086.10 | M30 |
| | Cbx6 | ENSMUSG00000089715.7 | M30 |
| | Camkk2 | ENSMUSG00000029471.9 | M30 |
| | Cnksr2 | ENSMUSG00000025658.12 | M30 |
| | Ttc3 | ENSMUSG00000040785.13 | M30 |
| | Auts2 | ENSMUSG00000029673.13 | M30 |
| | Arhgap32 | ENSMUSG00000041444.10 | M30 |
| | Smarca2 | ENSMUSG00000024921.12 | M30 |
| | Map9 | ENSMUSG00000033900.9 | M30 |
| 5ae | Chgb | ENSMUSG00000027350.8 | M31 |
| | Pde1a | ENSMUSG00000059173.15 | M31 |
| | Caln1 | ENSMUSG00000060371.8 | M31 |
| | Kalrn | ENSMUSG00000061751.11 | M31 |
| | Synj1 | ENSMUSG00000022973.13 | M31 |
| 5af | Nid1 | ENSMUSG00000005397.7 | M32 |
| | Cox4i2 | ENSMUSG00000009876.9 | M32 |
| | Gpx8 | ENSMUSG00000021760.3 | M32 |
| | Pdgfrb | ENSMUSG00000024620.7 | M32 |
| | Enpep | ENSMUSG00000028024.10 | M32 |
| | Gng11 | ENSMUSG00000032766.8 | M32 |
| | Kcnj8 | ENSMUSG00000030247.7 | M32 |
| | Ecm2 | ENSMUSG00000043631.7 | M32 |
| | Itga1 | ENSMUSG00000042284.9 | M32 |
| | Gper1 | ENSMUSG00000053647.4 | M32 |
| | Cd248 | ENSMUSG00000056481.6 | M32 |
| | Ace2 | ENSMUSG00000015405.11 | M32 |
| | Atp13a5 | ENSMUSG00000048939.9 | M32 |
| | P2ry14 | ENSMUSG00000036381.9 | M32 |
| | Abcc9 | ENSMUSG00000030249.11 | M32 |
| | Sod3 | ENSMUSG00000072941.4 | M32 |
| | Ifitm1 | ENSMUSG00000025491.10 | M32 |
| | Eva1b | ENSMUSG00000050212.4 | M32 |
| | Art3 | ENSMUSG00000034842.12 | M32 |
| 5ag | Pvalb | ENSMUSG00000005716.12 | M33 |
| | Cacng2 | ENSMUSG00000019146.2 | M33 |
| | Lgi2 | ENSMUSG00000039252.7 | M33 |
| | Scn1a | ENSMUSG00000064329.9 | M33 |
| | Atp1a3 | ENSMUSG00000040907.11 | M33 |
| | Slc38a1 | ENSMUSG00000023169.10 | M33 |
| | Nefh | ENSMUSG00000020396.8 | M33 |
| 5ah | Eng | ENSMUSG00000026814.12 | M34 |
| | Edn1 | ENSMUSG00000021367.7 | M34 |
| | Epas1 | ENSMUSG00000024140.9 | M34 |
| | Podxl | ENSMUSG00000025608.9 | M34 |
| | Tm4sf1 | ENSMUSG00000027800.10 | M34 |
| | Flt1 | ENSMUSG00000029648.9 | M34 |
| | Gkn3 | ENSMUSG00000030048.4 | M34 |
| | Ctsh | ENSMUSG00000032359.10 | M34 |
| | Tspo | ENSMUSG00000041736.6 | M34 |
| | Uaca | ENSMUSG00000034485.9 | M34 |
| | Sdpr | ENSMUSG00000045954.7 | M34 |
| | Klf2 | ENSMUSG00000055148.7 | M34 |
| | Cgnl1 | ENSMUSG00000032232.10 | M34 |
| | Utrn | ENSMUSG00000019820.9 | M34 |
| | Sema3g | ENSMUSG00000021904.5 | M34 |
| | Tek | ENSMUSG00000006386.11 | M34 |
| | Mmrn2 | ENSMUSG00000041445.8 | M34 |
| | Bmx | ENSMUSG00000031377.7 | M34 |
| | Ltbp4 | ENSMUSG00000040488.12 | M34 |
| | Heg1 | ENSMUSG00000075254.7 | M34 |
| | Fn1 | ENSMUSG00000026193.11 | M34 |
| 5ai | Crym | ENSMUSG00000030905.5 | M35 |
| | B3galt2 | ENSMUSG00000033849.3 | M35 |
| | Bex2 | ENSMUSG00000042750.7 | M35 |
| | Hpcal4 | ENSMUSG00000046093.5 | M35 |
| | Mllt11 | ENSMUSG00000053192.5 | M35 |
| | Atp6v1g2 | ENSMUSG00000024403.12 | M35 |
| | Hs3st2 | ENSMUSG00000046321.7 | M35 |
| | Hs6st2 | ENSMUSG00000062184.7 | M35 |
| | Lrrtm2 | ENSMUSG00000071862.2 | M35 |
| | Rprm | ENSMUSG00000075334.2 | M35 |
| | Stmn3 | ENSMUSG00000027581.12 | M35 |
| | Ncald | ENSMUSG00000051359.10 | M35 |
| 5aj | Ttc9b | ENSMUSG00000007944.7 | M36 |
| | Nptx1 | ENSMUSG00000025582.4 | M36 |
| | Ogfrl1 | ENSMUSG00000026158.7 | M36 |
| | Tbr1 | ENSMUSG00000035033.11 | M36 |
| | Ipcef1 | ENSMUSG00000064065.11 | M36 |
| | Hs3st4 | ENSMUSG00000078591.1 | M36 |
| | Mctp1 | ENSMUSG00000021596.12 | M36 |
| 5ak | Ckb | ENSMUSG00000001270.8 | M37 |
| | Gstm1 | ENSMUSG00000058135.8 | M37 |
| | Sdc4 | ENSMUSG00000017009.3 | M37 |
| | Fabp7 | ENSMUSG00000019874.7 | M37 |
| | Id2 | ENSMUSG00000020644.8 | M37 |
| | Id4 | ENSMUSG00000021379.1 | M37 |
| | Glud1 | ENSMUSG00000021794.11 | M37 |
| | Clu | ENSMUSG00000022037.10 | M37 |
| | Tmem47 | ENSMUSG00000025666.12 | M37 |
| | Myoc | ENSMUSG00000026697.10 | M37 |
| | Aldh1l1 | ENSMUSG00000030088.11 | M37 |
| | Ttyh1 | ENSMUSG00000030428.12 | M37 |
| | Mt3 | ENSMUSG00000031760.8 | M37 |
| | Mt2 | ENSMUSG00000031762.6 | M37 |
| | Mt1 | ENSMUSG00000031765.7 | M37 |
| | Chst2 | ENSMUSG00000033350.7 | M37 |
| | Mmd2 | ENSMUSG00000039533.7 | M37 |
| | Mlc1 | ENSMUSG00000035805.9 | M37 |
| | Sfxn5 | ENSMUSG00000033720.8 | M37 |
| | Fbxo2 | ENSMUSG00000041556.8 | M37 |
| | Asrgl1 | ENSMUSG00000024654.8 | M37 |
| | Gfap | ENSMUSG00000020932.10 | M37 |
| | Aqp4 | ENSMUSG00000024411.9 | M37 |
| | Slc1a2 | ENSMUSG00000005089.11 | M37 |
| | Atp1a2 | ENSMUSG00000007097.10 | M37 |
| | Bcan | ENSMUSG00000004892.9 | M37 |
| | Aldoc | ENSMUSG00000017390.11 | M37 |
| | Ntsr2 | ENSMUSG00000020591.10 | M37 |
| | Ndrg2 | ENSMUSG00000004558.10 | M37 |
| | Slc4a4 | ENSMUSG00000060961.10 | M37 |
| | Ednrb | ENSMUSG00000022122.10 | M37 |
| | Prdx6 | ENSMUSG00000026701.11 | M37 |
| 5al | Gsn | ENSMUSG00000026879.10 | M38 |
| | Grb14 | ENSMUSG00000026888.10 | M38 |
| | Lpar1 | ENSMUSG00000038668.10 | M38 |
| | Tmem125 | ENSMUSG00000050854.5 | M38 |
| | Opalin | ENSMUSG00000050121.8 | M38 |
| | Ermn | ENSMUSG00000026830.9 | M38 |
| | Mog | ENSMUSG00000076439.8 | M38 |
| | Pdlim2 | ENSMUSG00000022090.6 | M38 |
| | Mag | ENSMUSG00000036634.11 | M38 |
| 5am | Hapln2 | ENSMUSG00000004894.6 | M39 |
| | Ndrg1 | ENSMUSG00000005125.8 | M39 |
| | Tppp3 | ENSMUSG00000014846.8 | M39 |
| | Qdpr | ENSMUSG00000015806.8 | M39 |
| | Aspa | ENSMUSG00000020774.5 | M39 |
| | Sec11c | ENSMUSG00000024516.8 | M39 |
| | Fth1 | ENSMUSG00000024661.6 | M39 |
| | Pla2g16 | ENSMUSG00000060675.9 | M39 |
| | Gatm | ENSMUSG00000027199.10 | M39 |
| | Mal | ENSMUSG00000027375.10 | M39 |
| | Car2 | ENSMUSG00000027562.8 | M39 |
| | Cryab | ENSMUSG00000032060.9 | M39 |
| | Fez1 | ENSMUSG00000032118.11 | M39 |
| | Trf | ENSMUSG00000032554.11 | M39 |
| | Cmtm5 | ENSMUSG00000040759.8 | M39 |
| | Fa2h | ENSMUSG00000033579.12 | M39 |
| | Anln | ENSMUSG00000036777.7 | M39 |
| | Rnf13 | ENSMUSG00000036503.9 | M39 |
| | Ppp1r14a | ENSMUSG00000037166.4 | M39 |
| | Gpr37 | ENSMUSG00000039904.8 | M39 |
| | Serpinb1a | ENSMUSG00000044734.11 | M39 |
| | Tmem88b | ENSMUSG00000073680.2 | M39 |
| | Evi2a | ENSMUSG00000078771.6 | M39 |
| | Plekhb1 | ENSMUSG00000030701.12 | M39 |
| | Apod | ENSMUSG00000022548.10 | M39 |
| | Gjb1 | ENSMUSG00000047797.10 | M39 |
| | Gm21984 | ENSMUSG00000095334.2 | M39 |
| 5an | Gstm5 | ENSMUSG00000004032.6 | M40 |
| | Sparcl1 | ENSMUSG00000029309.3 | M40 |
| | Ezr | ENSMUSG00000052397.8 | M40 |
| | Pantr1 | ENSMUSG00000060424.10 | M40 |
| | Acsl3 | ENSMUSG00000032883.11 | M40 |
| 5ao | Serping1 | ENSMUSG00000023224.8 | M41 |
| | Slc13a4 | ENSMUSG00000029843.4 | M41 |
| | Aldh1a2 | ENSMUSG00000013584.5 | M41 |
| | Gjb2 | ENSMUSG00000046352.7 | M41 |
| | Col3a1 | ENSMUSG00000026043.14 | M41 |
| | Aebp1 | ENSMUSG00000020473.9 | M41 |
| 5ap | Syngr1 | ENSMUSG00000022415.8 | M42 |
| | Celf4 | ENSMUSG00000024268.11 | M42 |
| | Napb | ENSMUSG00000027438.10 | M42 |
| | Sh3gl2 | ENSMUSG00000028488.11 | M42 |
| | Pgm2l1 | ENSMUSG00000030729.12 | M42 |
| | Ywhag | ENSMUSG00000051391.8 | M42 |
| | Basp1 | ENSMUSG00000045763.7 | M42 |
| | Syt1 | ENSMUSG00000035864.10 | M42 |
| | Prkcb | ENSMUSG00000052889.7 | M42 |
| | Chn1 | ENSMUSG00000056486.13 | M42 |
| | Lingo1 | ENSMUSG00000049556.4 | M42 |
| | Thy1 | ENSMUSG00000032011.4 | M42 |
| | Syn1 | ENSMUSG00000037217.11 | M42 |
| | Bsn | ENSMUSG00000032589.10 | M42 |
| | 6330403A02Rik | ENSMUSG00000053963.6 | M42 |
| 5aq | Gabra1 | ENSMUSG00000010803.8 | M43 |
| | Rgs7bp | ENSMUSG00000021719.8 | M43 |
| | Kcnh7 | ENSMUSG00000059742.6 | M43 |
| | Scn8a | ENSMUSG00000023033.10 | M43 |
| | Fam155a | ENSMUSG00000079157.3 | M43 |
| | Grin2a | ENSMUSG00000059003.8 | M43 |
| | Grm5 | ENSMUSG00000049583.10 | M43 |
| | Lphn1 | ENSMUSG00000013033.12 | M43 |
| 5ar | Hspb2 | ENSMUSG00000038086.3 | M44 |
| | Gja4 | ENSMUSG00000050234.7 | M44 |
| | Pde5a | ENSMUSG00000053965.6 | M44 |
| | Olfr558 | ENSMUSG00000070423.3 | M44 |
| | Gm13861 | ENSMUSG00000085382.1 | M44 |
| 5as | Alcam | ENSMUSG00000022636.9 | M45 |
| | Pvrl3 | ENSMUSG00000022656.11 | M45 |
| | Itpka | ENSMUSG00000027296.7 | M45 |
| | Pcsk2 | ENSMUSG00000027419.9 | M45 |
| | Calb1 | ENSMUSG00000028222.2 | M45 |
| | Cx3cl1 | ENSMUSG00000031778.8 | M45 |
| | Wfs1 | ENSMUSG00000039474.9 | M45 |
| | Gucy1a3 | ENSMUSG00000033910.9 | M45 |
| | Arf3 | ENSMUSG00000051853.8 | M45 |
| | Rbfox1 | ENSMUSG00000008658.11 | M45 |
| | Mapk1 | ENSMUSG00000063358.11 | M45 |
| | Meis2 | ENSMUSG00000027210.16 | M45 |
| | Tsnax | ENSMUSG00000056820.6 | M45 |
| | Cacng3 | ENSMUSG00000066189.5 | M45 |
| | Ppp3r1 | ENSMUSG00000033953.10 | M45 |
| | Megf9 | ENSMUSG00000039270.5 | M45 |
| | Cux2 | ENSMUSG00000042589.14 | M45 |
| | Rasgrf2 | ENSMUSG00000021708.12 | M45 |
| 5at | Stx1a | ENSMUSG00000007207.6 | M46 |
| | Ywhah | ENSMUSG00000018965.10 | M46 |
| | Rtn1 | ENSMUSG00000021087.13 | M46 |
| | Gfra2 | ENSMUSG00000022103.9 | M46 |
| | Grm2 | ENSMUSG00000023192.9 | M46 |
| | Epha4 | ENSMUSG00000026235.10 | M46 |
| | Cnih3 | ENSMUSG00000026514.9 | M46 |
| | Rgs4 | ENSMUSG00000038530.7 | M46 |
| | Hpca | ENSMUSG00000028785.8 | M46 |
| | Atp1a1 | ENSMUSG00000033161.9 | M46 |
| | Nrn1 | ENSMUSG00000039114.11 | M46 |
| | Camk4 | ENSMUSG00000038128.6 | M46 |
| | Zdhhc2 | ENSMUSG00000039470.11 | M46 |
| | Stxbp1 | ENSMUSG00000026797.11 | M46 |
| | Camk2n1 | ENSMUSG00000046447.3 | M46 |
| | Hrh3 | ENSMUSG00000039059.7 | M46 |
| | Nrsn1 | ENSMUSG00000048978.9 | M46 |
| | Ncam2 | ENSMUSG00000022762.13 | M46 |
| | Syp | ENSMUSG00000031144.11 | M46 |
| | Kcnq3 | ENSMUSG00000056258.8 | M46 |
| | Gabrg2 | ENSMUSG00000020436.13 | M46 |
| | Negr1 | ENSMUSG00000040037.9 | M46 |
| | Fat3 | ENSMUSG00000074505.4 | M46 |
| | Nol4 | ENSMUSG00000041923.11 | M46 |
| | AI593442 | ENSMUSG00000078307.2 | M46 |
| | Cacnb4 | ENSMUSG00000017412.11 | M46 |
| | Nsf | ENSMUSG00000034187.14 | M46 |
| | Car10 | ENSMUSG00000056158.10 | M46 |
| | Vstm2l | ENSMUSG00000037843.6 | M46 |
| | Slc24a3 | ENSMUSG00000063873.6 | M46 |
| | Foxp1 | ENSMUSG00000030067.13 | M46 |
| | Lmo4 | ENSMUSG00000028266.13 | M46 |
| | 6330403K07Rik | ENSMUSG00000018451.6 | M46 |
| | Nrxn1 | ENSMUSG00000024109.14 | M46 |
| | Mef2c | ENSMUSG00000005583.12 | M46 |
| 5au | Hspb1 | ENSMUSG00000004951.10 | M47 |
| | Wfdc1 | ENSMUSG00000023336.5 | M47 |
| | Sema3c | ENSMUSG00000028780.9 | M47 |
| | Pglyrp1 | ENSMUSG00000030413.6 | M47 |
| | Palmd | ENSMUSG00000033377.10 | M47 |
| | Vwa1 | ENSMUSG00000042116.3 | M47 |
| | Ablim1 | ENSMUSG00000025085.12 | M47 |
| | Emcn | ENSMUSG00000054690.13 | M47 |
| | Spock2 | ENSMUSG00000058297.12 | M47 |
| | Lmo2 | ENSMUSG00000032698.11 | M47 |
| 5av | Spp1 | ENSMUSG00000029304.10 | M48 |
| | Tbx18 | ENSMUSG00000032419.8 | M48 |
| | Lum | ENSMUSG00000036446.4 | M48 |
| | Itih2 | ENSMUSG00000037254.14 | M48 |
| | Col13a1 | ENSMUSG00000058806.10 | M48 |
| | Cped1 | ENSMUSG00000062980.11 | M48 |
| 5aw | Slc47a1 | ENSMUSG00000010122.10 | M49 |
| | Ogn | ENSMUSG00000021390.5 | M49 |
| | 1500015O10Rik | ENSMUSG00000026051.8 | M49 |
| | Slc26a7 | ENSMUSG00000040569.9 | M49 |
| | Prg4 | ENSMUSG00000006014.12 | M49 |
| | Islr | ENSMUSG00000037206.10 | M49 |
| 5ax | Nrip3 | ENSMUSG00000034825.9 | M50 |
| | Cnr1 | ENSMUSG00000044288.6 | M50 |
| | Tcf4 | ENSMUSG00000053477.11 | M50 |
| | Igf1 | ENSMUSG00000020053.14 | M50 |
| | Erbb4 | ENSMUSG00000062209.11 | M50 |
| | Gad1 | ENSMUSG00000070880.6 | M50 |
| | Adarb2 | ENSMUSG00000052551.11 | M50 |
| | Rab3c | ENSMUSG00000021700.9 | M50 |
| 5ay | Efr3a | ENSMUSG00000015002.12 | M51 |
| | Impact | ENSMUSG00000024423.5 | M51 |
| | Serpini1 | ENSMUSG00000027834.11 | M51 |
| | Pcp4 | ENSMUSG00000090223.1 | M51 |
| | Olfm3 | ENSMUSG00000027965.11 | M51 |
| | Mdh1 | ENSMUSG00000020321.11 | M51 |
| | Myl4 | ENSMUSG00000061086.8 | M51 |
| | Dync1i1 | ENSMUSG00000029757.12 | M51 |
| | Rgs17 | ENSMUSG00000019775.13 | M51 |
| 5az | Kcnv1 | ENSMUSG00000022342.5 | M52 |
| | Ppp1r1a | ENSMUSG00000022490.6 | M52 |
| | Stmn2 | ENSMUSG00000027500.10 | M52 |
| | Gnao1 | ENSMUSG00000031748.11 | M52 |
| | Cpne4 | ENSMUSG00000032564.11 | M52 |
| | Rasgrp1 | ENSMUSG00000027347.14 | M52 |
| | Gap43 | ENSMUSG00000047261.9 | M52 |
| | Gng2 | ENSMUSG00000043004.9 | M52 |
| 5ba | Fli1 | ENSMUSG00000016087.9 | M53 |
| | Srgn | ENSMUSG00000020077.10 | M53 |
| | Fam101b | ENSMUSG00000020846.6 | M53 |
| | Itih5 | ENSMUSG00000025780.7 | M53 |
| | Slc40a1 | ENSMUSG00000025993.6 | M53 |
| | Slc6a6 | ENSMUSG00000030096.7 | M53 |
| | Slc38a5 | ENSMUSG00000031170.10 | M53 |
| | Nostrin | ENSMUSG00000034738.8 | M53 |
| | Slc16a1 | ENSMUSG00000032902.1 | M53 |
| | Eltd1 | ENSMUSG00000039167.7 | M53 |
| | Arl4a | ENSMUSG00000047446.14 | M53 |
| | Egfl7 | ENSMUSG00000026921.14 | M53 |
| | Car4 | ENSMUSG00000000805.14 | M53 |
| | Kdr | ENSMUSG00000062960.7 | M53 |
| | Abcg2 | ENSMUSG00000029802.9 | M53 |
| | Myl12a | ENSMUSG00000024048.10 | M53 |
| | Bsg | ENSMUSG00000023175.11 | M53 |

In one example, the plurality of pre-determined genes is expressed in the digestive tract. In a further example, the pre-determined genes are expressed in the intestinal cells. In a further example, the plurality of pre-determined genes is expressed in cells associated with colorectal cancer. In some examples, the cells can include, but are not limited to epithelial cells, CAF-1 cells, immune cells and CAF-2 cells. In another example, the plurality of pre-determined genes expressed in epithelial cells include genes listed in Table 6 (6a). In another example, the plurality of pre-determined genes expressed in CAF-1 cells include genes listed in Table 6 (6b). In another example, the plurality of pre-determined genes expressed in immune cells include genes listed in Table 6 (6c). In another example, the plurality of pre-determined genes expressed in CAF-2 cells include genes listed in Table 6 (6d). As exemplified in FIG. 19B, the method as described herein identified distinct spatial organization of the two CAF subtypes, demonstrating the specificity and sensitivity of the ISH method for cell heterogeneity characterisation.

TABLE 6
FISHnCHIPs for FIG. 19 Human Colorectal Cancer Library

| Table ID | Gene | Transcript ID | Cell type |
|---|---|---|---|
| 6a | TMEM54 | uc001bwi.1 | Epithelial |
| | TSPAN1 | uc009vyd.1 | Epithelial |
| | ELF3 | uc001gxh.3 | Epithelial |
| | PIGR | uc001hez.2 | Epithelial |
| | EPCAM | uc002rvx.2 | Epithelial |
| | FABP1 | uc002sst.1 | Epithelial |
| | CLDN3 | uc003tzg.3 | Epithelial |
| | KRT8 | uc001sbd.2 | Epithelial |
| | KRT18 | uc001sbg.2 | Epithelial |
| | TSPAN8 | uc009zrt.1 | Epithelial |
| | PHGR1 | uc010uco.1 | Epithelial |
| | CLDN7 | uc002gfm.3 | Epithelial |
| | FXYD3 | uc002nxv.2 | Epithelial |
| | CEACAM5 | uc002or1.2 | Epithelial |
| | CLDN4 | uc003tzi.3 | Epithelial |
| | LGALS4 | uc002ojg.2 | Epithelial |
| | KRT19 | uc002hxd.3 | Epithelial |
| | CEACAM6 | uc002orm.2 | Epithelial |
| 6b | DPT | uc001gfp.2 | CAF-1 |
| | COL3A1 | uc002uqj.1 | CAF-1 |
| | COL5A2 | uc002uqk.2 | CAF-1 |
| | COL6A3 | uc010znj.1 | CAF-1 |
| | CCDC80 | uc003dzg.2 | CAF-1 |
| | ADH1B | uc003hus.3 | CAF-1 |
| | SFRP2 | uc003inv.1 | CAF-1 |
| | AEBP1 | uc003tkb.2 | CAF-1 |
| | COL1A2 | uc003ung.1 | CAF-1 |
| | PCOLCE | uc003uvo.2 | CAF-1 |
| | SFRP1 | uc003xnt.2 | CAF-1 |
| | C1S | uc001qsl.2 | CAF-1 |
| | C1R | uc010sfy.1 | CAF-1 |
| | LUM | uc001tbm.2 | CAF-1 |
| | DCN | uc001tbt.2 | CAF-1 |
| | MFAP4 | uc002gvt.2 | CAF-1 |
| | COL1A1 | uc002iqm.2 | CAF-1 |
| | COL6A1 | uc002zhu.1 | CAF-1 |
| | OGN | uc004asa.2 | CAF-1 |
| | FBLN1 | uc003bgj.1 | CAF-1 |
| | COL5A1 | uc004cfe.2 | CAF-1 |
| | COL6A2 | uc002zia.1 | CAF-1 |
| | THY1 | uc001pwq.2 | CAF-1 |
| | MMP2 | uc010vhd.1 | CAF-1 |
| 6c | CD74 | uc003lsd.2 | Immune |
| | HLA-DRA | uc003obh.2 | Immune |
| | HLA-DRB1 | uc003obp.3 | Immune |
| | HLA-DQA1 | uc003obr.2 | Immune |
| | HLA-DQB1 | uc003obw.2 | Immune |
| | HLA-DPA1 | uc003ocs.1 | Immune |
| | HLA-DPB1 | uc003ocu.1 | Immune |
| 6d | ACTA2 | uc001kfp.2 | CAF-2 |
| | TAGLN | uc001pqm.2 | CAF-2 |
| | MYL9 | uc002xfl.1 | CAF-2 |

While Tables 2-6 provide exemplary panels of genes to be targeted in the in situ hybridisation method as described herein in kidney, brain, and digestive tract, a person skilled in the art can appreciate that the panel of genes are identified based on the purpose of the experiment. Therefore, the method as described herein is not limited by the exemplary panels listed. Alternative panels can be obtained in accordance with the method as described herein based on user defined cell types (for cell-centric strategy) or selected gene expression programs (for gene-centric strategy).

The method as described herein is useful for the profiling of the cell types within a biological sample, for the identification of novel cell types, and for the validation of novel cell types identified from scRNA-seq studies. For example, FIG. 13 provides large Field of View (FOV) in situ hybridisation using the gene-centric strategy as described herein. As shown in the UMAP of FIG. 13A (right), an unknown cell cluster has been identified independent from other cell types.

Similar to conventional methods such as multiplexed single molecule FISH (smFISH), the in situ hybridisation method can be used to quantify cell types, derive zonation patterns, and analyse cell-cell interactions. Spatial patterns of signal intensities can be uncovered using the method as described herein, as described in FIG. 11A, for example. FIG. 11A shows gradual intensity variation along the cortical depth within the mouse brain cortex for some of the gene expression programs. FIG. 19B demonstrates novel cell-cell interaction between immune cells and the cancer subtype cells cancer associated fibroblasts 1 (CAF-1) and cancer associated fibroblasts 2 (CAF-2), which are observed using the in situ hybridisation method described herein. The method as described herein provides robust and sensitive signal measurements at cell level by grouping multiple genes and labelling them together improves signal to noise. In addition, by combining the method described herein with multiplexed smFISH, transcriptomic information at both cell levels and transcript-level can be obtained simultaneously.

The sensitivity of the method as described herein allows the simpler, faster and lower instrument cost for spatial transcriptomics, thereby improving the accessibility of spatial assays for the broader biomedical research. Besides neuroscience and oncology, the described method finds use in other biological studies, such as understanding spatial gene coordination during embryonic development or defining multi-cellular ecosystems of infectious pathogens. The method is useful for the molecular histopathology of Formalin Fixed Paraffin Embedded (FFPE) tissues, where clinically actionable cell states can be diagnosed accurately and at scale. Therefore, as described herein, the in situ hybridisation method is a sensitive, robust, and scalable spatial transcriptomics method that profiles single cells within a tissue sample.

In another aspect, the present disclosure provides a method of making/providing the prognosis for a subject suffering from cancer. The method comprises obtaining a sample of the subject. The sample can be, but is not limited to, a biopsy sample obtained from the subject, or a tissue sample obtained from cancer tissue. The method further comprises characterizing one or more cancer cells in the sample using the method as described herein to determine the stage of the cancer. Methods and criteria for determining the stages of a cancer have been well established in the art. For example, the TNM Staging System is the most commonly used staging system used by healthcare professionals. Typically, TNM Staging System comprises three dimensions: T is used to describe the size of the tumor (T1-T4); N is used to describe the presence of cancer in lymph nodes (N0-N3), and lastly, M represents the metastasis of cancer (M0 or M1). Alternatively, under number staging system, the development of cancers comprises five stages, i.e., Stage 0: cancer in situ; Stage I: early-stage cancer; Stage II and III: cancer spreading to nearby tissue; and Stage IV: metastatic cancer. The different stages of the cancers can be differentiated by profiling the gene expression of cells within the tissue at each stage. A person skilled in the art would be able to determine the stages of cancer based on suitable information revealed from the method a biological sample, such as a biopsy sample. In a further example, the method comprises determining the prognosis based on the stage of the cancer.

In another aspect, the present disclosure provides a kit for characterizing cells in a biological sample in situ. The kit comprises a plurality of probes that bind to ribonucleic acid (RNA) transcripts of a plurality of pre-determined genes as described herein. In one example, each probe comprises a detectable label. In another example, each probe comprises a domain that binds specifically to a ribonucleic acid transcript of one of the pre-determined genes as described herein. In a further example, the kit comprises instructions for use.

In another example of the kit as described herein, the plurality of pre-determined genes comprises at least one gene and at least one other gene that are co-regulated, wherein the at least one gene and the at least one other gene are markers of a specific cell type, differentially expressed genes of a specific cell type, markers of a gene expression program or a gene regulatory module, markers of a biological pathway, or a combination thereof. In a further example, the at least one other gene is selected from one or more input datasets. Suitable input datasets can be selected based on the experimental design by a person skilled in the art, which include but are not limited to: a bulk RNA sequencing, a single-cell RNA sequencing, a microarray dataset, a chromatin accessibility sequencing, a methylation sequencing, a DNA-associated proteins sequencing, a spatial transcriptomics sequencing, a multiplexed RNA fluorescence in situ hybridisation, a multiplexed immunohistochemistry, a bioinformatics database, or any user-defined dataset or combinations thereof. In another example, the bioinformatics database used to obtain sets of pre-determined genes is selected from the group consisting of Kyoto Encyclopedia of Genes and Genomes (KEGG) or Panther or Database for Annotation, Visualization, and Integrated Discovery (DAVID) or Gene Ontology (GO) or combinations thereof. Additionally, prior knowledge on biochemical pathways, transcription factors, or cis-regulatory sequences can be incorporated as part of the input. Based on the input dataset of pre-determined genes, a person skilled in the art would be able to calculate, with existing mathematical tools, whether two genes are likely to show coordinated change in expression levels within a cell.

In one example of the kit as described herein, the plurality of pre-determined genes is expressed in kidney, brain, or the digestive tract. In another example, the plurality of pre-determined genes is expressed in cancer tissues. In a further example, the plurality of pre-determined genes is selected from the genes listed in Table 2 (2a)-(2e), Table 3 (3a)-(3r), Table 4 (4a)-(4t), Table 5 (5a)-(5ba), and Table 6 (6a)-(6d).

In another aspect, the present disclosure provides a kit for characterizing a colorectal cancer in situ. In one example, the kit comprises a plurality of probes that bind to ribonucleic acid (RNA) transcripts of a plurality of pre-determined genes as described herein. In another example, the plurality of pre-determined genes is selected from genes listed in Table 6 (6a)-(6d). In a further example, each probe of the plurality of probes comprises a detectable label as described herein. In a further example, each probe of the plurality of probes comprises a domain that binds specifically to a ribonucleic acid transcript of the plurality of pre-determined genes as described herein. In another example, the kit further comprises instructions for use.

The disclosure has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein. Other embodiments are within the following claims and non-limiting examples. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

### Experimental Section

### Gene Panel Design and Evaluation Software

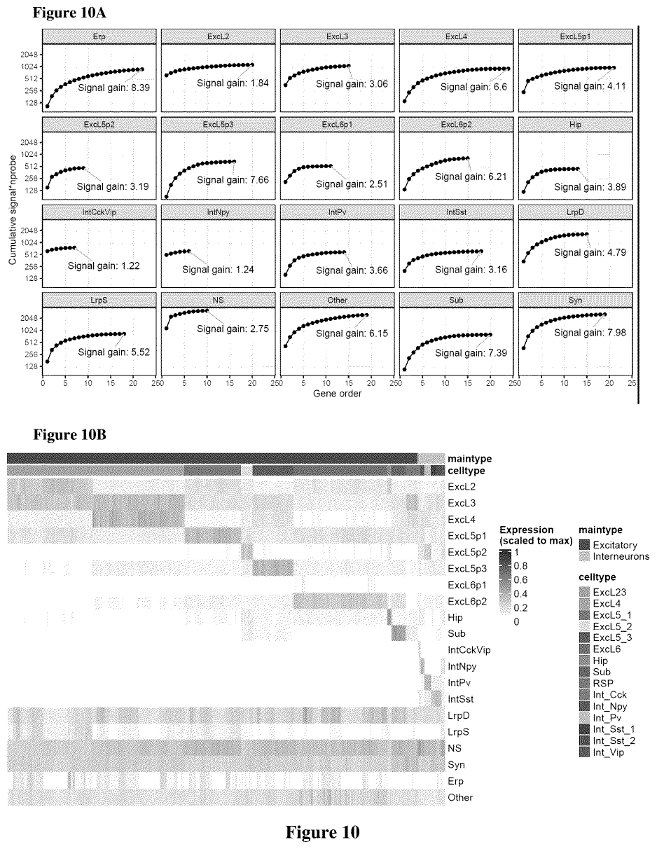

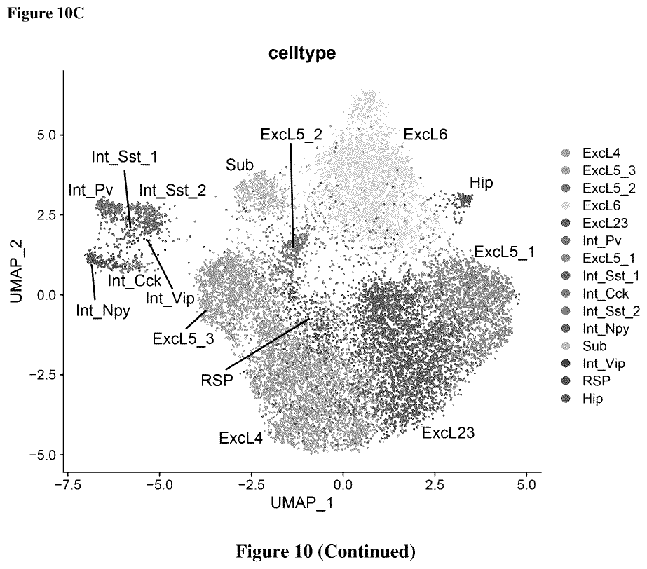

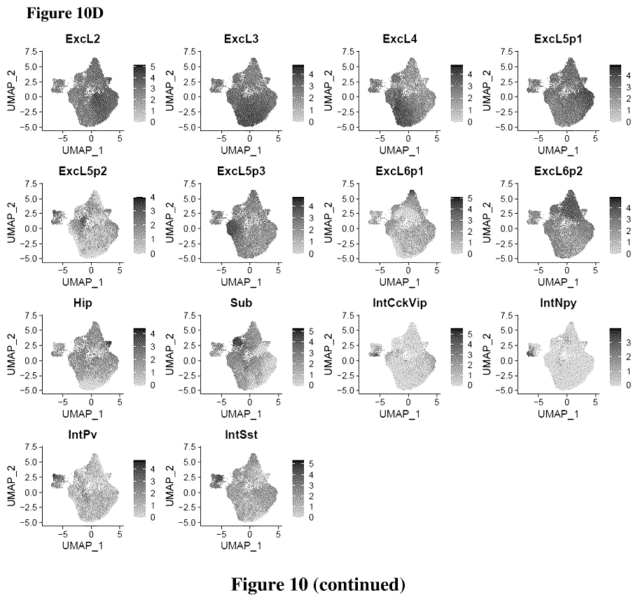

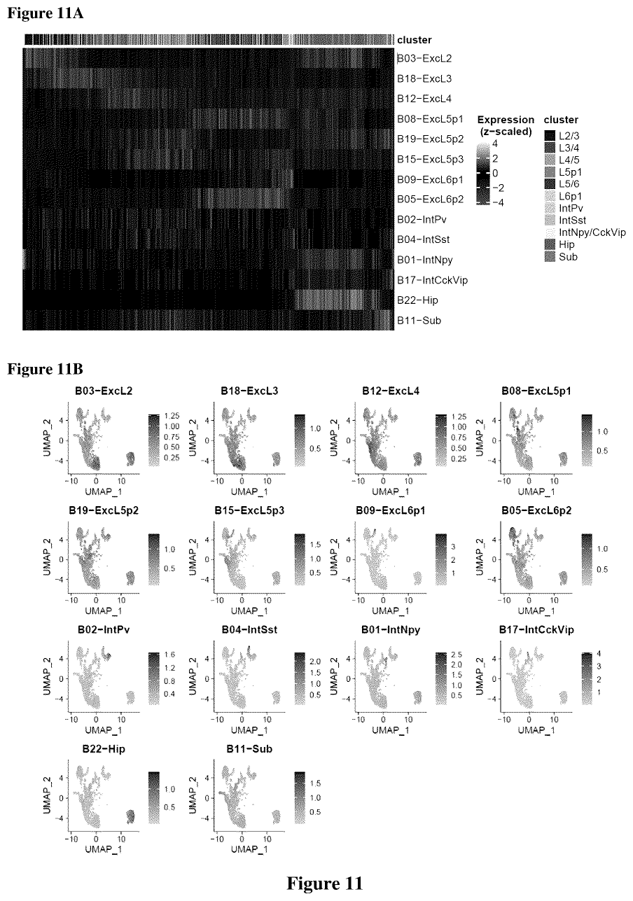

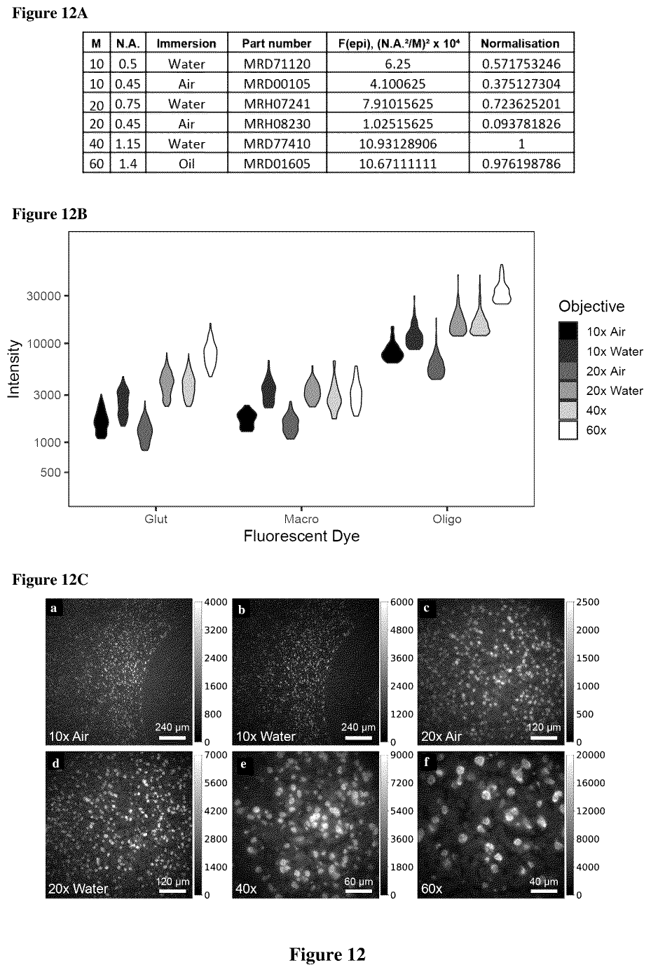

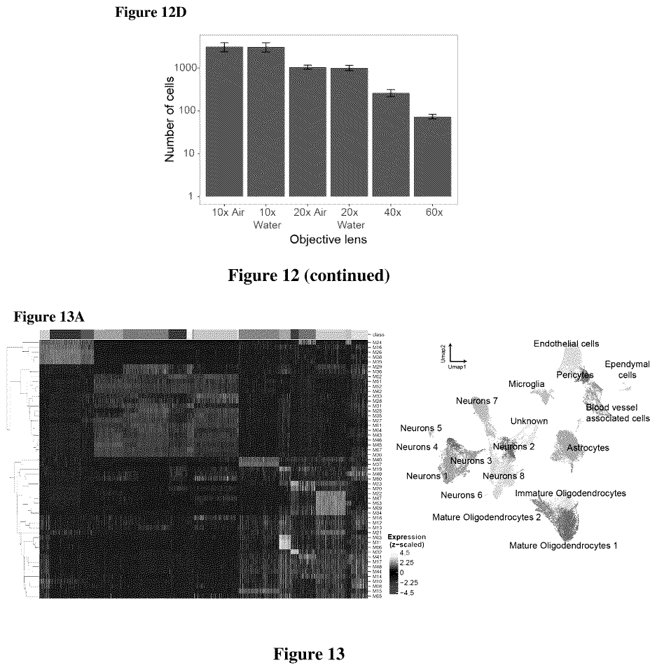

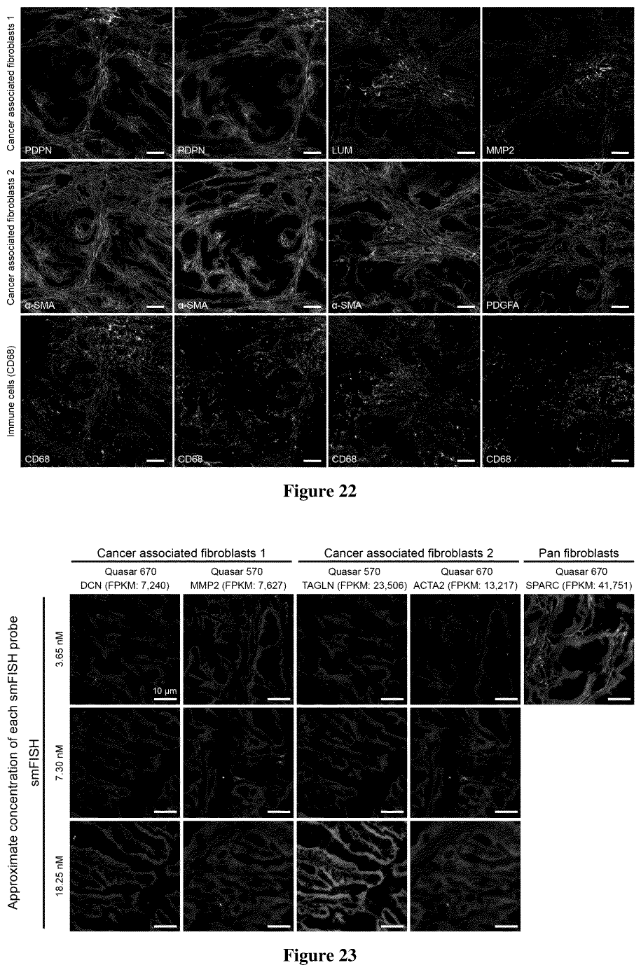

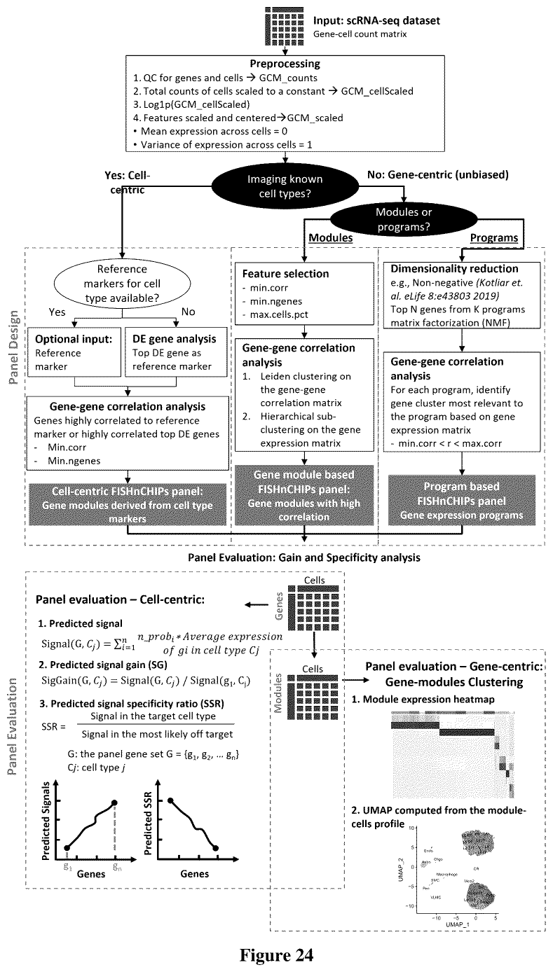

The software workflow for the in situ hybridisation panel design and evaluation is summarized in FIG. 24. To target specific cell types, cell-centric strategy of the in situ hybridisation method described herein either accepts user input of reference markers and cell labels or performs de novo clustering of cell types and identifies Differentially Expressed (DE) gene(s) as the reference marker(s). The default measure of correlation is the Pearson's correlation coefficient. Other possible measures include mutual information, Spearman's rank correlation coefficient, and Euclidean distance. To explore gene expression activities without a priori cell type clustering of the scRNA-seq data, the gene-centric in situ hybridisation method performs either feature selection and/or dimensionality reduction (for example, using non-negative matrix factorization (NMF)), followed by clustering analysis of the gene-gene correlation matrix to identify gene modules. In the feature gene module-based method, genes that were highly correlated (>min. corr) with a minimum number of genes (>min. genes) were used as nodes in a network that was constructed from the gene-gene correlation matrix and partitioned using the Leiden algorithm. Gene partitions can be further sub-clustered using hierarchical clustering based on their log-transformed expression matrix. For the dimensionality reduction-based method, a non-negative matrix factorization (NMF) algorithm that identifies gene programs and their relative contributions can be used. The top N genes from each program are chosen to construct the gene-gene correlation matrix. Clustering of the matrices can be refined by setting correlation ranges. A hybrid in situ hybridisation method is also designed where the Differentially Expressed (DE) genes are used as features to construct the gene-gene correlation matrix to identify gene modules. Users are recommended to perform clustering in the gene-gene space to reduce crosstalk. The output gene panel is evaluated by predicting the signal gain and specificity, as well as by simulating the expected cell-module expression profile and clusters. The present application provides demonstration of cell-centric in situ hybridisation for the mouse kidney library (FIGS. 2-4), gene-centric in situ hybridisation for the mouse cortex libraries (FIGS. 5-11), and hybrid approach for the mouse brain (FIGS. 12-18) and human CRC library (FIGS. 19-23).

The following paragraphs describe the in situ hybridisation panel design and evaluation process in more detail:

### Data Pre-Processing

The scRNA-seq count matrix is pre-processed using the Seurat pipeline. First, the quality control (QC) filters empty droplets and cell doublets, i.e., cells expressing too few or too many unique genes. After QC, three versions of the gene-count matrix will be prepared for different downstream analyses: 1) Scale the total counts of cells to a constant by dividing the total counts of cells and multiplying a scale factor. The cell-scaled matrix would be used for predicting the expected signal of an in situ hybridisation panel; 2) Add a pseudo-count to the cell-scaled matrix and apply a natural log transformation. The log-transformed matrix would be used for the differential gene analysis and gene-gene correlation analysis; 3) Apply a linear transformation to the gene expression vectors, so that the mean expression of genes across cells is 0 and the variance across cells is 1. The gene-scaled matrix would be used for dimensionality reduction and heatmap visualization of the expression of individual genes.

### Panel Evaluation

An in situ hybridisation panel can be evaluated by the signal gain and signal specificity ratio:
Denoting an in situ hybridisation panel with n genes as Pₜ={g₁, g₂, . . . , gᵢ, . . . , gₙ} targeting the cell type Cₜ;
the number of probes for genes corresponds to K={k₁, k₂, . . . , kᵢ, . . . , kₜ}.
The predicted signal of one gene gᵢ in cell type Cₜ, denoted as signal (gᵢ, Cₜ), is defined as the product of kᵢ and the average expression of gᵢ in cell type Cₜ.
The signal of a panel Pₜ in a cell type Cₜ, which is denoted as signal (Pₜ, Cₜ), is the sum of all gene signals in the target cell type or module.
Denoting g₁as the reference gene, and gₘₐₓ as the gene with the maximal signal.
The general signal gain is defined as

$$\frac{signal ( {P}_{t} , {C}_{t} )}{signal ( {g}_{1} , {C}_{t} )} ,$$

i.e., the ratio of the panel signal to the signal of the reference gene.
The conservative signal gain is defined as

$$\frac{signal ( {P}_{t} , {C}_{t} )}{signal ( {g}_{max} , {C}_{t} )} ,$$

i.e., the ratio of the panel signal to the highest gene signal.
The cross-talk can be estimated by calculating the signal specificity ratio of a panel Pₜ, between cell type Cₜ and

$${C}_{t}^{′} ,$$

defined as

$$\frac{signal ( {P}_{t} , {C}_{t}^{′} )}{signal ( {P}_{t} , {C}_{t} )} ,$$

i.e., ratio of panel signal in Cₜ to the ratio of panel signal in

$${C}_{t}^{′} .$$

The general signal specificity is defined as the ratio of the panel signal in the target cell type to the panel signal in all off-target cell types. The conservative signal specificity is defined as the ratio of the panel signal in the target cell type to the panel signal in the cell cluster with the highest predicted crosstalk. The general signal gain is used for the cell-centric mouse kidney panel and the conservative signal gain for all other in situ hybridisation panels. An in situ hybridisation panel can be further evaluated by re-clustering the scRNA-seq dataset using the module-cell expression matrix. The module-cell expression matrix is calculated from the cell-scaled expression matrix, by taking the sum of cell counts of genes in the same group. Considering the module as a meta-gene, the module-expression matrix can be taken as a meta-gene expression matrix. Consequently, conventional clustering methods used to process single-cell gene-count matrices can be applied. A module-cell expression heatmap and dimensionality-reduction visualization tools (such as UMAP or tSNE) could be used to simulate the reconstruction of cell types from the in situ hybridisation assay described herein.

### Designing Cell-Centric Mouse Kidney Panel

The scRNA-seq data and cell labels of the mouse kidney were retrieved from NCBI Gene Expression Omnibus (GEO) under accession GSE115746. Genes with the highest log fold-change of the average expression between the targeting clusters and other clusters were selected as reference markers. Cells with <200 or >3000 unique expressed genes were removed. Cells with mitochondrial genes >50% were removed. Genes that were expressed in <10 cells were removed. Cells were then scaled to a sequence depth of 10,000 per cell and log-transformed with a pseudo-count of 1. Genes were scaled so that the mean expression across cells was 0 and the variance across cells is 1. For each cluster, genes correlated to the reference markers and with Pearson Correlation >0.5 were selected. If there were <15 genes highly correlated with the reference, the top 15 genes were selected. For all clusters, we removed genes that appeared more than once. For glomerular endothelial cells, the top maker Plat was only expressed in 59.5% of glomerular endothelial cells, and it was also highly expressed in glomerular podocytes. Therefore, Emcn was used as the reference marker instead of Plat. For renal macrophages, both Clqa and Clqb were used as references. As shown in FIG. 2, five cell types were used for imaging. However, all the previously annotated cell types have been computationally evaluated as detailed in FIG. 4.

### Designing Gene-Centric Mouse Cortex Panel

A scRNA-seq dataset of the mouse primary visual cortex (VISp) was used for the mouse brain panel design in relation to FIG. 5-FIG. 8. First, the cells were scaled to 10,000, then the gene expression in cells was binarized by the mean expression of all genes across all cells. Genes that were expressed in <5 cells or >80% of the total number of cells were filtered out. Gene names starting with “Mt” or “Gm” followed by digits were removed. 330 genes highly correlated to at least 5 genes with a correlation >0.7 were selected as candidates. A graph was created from the 330 by 330 correlation matrix, removing edges with low correlation (<0.6). Leiden partitioning on the graph with 330 candidate genes generated 11 clusters. Hierarchical clustering was performed on the Leiden clusters based on gene expression, cutting the dendrogram of genes into k subclusters: k=6 for big clusters (>30 genes); k=4 for mid-size clusters (11-30 genes); k=2 for small clusters (6-10 genes); k=1 for very small clusters (<6 genes). There were 255 genes distributed in 18 modules after removing subclusters with single genes, genes not found in our probe design transcriptome database (Hsp25-ps1 and Gstm2-ps1) or associated with multiple IDs in our probe design transcriptome database (Schip1). Functional enrichment analysis, known as gene set enrichment analysis, on the panel genes was performed using g:GOst.

### Dimensionality Reduction-Based Mouse Cortex Panel

Non-negative matrix factorization (NMF) provides a low rank approximation of the gene cell matrix by a product of two non-negative matrices, and is able to capture the structures of coordinated gene expression in scRNA-seq data. The gene-contribution matrix of the mouse visual cortex neurons was downloaded from Kotliar, D. et al. (Kotliar, D. et al. Identifying gene expression programs of cell-type identity and cellular activity with single-cell RNA-Seq. Elife 8, 1-26 (2019)). The highest contributing 50 genes were selected from the 20 factors. Gene names starting with the “Gm” followed by digits were removed. Clustering of the gene-gene correlation matrices resulted in one or more gene modules per program. As shown in FIG. 9-FIG. 11, by comparing the gene expression heatmap and the gene-gene correlation matrices, most genes with a Pearson's correlation (r) higher than 0.3 showed expression that spanned multiple programs and were markers associated with the major cell types (such as for all inhibitory neurons). Therefore, we removed genes with r higher than 0.3 and lower than 0.02. There were 311 genes distributed in 20 programs after further discarding genes with no probes found.

### 674-Gene Mouse Brain Panel

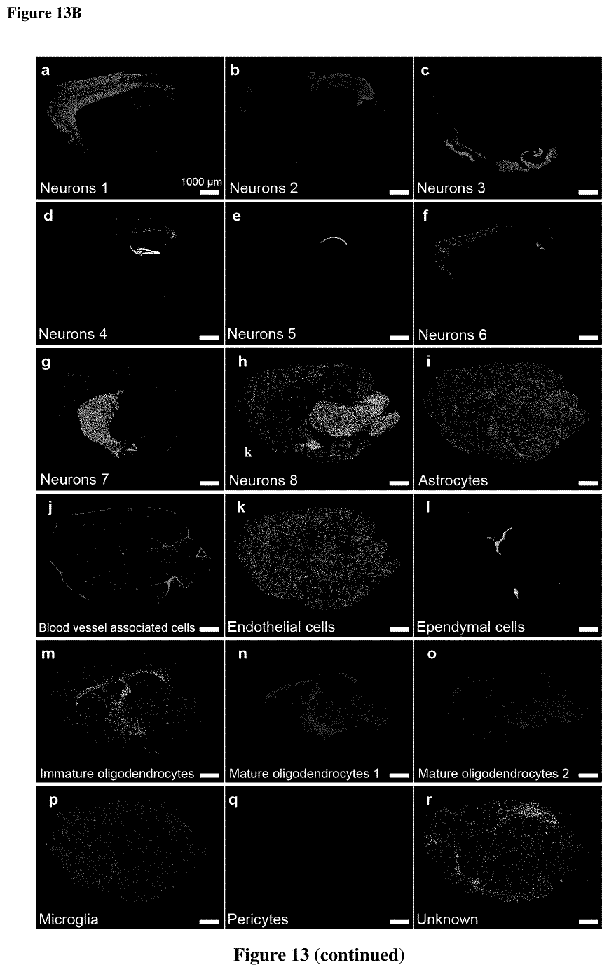

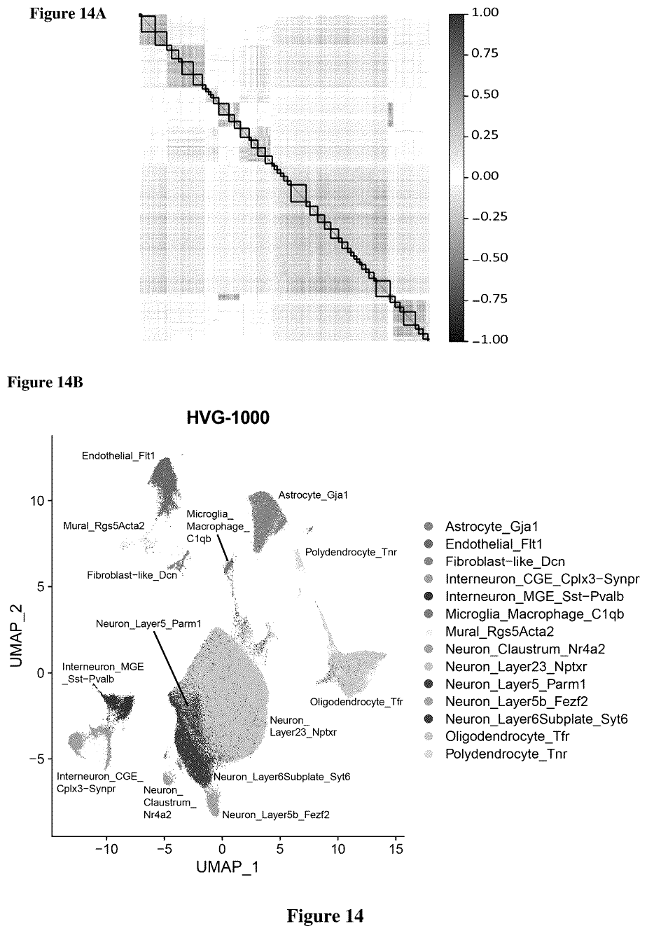

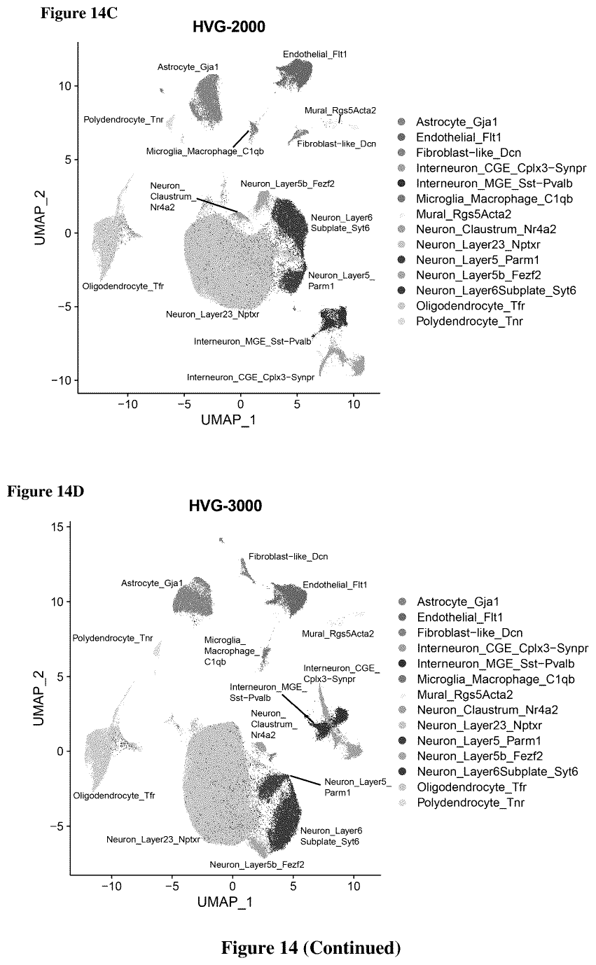

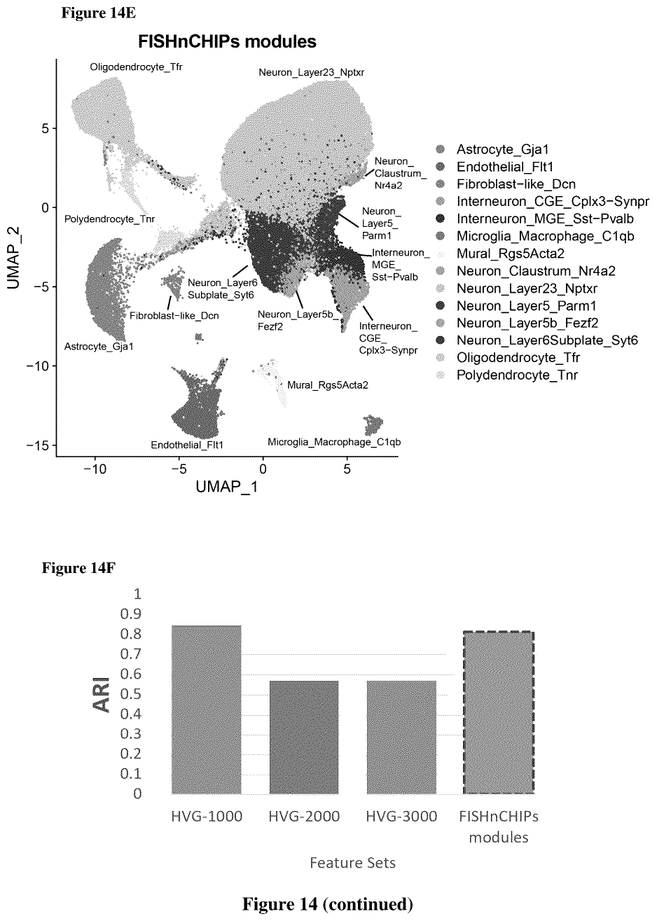

Utilizing the subcluster labels provided by the mouse brain Drop-seq scRNA dataset, a maximum of 50 Differentially Expressed (DE) genes were identified with at least 0.25-fold difference for all subclusters, employing the Wilcoxon Rank Sum test algorithm implemented in Seurat. For each subcluster, genes with the lowest correlation to any DE gene were removed until the minimal Pearson correlation matrix of the remaining genes was greater than 0.1. To further refine the quality of the panel, genes starting with ‘mt’ and small modules with fewer than 5 genes were excluded, resulting in 53 gene modules containing 674 genes. To evaluate the panel, the scRNA-seq dataset were re-clustered using the 53 modules as features and calculated the Adjusted Rand Index using the ‘aricode’ package in R. To provide further comparisons, single gene-based multiplexed FISH assays were also simulated by re-clustering the scRNA-seq data using 1000, 2000, and 3000 highly variable genes as features (FIG. 14).

### Human Colorectal Cancer (CRC) Panel

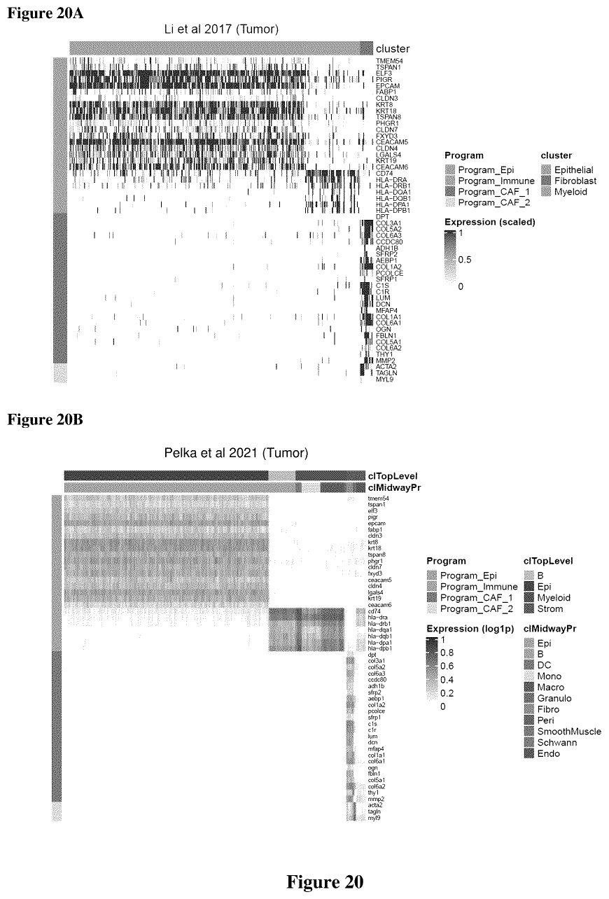

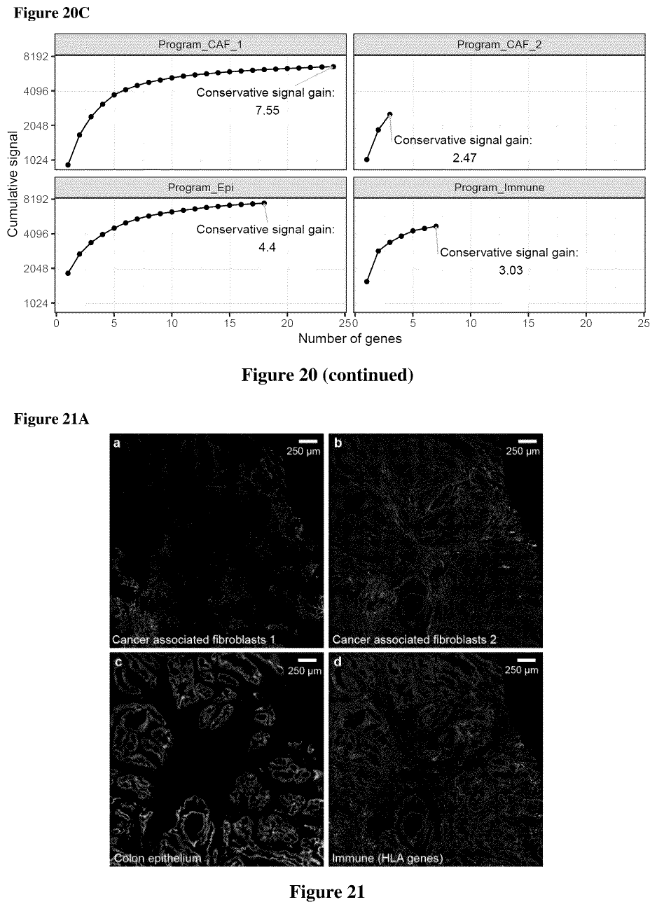

Two cancer-associated fibroblasts (CAFs) subtypes were previously identified using scRNA-seq. These two subtypes have been further confirmed using a more recent scRNA sequencing dataset (FIG. 20). Genes that were expressed in <5 cells or >70% of the total number of cells were filtered out. Gene names starting with “Rp”, “Mt” or “Gm” followed by digits were removed. Based on the 125 selected marker genes, a graph was created from the gene-gene correlation matrix, removing edges with low correlation (<0.7). Leiden partitioning on the graph yielded ~20 modules and we selected 4 modules highly expressed in the two CAFs, epithelial, and immune cells for demonstrating the in situ hybridisation method as described herein.

### The In Situ Hybridisation Library Design and Probe Sequences

For all the genes, 25-nucleotide target regions were identified using a previously published algorithm (DeTomaso, D. & Yosef, N., 2021). Briefly, reference transcript sequences were downloaded from the GENCODE website (human v24 and mouse m4). A specificity table was calculated using 15-nucleotide seed and 0.2 specificity cut-off was used. Quartet repeats (′AAAA′, ‘TTTT’, ‘GGGG’, and ‘CCCC’) were excluded from the possible target regions. A list of the readout probes sequences generated is shown in Table 1. A total of 56 readout probe sequences were generated initially, but B16, B48 and B55 were not used.

### Probe Amplification and Preparation

The probe library (Genscript) was amplified as described in a previously published protocol (Kuemmerle, L. B. et al. Probe set selection for targeted spatial transcriptomics. Bioarxiv (2022)). Briefly, the oligonucleotide pool was first amplified by limited-cycle PCR using Phusion Hot Start Flex 2× Master Mix, with an annealing temperature of 68° C. The T7 promoter sequence was introduced on the reverse primer during PCR. Further amplification was achieved by in-vitro transcription that was performed overnight using a high-yield in vitro transcription kit (NEB, cat. no. E2050S). Reverse transcription was then performed on the RNA template using Maxima H-Reverse Transcriptase (Thermo Fisher, cat. no. EP0753) to create a DNA-RNA hybrid. The RNA part was then cleaved off with alkaline hydrolysis, leaving behind a single-stranded DNA (ssDNA) which was then purified via magnetic bead purification and eluted in nuclease-free water (Ambion, cat. no. AM9930). The primers used for PCR are as follows:

### Mouse Kidney Library for FIG. 2:

Forward primer:
(SEQ ID NO: 53)
5′-CTATGCGCTATCCCGGACGC-3′
Reverse primer:
(SEQ ID NO: 54)
5′-TAATACGACTCACTATAGGGTCGCATATCCGTACCGGC-3′

### Mouse Cortex Library for FIG. 5:

Forward primer:
(SEQ ID NO: 55)
5′-CCGTTCAAGACTGCCGTGCTA-3′
Reverse Primer:
(SEQ ID NO: 56)
5′-TAATACGACTCACTATAGGGCTAGGGAGCCTACAGGCTGC-3′

### Mouse Cortex Library for FIG. 9:

Forward primer:
(SEQ ID NO: 57)
5′-TTGCGTTCGGTCTGAATGCG-3′
Reverse Primer:
(SEQ ID NO: 58)
5′-TAATACGACTCACTATAGGGACTCCTGCTCTTTGGGTCCG-3′

### Mouse Brain Library for FIG. 13:

Forward primer:
(SEQ ID NO: 59)
5′-CGCCCTAATCTCCGCTTGGG′-3′
Reverse Primer:
(SEQ ID NO: 60)
5′-TAATACGACTCACTATAGGGGCTTCGACCGAGGGCGAAAT′-3′

### Human Colorectal Cancer Library for FIG. 19:

Forward primer:
(SEQ ID NO: 61)
5′-TGCCCGCCTTTCGTTACTCA-3′
Reverse Primer:
(SEQ ID NO: 62)
5′-TAATACGACTCACTATAGGGCGCAATCGTCGGCTAACGGT-3′

### Coverslip Functionalization

Coverslip functionalization was performed as previously described in Goh, J. J. L. et al. (Goh, J. J. L. et al. Highly specific multiplexed RNA imaging in tissues with split-FISH. Nat Methods 17, 689-693 (2020)) and Lyubimova, A. et al. (Lyubimova, A. et al. Single-molecule mRNA detection and counting in mammalian tissue. Nat Protoc 8, 1743-58 (2013)). Briefly, coverslips (Warner Instruments, cat. no. 64-1500) were cleaned by gently shaking in 1 M KOH for 1 hour and rinsed thrice with MilliQ water. The coverslips were rinsed with 100% methanol, then immersed in an amino-silane solution (3% vol/vol (3-aminopropyl)triethoxysilane (Merck cat no. 440140), 5% vol/vol acetic acid (Sigma, cat. no. 537020) in methanol) for 2 minutes at room temperature before being rinsed three times with MilliQ water and dried in an oven at 47° C. overnight. Functionalized coverslips were then used immediately or stored in a dry, desiccated environment at room temperature for several weeks.

### Mouse Tissue Sample Preparation

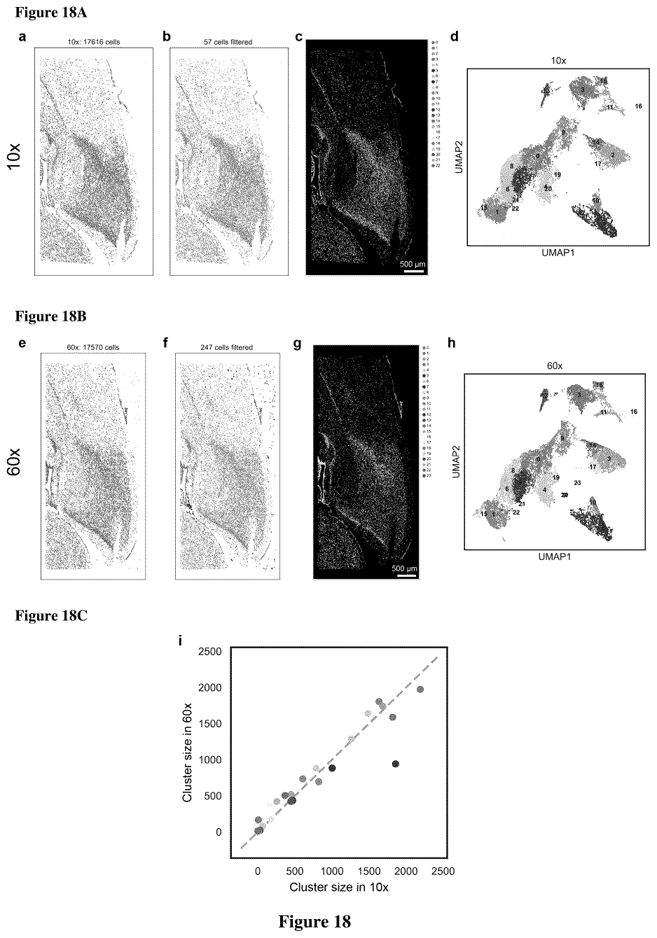

8-week-old C57BL/6nTAc female mice (In Vivos) were used in this study. All animal care and experiments were carried out in accordance with Agency for Science, Technology and Research (A*STAR) Institutional Animal Care and Use Committee (IACUC) guidelines (IACUC #211580). The mice were euthanized, and their kidneys and brains were quickly collected and frozen immediately in optimal cutting temperature compound (Tissue-Tek O.C.T.; VWR, cat. no. 25608-930), before storing at −80° C. The fresh frozen samples were then cut with a cryostat into 7 μm sections directly onto functionalized coverslips. For the comparison between 10× and 60× objectives (FIG. 18), adjacent mouse sagittal brain sections were used. Sections were air-dried for 5 minutes at room temperature before being fixed with 4% vol/vol paraformaldehyde in 1×PBS for 15 minutes. Following fixation, samples were rinsed once with 1×PBS and were either permeabilized immediately in 0.5% TritonX-100 in 1×PBS for 10 minutes at room temperature, or permeabilized in 70% ethanol overnight at 4° C., or stored at −80° C. No sample-size estimate was performed, since the goal was to demonstrate a technology.

### Human Colorectal Cancer Tissue Sample Preparation

As part of an ongoing research study approved by the institutional review boards of SingHealth (2020-186) for colorectal cancer (CRC), sample collection was carried out in accordance with ethical guidelines, and patients provided written, informed consent. To demonstrate the FISHnCHIPs technology, an aliquot from a non-individually identifiable tumor colon tissue was used (A*STAR IRB F-112), which was collected and frozen on dry ice immediately after resection and stored at −80° C. Prior to sectioning, tissue was embedded in optimal cutting temperature compound (Tissue-Tek O.C.T.; VWR, cat. no. 25608-930). Sections were obtained as described above, and following fixation, samples were rinsed once with 1×PBS before being permeabilized immediately in 70% ethanol overnight at 4° C. Sections were further permeabilized in 0.5% TritonX-100 in 1×PBS at room temperature for 15 minutes.

### Sample Staining

After permeabilization, the tissue sample was rinsed thrice with 1×PBS, followed by a rinse with 2×SSC. The encoding probes were diluted in a 20% or 30% hybridisation buffer to a final concentration of 1-2 nM per probe. The 20% hybridisation buffer composed of 20% deionized formamide (Ambion™ Cat: AM9342, AM9344) (vol/vol), 1 mg ml-1 yeast tRNA (Life Technologies, cat. no. 15401-011) and 10% dextran sulfate (Sigma, cat. no. D8906) (wt/vol) in 2×SSC. The sample was stained with the encoding probes for 16 to 48 hours at 37° C. or 47° C. Following hybridisation, the sample was washed in a 20% formamide wash buffer, containing 20% deionized formamide and 2×SSC, twice, incubating for 15-30 minutes at 37° C. or 47° C. per wash. The wash buffer was then removed, and the sample was washed twice with 2×SSC. The staining and washing conditions were optimized individually for each sample type. DAPI (Sigma, cat. no. D9564) was stained at a concentration of 1 μg/ml in 2×SSC for 10 minutes at room temperature. The sample was then washed thrice with 2×SSC and were either imaged immediately or stored at 4° C. in 2×SSC for no longer than 12 hours before imaging. For single-molecule FISH of DCN, MMP2, TAGLN, ACTA2, and SPARC (Biosearch technologies), the probes were diluted with 10% hybridisation buffer, and samples stained overnight at 37° C. Samples were than washed twice with a 10% formamide wash buffer for 15 minutes at 37° C. per wash, before rinsing with 2×SSC and subsequent imaging.

### Imaging Cycle

A flow chamber (Bioptechs, cat. no. FCS2) that could be secured to the microscope stage was used to mount the sample. Readout probe hybridisation was performed directly in the flow chamber by buffer exchange that was controlled by a custom-built, computer-controlled fluidics system as previously described in Chen, K. H., et al. (Chen, K. H., Boettiger, A. N., Moffitt, J. R., Wang, S. & Zhuang, X. Spatially resolved, highly multiplexed RNA profiling in single cells. Science 348, aaa6090 (2015)). All the buffer solutions (~1 ml per exchange) were flowed within 1 minute. 10 nM of fluorescently labelled readout probe in 10% high-salt hybridisation buffer was flowed into the chamber and incubated for 10 minutes at room temperature. The 10% high-salt hybridisation buffer composed of 10% deionized formamide (vol/vol) and 10% dextran sulfate (Sigma, cat. no. D8906) (wt/vol) in 4×SSC. Following hybridisation, the sample was rinsed with 2×SSC before flowing in 10% formamide wash buffer containing 0.1% TritonX-100. 2×SSC was flowed once more before imaging buffer. The imaging buffer consisted of 2×SSC, 10% glucose, 50 mM Tris-HCl pH 8, 2 mM Trolox (Sigma, cat. no. 238813), 0.5 mg/ml glucose oxidase (Sigma, cat. no. G2133) and 40 μg/ml catalase (Sigma, cat. no. C30). To remove the fluorescent signals, the samples were washed with 55% formamide wash buffer containing 0.1% TritonX-100. This hybridisation and wash cycle were repeated until all the readout probes were imaged.

### Imaging Set-Up 1

Imaging was performed on a step up described in Goh, J. J. L. et al. (supra). Briefly, the microscope was constructed around a Nikon Ti2-E body, Marzhauser SCANplus IM 130 mm×85 mm motorized X-Y stage, a Nikon CFI Plan Apo Lambda 60×1.4-n.a. oil-immersion objective, and an Andor Sona 4.2B-11 sCMOS camera. For the whole slide imaging experiment (FIG. 6), the Nikon CFI Plan Apo 10×0.5-n.a. water-immersion objective was used. The DAPI channel was excited by a Coherent Obis 405 100-mW laser. MPB Communications fiber lasers were used as illumination for Alexa594 (592 nm), Cy5 (647 nm) and IRDye 800CW (750 nm), respectively: 2RU-VFL-P-500-592-B1R (500 mW), 2RU-VFL-P-1000-647-B1R (1000 mW) and 2RU-VFL-P-500-750-B1R (500 mW). The Nikon Perfect Focus system was used to maintain focus while imaging, and in each imaging cycle, one Z position was imaged for each field of view. The Perfect Focus system was not used when imaging under the 10× water-immersion objective. Images were acquired at different exposure times (1 s, 500 ms, and 1 s with 60× and 3 s, 3 s, and 5 s with 10× for Alexa594, Cy5, and IRDye 800CW respectively) to avoid saturating the camera.

### Imaging Set-Up 2

A custom-built microscope constructed around a Nikon Ti2-E body, Marzhauser SCANplus IM 130 mm×85 mm motorized X-Y stage, and a pco.edge 4.2 BI-USB Back Illuminated sCMOS camera was used. A custom, fiber-coupled laser box from CNI laser was used as illumination for DAPI (405 nm), Alexa Fluor 488 (488 nm), Alexa Fluor 594 (588 nm), Cy5 (637 nm) and IRDye 800CW (750 nm). Custom multi-wavelength filters, 445/503/560/615/683/813 (Semrock) and 405/473/532/588/637/730 (Semrock), were used. The following objectives were tested: Nikon CFI Plan Apo Lambda 10×0.45-n.a. air objective (MRD00105), Nikon CFI Plan Apo 10×0.5-n.a. water-immersion objective (MRD71120), Nikon CFI Plan Fluor 20×0.75-n.a. water-immersion objective (MRH07241), Nikon CFI S Plan Fluor ELWD 20×0.45-n.a. air objective (MRH08230), Nikon CFI Apo LWD Lambda S 40×1.15-n.a. water-immersion objective (MRD77410), and Nikon CFI Plan Apo Lambda 60×1.4-n.a. oil-immersion objective (MRD01605). At 40× and 60×, the focus was maintained using the Nikon Perfect Focus system. One Z position was imaged per field of view. This set up is used for objective lenses comparison experiment and for immunofluorescence imaging.

### Immunofluorescence Staining

Tissues were rinsed with 1×PBS thrice at room temperature. Blocking was done with 1% BSA (NEB) and 0.1% Tween-20 in 1×PBS for 1 h at room temperature. Tissues were stained at 4° C. overnight using the following antibodies diluted in blocking solution: anti-LUM (Abcam, ab168384; 1:75), anti-MMP2 (Abcam, ab37150; 1:200), anti-α-SMA (Abcam, ab7817; 1:600), and anti-PDGFA (Santa Cruz Biotechnology, sc-9974; 1:600). PDPN was detected using AF488-conjugated primary antibody (BioLegend, 337005; 1:75). Secondary antibody staining was then carried out for 1 hour at room temperate using anti-mouse AF594 (ThermoFisher, A11005; 1:1000) and anti-rabbit AF488 (ThermoFisher, A11008; 1:1000). Finally, samples were stained with anti-CD68 (Cell Signalling Technology, #79594; 1:50) overnight at 4° C. After washing with 1×PBS three times, tissues were counterstained with DAPI (Sigma) before mounting (Vectashield, H-1700-10).

### Image Processing and Data Analysis

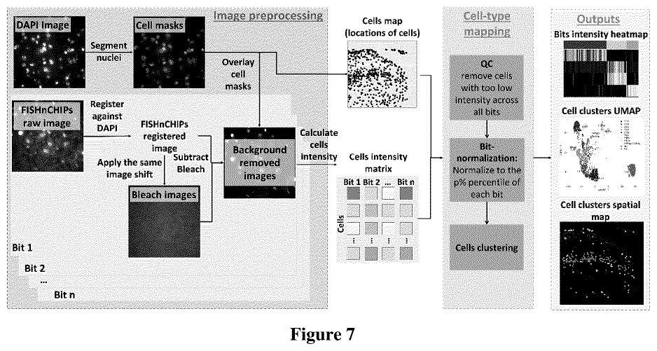

A custom pipeline (FIG. 7) was created to align the images (DAPI images, FISHnCHIPs images, and background images), segment, and cluster cell types. First, nuclei masks were obtained by performing nucleus segmentation using the deep learning based Cellpose algorithm (Stringer, C., Wang, T., Michaelos, M. & Pachitariu, M. Cellpose: a generalist algorithm for cellular segmentation. Nat Methods 18, 100-106 (2021)) or the watershed algorithm. The in situ hybridisation images were registered to the DAPI image by phase correlation using a subpixel registration algorithm provided in the Scikit-Image package (van der Walt, S. et al. scikit-image: image processing in Python. PeerJ 2, e453 (2014)). Subsequently, background images (after the 55% formamide wash, images were taken and used to estimate tissue autofluorescence background) were subtracted from the in situ hybridisation images after alignment (i.e., applying the same shifts). The nuclei masks obtained from the segmentation of DAPI were dilated to create cell masks, which were applied to all background subtracted in situ hybridisation images. An in situ hybridisation intensity matrix was constructed for cell type clustering and subsequent analyses. The intensity matrix was clustered using the Louvain algorithm after quality control and normalization. Cell clusters were visualized in a heatmap, dimensionality reduction plot, as well as a cluster map. The analysis pipeline is available for download as supplementary software.

### Gain and Crosstalk Analysis for Mouse Kidney

The nuclei segmentation and image alignment were performed as described above. Nuclei masks smaller than 3000 pixels were discarded. Nuclei masks were dilated by 5 pixels for creating cell masks. Images were normalized by dividing by the 99^{th} percentile of pixel intensities. A cell-by-channel-intensity matrix was constructed by calculating the mean fluorescence intensity per cell using the cell masks. Since only five kidney cell types were imaged in this experiment, cells with normalized intensity lower than 0.5 were dropped (keeping only ~18.6% of the cells that were brightly labelled by in situ hybridisation method described herein). Qualified cells with the highest normalized intensity across the channels were assigned to be the corresponding cell type. As shown in FIG. 2, the in situ hybridisation fluorescence signal gain was calculated by taking the ratio of the mean FISHnCHIPs intensity to the mean smFISH intensity in the same cell (the same cell masks were applied to both FISHnCHIPs and smFISH images as they were imaged sequentially on the same sample). The crosstalk of the in situ hybridisation method was estimated by calculating the Mander's overlap coefficient, a metric that quantifies the degree of co-localisation of objects in a pair of images (and was originally developed for dual-colour confocal microscopy). It is the fraction of overlap between two channels:

$${M}_{1} = \frac{Σ ( {C}_{1} > {t}_{1} ) \& ( {C}_{2} > {t}_{2} )}{Σ ( {C}_{1} > {t}_{1} )} ; {M}_{2} = \frac{Σ ( {C}_{1} > {t}_{1} ) \& ( {C}_{2} > {t}_{2} )}{Σ ( {C}_{2} > {t}_{1} )} ,$$

where t₁ and t₂ were the thresholds for binarizing the two channels C₁ and C₂ respectively.

### 18-Module Mouse Cortex Data Analysis

Gene-centric in situ hybridisation profiling of 18 gene modules in mouse cortex was conducted as shown in FIG. 5. The nuclei segmentation and image alignment were performed as described above. Nuclei masks smaller than 3000 pixels were discarded. Nuclei masks were dilated by 15 pixels for creating cell masks. Images were normalized to their 99^{th} percentile of pixel intensities. The cell-by-module-intensity matrix was constructed by taking the mean intensity of the segmented cell masks. Cells with total intensity lower than the 15^{th} percentile were removed for quality control. The cell-by-module-intensity matrix was used for clustering using the Seurat package. Modules were z-scaled before calculating principal components and dimensionality reduction projection. Clustering analysis was performed using the Louvain clustering algorithm. Cells were clustered at a resolution of 0.8 using the top 10 PCs with 20 nearest neighbours. Finally, the cell clusters were mapped back to the location of cell masks to reconstruct the spatial map.

### Mouse Cortex Neuronal Subtypes Data Analysis

The nuclei segmentation and image alignment were performed as described above. Nuclei masks smaller than 3000 pixels were discarded. Nuclei masks were dilated by 10 pixels for creating cell masks. Images were normalized to their 99^{th} percentile of pixel intensities. The cell-by-program-intensity matrix was constructed by taking the mean intensity of cell masks. Images were cropped to contain only the cortical region as shown in FIG. 9. Cells with total intensity lower than the 20th percentile were removed for quality control. The clustering analysis was performed as described above but at a higher resolution of 1.2. 5 out of 18 clusters (29.7% of the cells) contained cells with weak or no neuronal expression signature, which were then removed. As a result, 50.3% of all cells (defined by DAPI) were qualified as neurons. To quantify the cortical depth of neuron cells, edges from two circles with the same radius R=25,500 pixels were used to cover the regions with excitatory neurons as shown in FIG. 9. The distance between the two centres was 10,000 pixels. The normalized depth of cells was defined as the distance to the outer edge divided by the distance between the two centres. The cortical depth cell intensity heatmap was plotted by arranging cells with increasing depth (FIG. 11). The cell density along the cortical depth was estimated by applying a kernel density estimate (KDE) with a 0.05 Gaussian kernel.

### 53-Module Large FOV Mouse Brain Data Analysis

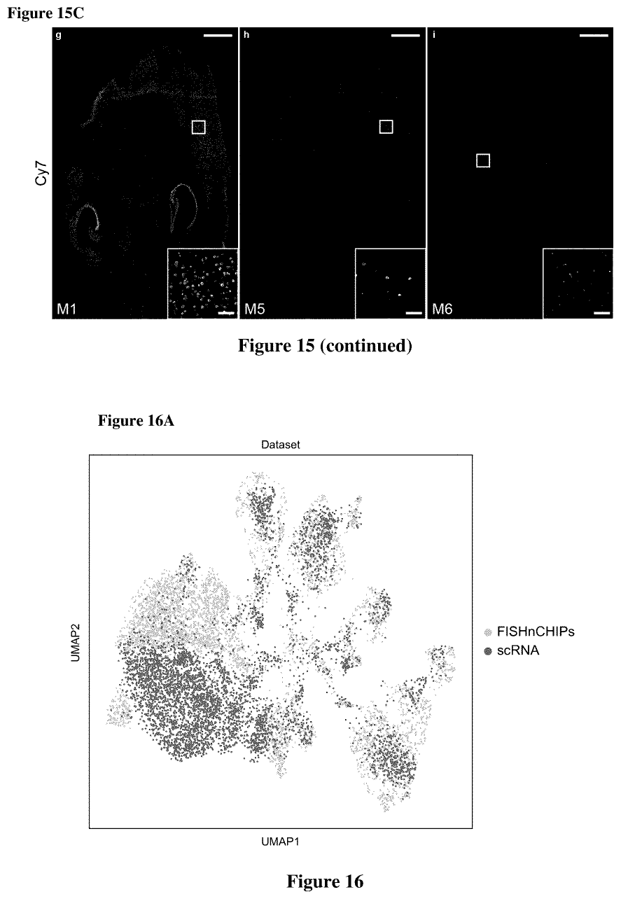

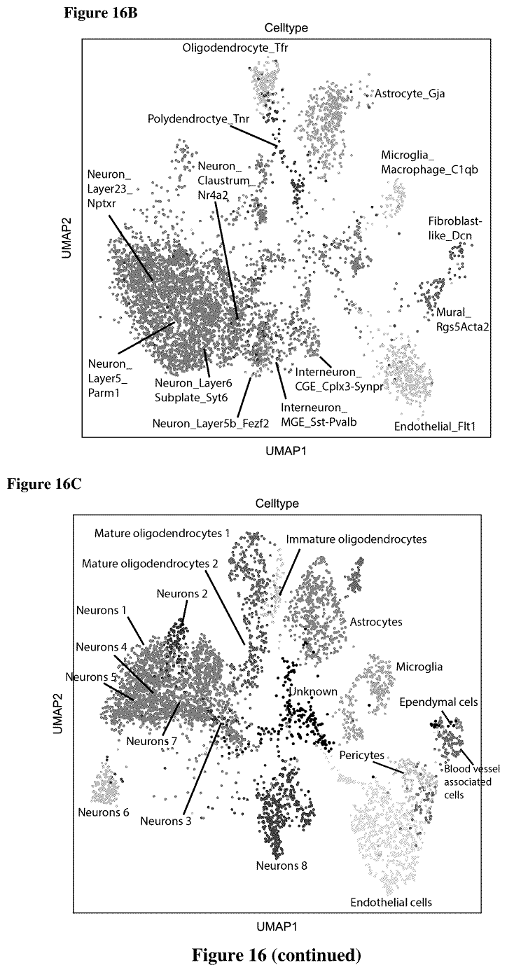

To generate the cell-by-module intensity matrix and cell positions of FIG. 13, the nuclei images were normalized to the 99^{th} percentile of pixel intensities and utilized the same nuclei segmentation pipeline as mentioned above. Each in situ hybridisation image was registered to their corresponding DAPI images, and the shifts were recorded. Shifts exceeding 50 pixels in any direction were discarded. The average shifts were then applied to all fields of view. To correct for illumination variations between fields of view, the 60^{th} percentile intensity of pixels outside the cell masks were subtracted. Cells with low intensity (<0.2%) across all modules, or with high intensity (>98%) across over 30 modules were removed. A graph of cells based on 15 nearest neighbours using the top 20 PCs were initially constructed. Leiden clustering performed at a resolution of 2. 133 cells (0.25%) from 2 of the preliminary clusters were affected by the autofluorescence of a dust particle in the sample and were dropped from further analysis. 54,834 (97.3%) qualified cells were clustered with a lower resolution of 0.6, resulting in 18 clusters or cell types. The blood vessel associated cells cluster and the inhibitory neurons cluster showed finer structure in the UMAP and were further sub-clustered. To verify the cluster annotations, integration analysis was performed using the Harmony algorithm (Korsunsky, I. et al. Fast, sensitive and accurate integration of single-cell data with Harmony. Nat Methods 16, 1289-1296 (2019)) between the in situ hybridisation method described and scRNA-seq (FIG. 16). To ensure compatibility, the in situ hybridisation data were cropped to the frontal cortex region. Additionally, the scRNA-seq data were subsampled randomly to balance the number of cells, following the recommendation by the Harmony authors. Normalization and scaling were applied to both scRNA-seq and in situ hybridisation data before integration. We were unable to annotate one of the clusters (2773 or 5% of the cells), as they exhibit low level expression across both the neuronal and non-neuronal modules and are spatially heterogeneous. From the integration analysis, these cells were observed to be in close proximity to the polydendrocytes and excitatory neuron clusters. Based on this observation, the ‘Unknown’ cluster is likely one or multiple genuine cell populations that was not resolved by the current probe set.

### Proximity of Cancer-Associated Fibroblasts (CAFs) to Immune Cells in Human Colorectal Cancer (CRC) Tissue

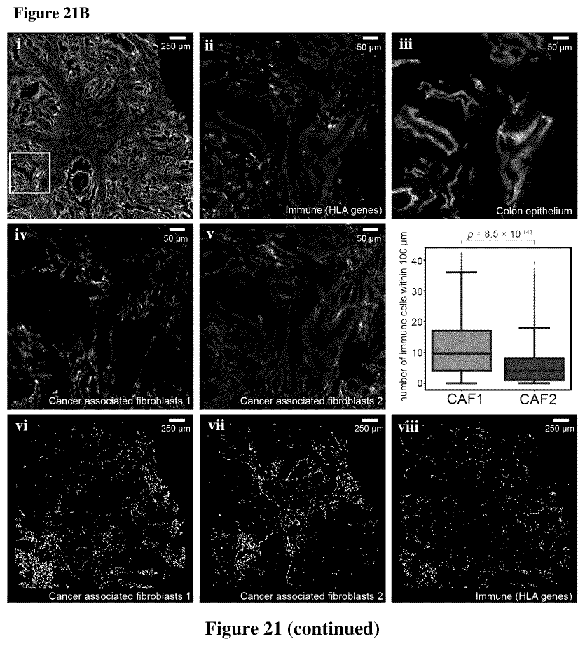

The fibroblasts and immune cells were segmented using the watershed segmentation algorithm provided in the Scikit-image package. The cut-off threshold and opening threshold for watershed segmentation were adjusted manually for each cell type. Using the centroids of the segmented cell masks, we calculated the number of immune cells within a 100 μm radius of CAF-1 or CAF-2 cells. As shown in FIG. 19, significantly greater numbers of immune cells were found closer to CAF-1 cells compared to CAF-2 cells (2-sided Mann-Whitney U test). This result was consistent with a visual inspection of cell positions (FIGS. 19 and 21).

### SUMMARY

In summary, the present disclosure demonstrated that the in situ hybridisation method as described herein can be used to robustly image and characterize cells within a biological tissue sample with high sensitivity and high throughput, while reducing the requirements and costs in experimental instruments.

1. A method of characterizing cells in a biological sample in situ, comprising:
a. contacting the biological sample with a plurality of probes that bind to ribonucleic acid (RNA) transcripts of a plurality of pre-determined genes, wherein each probe comprises
i) a detectable label, and
ii) a domain that binds specifically to a ribonucleic acid transcript of one of the pre-determined genes;
wherein a signal is emitted when the probe binds to the ribonucleic acid transcript;
b. detecting a combination or plurality of emitted signals from the plurality of probes; and
c. characterizing the cells based on the combination or plurality of emitted signals.

2. The method of claim 1, wherein steps a and b are repeated one or more times using a plurality of probes that bind to RNA transcripts of a plurality of different pre-determined genes.

3. The method according to claim 1, further comprising a step of quantifying the level of the emitted signal detected in step b, processing the signal, or both, prior to characterizing the cell.

4. The method according to claim 1, wherein the plurality of pre-determined genes comprises at least one gene and at least one other gene, wherein both show coordinated changes in their expression levels, where both are:
a) markers of a specific cell type;
b) differentially expressed genes of a specific cell type;
c) markers of a gene expression program or gene regulatory module;
d) markers of a biological pathway;
or combinations thereof; wherein the at least one other gene is selected from one or more input datasets.

5. The method according to claim 4, wherein the input dataset is a bulk RNA sequencing or single-cell RNA sequencing or microarray dataset or chromatin accessibility sequencing or methylation sequencing or DNA-associated proteins sequencing or spatial transcriptomics sequencing or multiplexed RNA fluorescence in situ hybridisation or multiplexed immunohistochemistry or bioinformatics database or any user-defined dataset or combinations thereof.

6. The method according to claim 1, wherein selection of the plurality of predetermined genes is an unsupervised selection, a supervised selection, or a combination thereof.

7. The method according to claim 4, wherein the coordinated changes in their expression levels of the at least one gene and at least one other gene is determined by correlation analysis or clustering analysis or dimensionality reduction analysis or differential expression gene analysis or combinations thereof of the input dataset.

8. The method according to claim 4, wherein the genes showing coordinated changes in their expression levels are further analyzed using signal gain (SG) or signal specificity ratio (SSR) to identify the plurality of pre-determined genes.

9. The method according to claim 1, wherein the domain of the probe is a ribonucleic acid (RNA) oligonucleotide that binds specifically to RNA.

10. The method according to claim 1, wherein the biological sample comprises a homogenous or heterogenous population of cells.

11. The method according to claim 1, wherein characterisation of the cell includes one or more of mapping the location of the cell in the biological sample, identifying an interaction between the cell and one or more other cells, identifying gene expression patterns of the cell or biological sample and visualizing the spatial transcriptome of the cell or biological sample, stratifying cancer subtypes, determine severity of cancer.

12. The method according to claim 1, further comprising pre-processing of the input dataset prior to performing correlation analysis or clustering analysis or dimensionality reduction analysis or differential expression gene analysis.

13. The method according to claim 1, wherein the plurality of pre-determined genes are expressed in cells associated with cancer.

14. A method to determine the prognosis of a subject suffering from cancer, comprising:
a. obtaining a sample of the subject;
b. characterizing one or more cancer cells in the sample using the method of claim 1 to determine the stage of the cancer; and
c. determining the prognosis based on the stage of the cancer.

15. A kit for characterising cells in a biological sample in situ comprising:
a plurality of probes that bind to ribonucleic acid (RNA) transcripts of a plurality of pre-determined genes; wherein each probe comprises
i) a detectable label, and
ii) a domain that binds specifically to a ribonucleic acid transcript of one of the pre-determined genes, and instructions for use.

16. The kit according to claim 15, wherein the plurality of probes bind to ribonucleic acid (RNA) transcripts of a plurality of pre-determined genes, wherein the plurality of pre-determined genes comprises at least one gene and at least one other gene that show coordinated changes in their expression levels, where both are:
e) markers of a specific cell type;
f) differentially expressed genes of a specific cell type;
g) markers of a gene expression program or gene regulatory module;
h) markers of a biological pathway;
or combinations thereof; wherein the at least one other gene is selected from one or more input datasets.

17. The kit according to claim 16, wherein the plurality of pre-determined genes are expressed in kidney, brain, cancer or combinations thereof.

18. (canceled)

19. The kit according to claim 15, wherein the cells are colorectal cancer cells.

20. The kit according to claim 15, wherein the plurality of pre-determined genes are selected from the genes listed in Table 6 (6a)-(6d).
